(19) United States
(12) Patent Application Publication
(10) Pub. No.: US 2023/0279290 A1
(43) Pub. Date: Sep. 7, 2023

(54) COMPOSITIONS AND METHODS FOR ASSAY MEASUREMENTS

(71) Applicant: Meso Scale Technologies, LLC., Rockville, MD (US)

(72) Inventors: John Kenten, Boyds, MD (US); John Pettersson, Rockville, MD (US); George Sigal, Rockville, MD (US); Alexander K. Tucker-Schwartz, Bethesda, MD (US)

(21) Appl. No.: 18/145,735

(22) Filed: Dec. 22, 2022

Related U.S. Application Data
(60) Provisional application No. 63/295,470, filed on Dec. 30, 2021.

(51) Int. Cl. C09K 11/02; C12Q 1/6816

(52) U.S. Cl. CPC C09K 11/02; C12Q 1/6816

(57) ABSTRACT

The disclosure relates to novel compositions comprising an electrochemiluminescence (ECL) co-reactant. In embodiments, the composition further comprises an ionic component, a surfactant, or combination thereof. In embodiments, the ECL co-reactant is triethanolamine (TEA), tert-butyldiethanolamine (tBDEA), methyldibutylethanolamine (MDEA), 3-[Bis-(2-hydroxy-ethyl)-amino]-propane-1-sulfonic acid (DEA-PS), or a combination thereof. Methods of using the compositions and kits comprising the compositions are also provided herein, including methods using ECL-labeled oligonucleotide probes having quenching moieties.

### CROSS-REFERENCE TO RELATED APPLICATIONS

This application claims the benefit of priority of U.S. Provisional Patent Application No. 63/295,470 filed Dec. 30, 2021, which is hereby expressly incorporated by reference in its entirety.

### REFERENCE TO SEQUENCE LISTING, TABLE, OR COMPUTER PROGRAM LISTING

The present application is being filed along with a Sequence Listing in electronic format. The Sequence Listing is provided as a file entitled MSD_002 A2_Sequence_Listing.xml, last saved Dec. 22, 2022, which is 8.61 kb in size. The information contained therein is incorporated herein by reference in its entirety.

### BACKGROUND

A number of commercially available instruments use electrochemiluminescence (ECL) for analytical measurements. Compounds that interact with the ECL label and generate ECL are referred to as ECL coreactants. Commonly used coreactants include tertiary amines (see, e.g., U.S. Pat. No. 5,846,485), oxalate, and persulfate for ECL from Ru(Bpy)₃⁺², and hydrogen peroxide for ECL from luminol (see, e.g., U.S. Pat. No. 5,240,863). The light generated by ECL labels can be used as a reporter signal in diagnostic procedures (see, e.g., U.S. Pat. No. 5,238,808). For instance, an ECL label can be covalently coupled to a detection reagent, and the participation of the detection reagent in a binding interaction can be monitored by measuring ECL emitted from the ECL label. Alternatively, the ECL signal from an ECL-active compound may be indicative of the chemical environment (see, e.g., U.S. Pat. No. 5,641,623 which describes ECL assays that monitor the formation or destruction of ECL coreactants). ECL-based assays are further described in U.S. Pat. Nos. 5,093,268; 5,147,806; 5,324,457; 5,591,581; 5,597,910; 5,641,623; 5,643,713; 5,679,519; 5,705,402; 5,846,485; 5,866,434; 5,786,141; 5,731,147; 6,066,448; 6,136,268; 5,776,672; 5,308,754; 5,240,863; 6,207,369; 5,589,136; and 6,919,173, and International Publication Nos. WO99/63347; WO00/03233; WO99/58962; WO99/32662; WO99/14599; WO98/12539; WO97/36931 and WO98/57154, each of which are herein incorporated by reference in its entirety.

Commercially available ECL instruments have become widely used because of their sensitivity, dynamic range, precision, and tolerance of complex sample matrices, among others. Several types of commercial instrumentation are available for performing ECL-based measurements (see, e.g., Debad, J. D., et al., 2004. Clinical and Biological Applications of ECL, in: Electrogenerated Chemiluminescence. Marcel Dekker, pp. 43-78). ECL instruments are further described, e.g., in U.S. Pat. Nos. 5,935,779 and 5,993,740 (bead-based ECL assays); U.S. Pat. Nos. 6,140,045; 6,066,448; 6,090,545; 6,207,369 and International Publication No. WO98/12539 (ECL assays using immobilized binding reagents); U.S. Pat. Nos. 6,977,722 and 7,842,246 (multi-well plates having integrated electrodes for ECL assays); and US Publication Nos. 2012/0190589 and US 2012/0178091 (cartridge-based ECL assays), each of which are herein incorporated by reference in its entirety.

The ECL coreactant tripropylamine (TPA) is typically used in ECL-based assays.

A molecular beacon is a molecular probe that is an oligonucleotide, which includes a stem and a loop, a quenching moiety, and a fluorescent moiety that interact in such a way that, in the absence of a target sequence, the quenching moiety reduces the fluorescent signal from the fluorescent moiety when the latter is approximate to the latter. Under ideal conditions, this molecular probe is able to generate a 200-fold increase in fluorescence on hybridization. However, molecular beacons of this type are typically limited to a single analyte (or in the above case, target sequence) that can be assayed at a given time. Furthermore, such molecular beacons with their fluorescent moieties are not easily controlled and have limited lifetimes due to photobleaching. Additionally, these types of molecular beacons require multiple cycles of incubation and washes, which may prolong the duration of the assay, and thus, lower efficiency. Examples of a molecular beacon probes having an ECL label are disclosed in Chem. Commun. 21:2710-2711 (2003) and Sensors & Actuators: B. Chemical 330:129261 (2021).

### SUMMARY

Embodiments of the disclosure include, an electrochemiluminescence (ECL) detection method comprising:
a) providing a substrate comprising an electrode and having a binding reagent immobilized on a surface of the substrate;
b) contacting the substrate with a composition, the composition comprising:
   i) a binding partner and/or binding complex comprising an oligonucleotide, wherein the binding reagent binds the binding partner and/or binding complex;
   ii) a plurality of ECL-labeled oligonucleotide probes comprising an oligonucleotide sequence that is complementary to an oligonucleotide sequence of the oligonucleotide of the binding partner and/or binding complex; and
   iii) an ECL co-reactant that is not TPA;
c) allowing a portion of the plurality of ECL-labeled oligonucleotide probes to hybridize to the oligonucleotide of the binding partner and/or binding complex, wherein the binding partner and/or binding complex is bound by the binding reagent, and wherein another portion of the plurality of ECL-labeled oligonucleotide probes is not hybridized to the oligonucleotide of the binding partner and/or binding complex bound by the binding reagent;
d) selectively dequenching the portion of the plurality of ECL-labeled probes hybridized to the oligonucleotide of the binding partner and/or binding complex;
e) applying a voltage to the electrode to generate ECL; and
f) measuring the ECL wherein the portion of the plurality of ECL-labeled oligonucleotide probes that is not hybridized to the oligonucleotide of the binding partner and/or binding complex is not removed from the composition prior to applying the voltage and measuring the ECL.

In embodiments, b) contacting the substrate with the composition comprises:
b′) contacting the substrate with a composition comprising the binding partner and/or binding complex;
b″) contacting the substrate with a composition comprising the plurality of ECL-labeled oligonucleotide probes; and
b′″) contacting the substrate with a composition comprising the ECL co-reactant.

In embodiments, each of steps b′), b″) and b′″) are carried out sequentially. In embodiments, at least two of steps b′), b″) and b′″) are carried out simultaneously. In embodiments, the method comprises:
b′) contacting the substrate with a first composition comprising the binding partner and/or binding complex, and allowing the binding partner and/or binding complex to immobilize on the surface by binding to the binding reagent; and
b″) contacting the substrate comprising the immobilized binding partner and/or binding complex with a second composition comprising the plurality of ECL-labeled oligonucleotide probes and the ECL co-reactant; or
b′) contacting the substrate with a first composition comprising the binding partner and/or binding complex and the plurality of ECL-labeled oligonucleotide probes, wherein a portion of the plurality of ECL-labeled oligonucleotide probes are hybridized to the oligonucleotide of the binding partner and/or binding complex, and allowing the binding partner and/or binding complex to immobilize on the surface by binding to the binding reagent; and
b″) contacting the substrate comprising the immobilized binding partner and/or binding complex with a second composition comprising the ECL co-reactant; or
b′) contacting the substrate with a first composition comprising the binding partner and/or binding complex and allowing the binding partner and/or binding complex to immobilize on the surface by binding to the binding reagent; and
b″) contacting the substrate comprising the immobilized binding partner and/or binding complex with a second composition comprising the plurality of ECL-labeled oligonucleotide probes and allowing a portion of the plurality of ECL-labeled oligonucleotide probes to hybridize to the oligonucleotide of the immobilized binding partner and/or binding complex; and
b″) contacting the substrate with a third composition comprising the ECL co-reactant.

In embodiments, further comprising washing the substrate following the contacting the substrate with the binding partner and/or binding complex to remove binding partner and/or binding complex not bound by the binding reagent, wherein the washing is prior to contacting the substrate with the composition comprising the plurality of ECL-labeled oligonucleotide probes.

Embodiments of the disclosure include an electrochemiluminescence (ECL) detection method comprising:
a) providing a substrate comprising an electrode and having a binding partner and/or binding complex comprising an oligonucleotide immobilized on a surface of the substrate;
b) contacting the substrate with a composition, the composition comprising:
   i) a plurality of ECL-labeled oligonucleotide probes comprising an oligonucleotide sequence that is complementary to an oligonucleotide sequence of the oligonucleotide of the binding partner and/or binding complex; and
   ii) an ECL co-reactant that is not TPA;
c) allowing a portion of the plurality of ECL-labeled oligonucleotide probes to hybridize to the oligonucleotide of the immobilized binding partner and/or binding complex, and wherein another portion of the plurality of ECL-labeled oligonucleotide probes is not hybridized to the oligonucleotide of the immobilized binding partner and/or binding complex;
d) selectively dequenching the portion of the plurality of ECL-labeled probes hybridized to the oligonucleotide of the binding partner and/or binding complex;
e) applying a voltage to the electrode to generate ECL; and
   f) measuring the ECL wherein the portion of the plurality of ECL-labeled oligonucleotide probes that is not hybridized to the oligonucleotide of the binding partner and/or binding complex is not removed from the composition prior to applying the voltage and measuring the ECL.

In embodiments, b) contacting the substrate with the composition comprises:
b′) contacting the substrate with a composition comprising the plurality of ECL-labeled oligonucleotide probes; and
b″) contacting the substrate with a composition comprising the ECL co-reactant.

In embodiments, each of steps b′) and b″) are carried out sequentially. In embodiments, each of steps b′) and b″) are carried out simultaneously, optionally wherein the plurality of ECL-labeled oligonucleotide probes and the ECL co-reactant are in a single composition.

Embodiments of the disclosure, including those in the preceding paragraphs, include embodiments wherein the binding partner and/or binding complex comprises an analyte. In embodiments, the analyte comprises a peptide. In embodiments, the analyte comprises an oligonucleotide. In embodiments, the analyte is the oligonucleotide of the binding partner and/or binding complex. In embodiments, the analyte is labeled with the oligonucleotide. In embodiments, the analyte is labeled with the oligonucleotide by binding the analyte with a detection reagent comprising the oligonucleotide. In embodiments, the oligonucleotide of the binding partner and/or binding complex comprises multiple copies of the sequence complementary to the oligonucleotide sequence of the plurality of the ECL-labeled oligonucleotide probes. In embodiments, prior to contacting the substrate with the plurality of the ECL-labeled oligonucleotide probes, performing an amplification reaction to generate the multiple copies of the sequence complementary to the oligonucleotide sequence of the plurality of the ECL-labeled oligonucleotide probes. In embodiments, the analyte is labeled with the oligonucleotide by binding the analyte with a detection reagent comprising an oligonucleotide primer, and wherein the oligonucleotide primer is extended by a polymerase to generate the oligonucleotide that comprises the multiple copies of the sequence complementary to the oligonucleotide sequence of the ECL-labeled oligonucleotide probes. In embodiments, the amplification reaction or primer extension is a rolling circle amplification reaction. In embodiments, the ECL-labeled oligonucleotide probes include a stem-loop or hairpin structure, an ECL label, and a quenching moiety, wherein the quenching moiety is in proximity to the ECL label and quenches the ECL label when the oligonucleotide probe is in a stem-loop or hairpin configuration, but does not quench the ECL label when the stem-loop or hairpin structure is in an open configuration, and wherein the selectively dequenching comprises hybridizing the portion of the plurality of ECL-labeled oligonucleotide probes to the to the oligonucleotide of the binding partner and/or binding complex in the open configuration. In embodiments, the ECL-labeled oligonucleotide probes comprise an ECL label and a quenching moiety, wherein the quenching moiety is in proximity to the ECL label and quenches the ECL label when the oligonucleotide probe is in a linear confirmation, wherein the selectively dequenching comprises selectively cleaving the quenching moiety from only the portion of the plurality of ECL-labeled probes hybridized to the oligonucleotide of the binding partner and/or binding complex such that the quenching moiety is released into solution and is no longer in proximity to the ECL label of the hybridized ECL-labeled probe which remains hybridized to the oligonucleotide of the binding partner and/or binding complex after cleavage of the quenching moiety. In embodiments, the cleaving is performed by an enzyme. In embodiments, the enzyme is selected from the group consisting of a nicking restriction endonuclease, an RNaseH2, and a polymerase having 5′ exonuclease activity. In embodiments, the enzyme cleaves only the ECL-labeled oligonucleotide probe leaving the oligonucleotide of the binding partner and/or binding complex intact. In embodiments, the enzyme is a nicking restriction endonuclease that recognizes a sequence in the hybridized ECL-labeled probe, or an RNaseH2 which recognizes an RNA base in the hybridized ECL-labeled probe. In embodiments, the enzyme is a polymerase having 5′ exonuclease activity, and wherein the method further comprises: hybridizing a primer to the oligonucleotide of the binding partner and/or binding complex at a position 5′ of the hybridized ECL-labeled probe, allowing the polymerase having 5′ exonuclease activity to extend the primer to the hybridized ECL-labeled probe, wherein the 5′ exonuclease activity cleaves the quenching moiety of the hybridized ECL-labeled probe, and wherein the ECL-labeled probe comprises a portion that is resistant to the 5′ exonuclease activity. In embodiments, the ECL co-reactant is selected from the group consisting of 3-(di-n-propylamino)-propanesulfonic acid; 4-(di-n-propylamino)-butanesulfonic acid; 4-[bis-(2-hydroxyethane)-amino]-butanesulfonic acid; piperidine-N-(3-propanesulfonic acid); azepane-N-(3-propanesulfonic acid); piperidine-N-(3-propionic acid) (PPA); 3-morpholino-2-hydroxypropanesulfonic acid (MOPSO); 3-morpholinepropanesulfonic acid (MOPS); N-(2-hydroxyethyl)piperazine-N′-3-propanesulfonic acid (EPPS); N-(2-hydroxyethyl)piperazine-N′-3-ethanesulfonic acid (BES); piperazine-N,N′-bis(2-ethanesulfonic acid) (PIPES); triethanolamine (TEA); N-2-hydroxypiperazine-N-2-ethanesulfonic acid (HEPES); piperazine-N,N′-bis-4-butanesulfonic acid; homopiperidine-N-3-propanesulfonic acid; piperazine-N,N′-bis-3-propanesulfonic acid; piperidine-N-3-propanesulfonic acid; piperazine-N-2-hydroxyethane-N′-3-methylpropanoate; piperazine-N,N′-bis-3-methylpropanoate; 1,6-diaminohexane-N,N,N′,N′-tetraacetic acid; N,N-bis propyl-N-4-aminobutanesulfonic acid; N-tris(hydroxymethyl)methyl-2-aminoethane sulfonic acid (TES); 1,3-bis[tris(hydroxymethyl)methylamino]propane (bis-Tris propane); 3-dimethylamino-1-propanol; 3-dimethylamino-2-propanol; N,N,N′,N′-tetrapropylpropane-1,3-diamine (TPA dimer); piperazine-N,N′-bis(2-hydroxypropane)sulfonic acid (POPSO) and 2-hydroxy-3-[4-(2-hydroxyethyl)piperazin-1-yl]propane-1-sulfonic acid (HEPPSO), N-butyldiethanolamine (BDEA) 2-dibutylaminoethanol (DBAE), tert-butyldiethanolamine (tBDEA), methyldiethanolamine (MDEA), 3-[Bis-(2-hydroxy-ethyl)-amino]-propane-1-sulfonic acid (DEA-PS), and combinations thereof. In embodiments, the ECL co-reactant is TEA, or is selected from: the group consisting of TEA, tBDEA, BDEA, MDEA, DEA-PS, and combinations thereof; the group consisting of TEA, tBDEA, MDEA, DEA-PS, and combinations thereof; or the the group consisting of TEA, tBDEA, MDEA, DEA-PS, and combinations thereof. In embodiments, the quenching moiety is selected from the group consisting of ATTO 540Q, ATTO 575Q, ATTO 580Q, ATTO 612Q, Iowa Black FQ, Iowa Back RQ, QSY 21, IRDye QC-1, BHQ0, BHQ1, BHQ-2, BHQ-3, Dabcyl, QSY 7, QSY 9, QSY 21, QSY 35, QXL 490, QXL 520, QXL 570, QXL 670, ferrocene, iron bipyridine, and combinations thereof. In embodiments, the ECL label is an organometallic complex comprising ruthenium, osmium, iridium, rhenium, and/or lanthanide metal. the ECL label comprises tris(bipyridine)ruthenium or a modified tris(bipyridine)ruthenium. the ECL label comprises the chemical structure shown in Formula II:

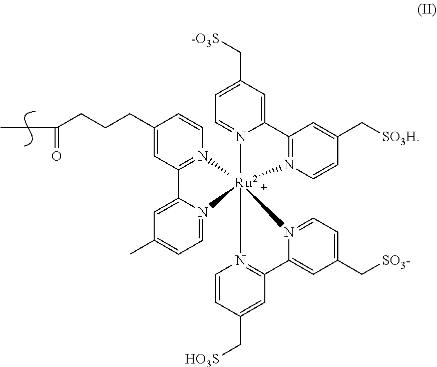

In embodiments, the co-reactant is in a composition of any one of the embodiments above and herein. In embodiments, the co-reactant is in a composition of any one of numbered items 1-69. In embodiments, the co-reactant comprises TEA.

### BRIEF DESCRIPTION OF THE DRAWINGS

The following drawings form part of the present specification and are included to further demonstrate exemplary embodiments of certain aspects of the present disclosure.

FIGS. 1A and 1B relate to Example 1 and show the results of an embodiment of an ECL-based assay. A panel of ECL coreactants combined with one of two surfactants was tested for ECL generation and ability to discriminate between surface-bound (“BTI”) and free (in solution; “FT”) ECL labels in a solid-surface ECL assay. FIG. 1A shows the ECL signal measured with BTI, FT, and background signal (“D100”) with ECL read buffer only (no label). FIG. 1B shows the ratio of ECL signal from bound label to ECL signal from free label (“BTI/FT”), and the signal-to-background ratio (“S/B”).

FIGS. 2A-2C relate to Example 2 and show the results of an embodiment of an ECL-based assay. FIG. 2A shows a plot of the ECL generated from BTI and FT, and the BTI/FT ratio with varying concentration of TEA. FIG. 2B shows the measured ECL signals from BTI, FT, and background (D100) with varying concentrations of TEA. FIG. 2C shows the BTI/FT ratio, S/B ratio, and percent ECL generation compared with PIPES ECL read buffer.

FIGS. 3A and 3B relate to Example 3 and show the results of an embodiment of an ECL-based assay. FIG. 3A shows the change in ECL signal as a function of PIPES concentration.

FIG. 3B shows the change in ECL signal as a function of PIPES or TEA concentration.

FIGS. 4A-4H illustrate embodiments of ECL-based assays described herein and are further described in Example 4.

FIG. 4A illustrates a “standard” 2-step washed assay, wherein a capture antibody (“cAb”; binding reagent) immobilized on a surface is contacted with a mixture of analytes, one of which binds specifically to the capture antibody, and the surface is then washed, resulting in the analyte captured on the surface. A mixture of detection antibodies (“dAb”; detection reagent), each containing an ECL label and one of which binds specifically to the analyte, is then added to the surface, and the surface is then washed, resulting in a binding complex comprising the cAb, analyte, and dAb. ECL read buffer is then added to the surface, and the generated ECL is then read by an ECL reader instrument.

FIG. 4B illustrates a “1-step” assay, wherein a capture antibody on a surface is contacted with an analyte mix, and the surface is then washed as in FIG. 4A. The detection antibody mix is then added, followed by the ECL read buffer without washing in between adding the detection antibody mix and the ECL read buffer. The generated ECL is then read by an ECL reader instrument.

FIG. 4C illustrates a “1-step non-wash” assay, wherein a capture antibody on a surface is contacted with: an analyte mix and detection antibody mix, followed by the ECL read buffer without washing in between any of the steps. The generated ECL is then read by an ECL reader instrument.

FIG. 4D illustrates a “mock ECL label” assay, wherein a capture antibody on a surface is contacted with an analyte mix, the surface is washed, and a detection antibody mix is added, the surface is optionally washed again, resulting in a binding complex as in FIG. 4A. The ECL read buffer is then added to the surface along with a detection antibody that comprises an ECL label and that does not bind to any component of the binding complex on the surface, which serves as a proxy for “free” ECL label in solution. The generated ECL is then read by an ECL reader instrument.

FIG. 4E illustrates a multiplexed version of the “standard” 2-step washed assay, wherein one or more surfaces comprises a plurality of binding domains, each binding domain comprising a capture antibody that can bind to an analyte in the analyte mix. The surface(s) comprising the binding domains is washed after adding the analyte mix, resulting in a plurality of analytes captured on the binding domains. A mixture of detection antibodies, each containing an ECL label and capable of binding to an analyte in the analyte mix, is then added to the surface(s), and the surface is then washed, resulting in a plurality of binding complexes, each binding complex comprising a cAb, analyte, and dAb. ECL read buffer is then added to the surface, and the generated ECL is read by an ECL instrument.

FIG. 4F illustrates a multiplexed version of the “1-step” assay, wherein one or more surfaces comprises a plurality of binding domains, each binding domain comprising a capture antibody that can bind to an analyte in the analyte mix. The surface(s) comprising the binding domains is washed after adding the analyte mix as in FIG. 4E. The detection antibody mix is added to form a plurality of binding complexes, and ECL read buffer is then added without washing in between adding the detection antibody mix and the ECL read buffer. The generated ECL is then read by an ECL reader instrument.

FIG. 4G illustrates a multiplexed version of the “1-step non-wash” assay, wherein one or more surfaces comprises a plurality of binding domains, each binding domain comprising a capture antibody that can bind to an analyte in the analyte mix. The surface(s) comprising the binding domains is contacted with an analyte mix and detection antibody mix to form a plurality of binding complexes, then ECL read buffer is added without washing in between any of the steps. The generated ECL is then read by an ECL reader instrument.

FIG. 4H illustrates a multiplexed version of the “mock ECL label” assay, wherein one or more surfaces comprises a plurality of binding domains, each binding domain comprising a capture antibody that can bind to an analyte in the analyte mix. The surface(s) comprising the binding reagents is contacted with an analyte mix, the surface is washed, a detection antibody mix is added, and the surface is optionally washed again, resulting in a plurality of binding complexes as in FIG. 4E. The ECL read buffer is then added to the surface along with a detection antibody that comprises an ECL label and that does not bind to any component of the binding complex on the surface, which serves as a proxy for “free” ECL label in solution. The generated ECL is then read by an ECL reader instrument.

FIGS. 5A-5D relate to Example 5A and show the results of an embodiment of an ECL-based assay. FIG. 5A shows the results of specific ECL signal and non-specific binding (NSB) from three different multiplexed assay formats (shown in FIGS. 4E, 4F, and 4H) with BDEA, PIPES, and TEA read buffers. FIG. 5B shows the lowest limit of detection (LLOD) of the assays in FIG. 5A. FIG. 5C shows a relative comparison of the ECL and NSB results from FIG. 5A. FIG. 5D shows the comparison of signal to background (S/B) and signal to noise (S/N) ratio across all ECL read buffers and assay formats.

FIGS. 6A-6C relate to Example 5B and show the results of an embodiment of an ECL-based assay. FIG. 6A shows the results of specific ECL signal and non-specific binding (NSB) from three different multiplexed assay formats (shown in FIGS. 4E, 4F, and 4G) with BDEA, PIPES, and TEA read buffers. FIG. 6B shows the lowest limit of detection (LLOD) of the assays in FIG. 6A. FIG. 6C shows a relative comparison of the ECL and NSB results from FIG. 6A.

FIGS. 7A-11B relate to Example 6 and show the results of an embodiment of an ECL-based assay.

FIG. 7A shows a list of sample matrices tested with TEA read buffer in a 1-step non-wash ECL assay. FIG. 7B shows a list of interferents added to the sample matrices in FIG. 7A, to be tested with TEA read buffer in a 1-step non-wash ECL assay.

FIG. 8A shows the results of ECL signal generated from TEA read buffer with bound ECL label (“Bound”) and free ECL label (“Free”), with different sample matrices mixed with diluent. “H2O” indicates signal from a control with water instead of a sample matrix prior to TEA read buffer. The column headers with “Free” indicates 6 nM of free ECL label in diluent.

FIG. 8B shows the results of FIG. 8A normalized to ECL signal generated from an assay in which sample matrices were not added.

FIG. 9A shows the results of ECL signal generated from TEA read buffer with bound and free ECL label with different interferents in different sample matrices. FIG. 9B shows the results of FIG. 9A normalized to ECL signal generated from an assay in which sample matrices and interferents were not added.

FIG. 10A shows the results of ECL signal generated from TEA read buffer with free ECL label (“D3+STAG”) in different sample matrices. The column headers with “Free” indicates 240 nM of free ECL label in diluent. FIG. 10B shows the results of FIG. 10A normalized to ECL signal generated from an assay in which sample matrices were not added.

FIG. 11A shows results of ECL signal generated from TEA read buffer with 240 nM of free ECL with different interferents in different sample matrices. FIG. 11B shows the results of FIG. 11A normalized to ECL signal generated from an assay in which sample matrices and interferents were not added.

FIG. 12 relates to Example 7 and shows the results of an embodiment of an ECL-based assay. Combinations of ECL coreactants described in Example 1 were tested in an ECL-based assay. The top-right side of the chart in FIG. 12 shows the ECL signal generated from BTI, while the bottom-left side of the chart in FIG. 12 shows the ECL signal ratio of the mixed ECL coreactants to the sum of signal generated by the individual ECL coreactants.

FIGS. 13A and 13B relate to Example 8 and show the results of an embodiment of an ECL-based assay. The ECL coreactants described in Example 1 were tested for sensitivity to the presence of TRITON™ X-100. FIG. 13A shows the ECL signal from BTI and FT for each ECL reactant in TRITON™ X-100 (TX100) and PEG(18) tridecyl ether (PEG18TDE). FIG. 13B shows the ratio of ECL generated in TRITON™ X-100 vs. PEG(18) tridecyl ether.

FIG. 14 is an illustration of an embodiment of a method for detecting target oligonucleotide analyte(s) utilizing an ECL-labeled molecular beacon oligonucleotide probe and a co-reactant (co-reactant not shown).

FIG. 15 is an illustration of an embodiment of a method for detecting target oligonucleotide analyte(s) utilizing an ECL-labeled molecular beacon oligonucleotide probe and a TEA co-reactant (co-reactant not shown) as described in Examples 10-12.

FIG. 16 is an illustration of embodiments of ECL-labeled molecular beacon probes prepared in Example 9 and used in the experiments described in Examples 10-12.

FIGS. 17A-17D are graphical representations of the results of an embodiment of a wash-free experiment described in Example 10 using the ECL-labeled molecular beacon probes of Example 9. FIG. 17A depicts the ECL signals in the presence of increasing concentration of target oligonucleotide that is free in solution using a read buffer containing TPA. FIG. 17B depicts the ECL signals in the presence of increasing concentration of target oligonucleotide that is immobilized on the surface of a substrate using a read buffer containing TPA. FIG. 17C depicts the ECL signals in the presence of increasing concentration of target oligonucleotide that is free in solution using a read buffer containing TEA. FIG. 17D depicts the ECL signals in the presence of increasing concentration of target oligonucleotide that is immobilized on the surface of a substrate using a read buffer containing TEA.

FIGS. 18A-18B report the results of an embodiment a wash-free broad concentration range experiment described in Example 11 using the ECL-labeled molecular beacon probes of Example 9. FIG. 18A depicts the ECL signals in the presence of increasing concentration of target oligonucleotide that is immobilized on the surface of a substrate using a read buffer containing TEA. FIG. 18B is a chart listing the ECL signal intensities depicted in FIG. 18A for each MB probe with decreasing concentrations of target oligonucleotide.

FIGS. 19A-19B report the results of an embodiment of a 2-step wash-free experiment described in Example 12 using the ECL-labeled molecular beacon probes of Example 9. FIG. 19A depicts the ECL signals in the presence of increasing concentration of target oligonucleotide that is immobilized on the surface of a substrate using a read buffer containing TEA. FIG. 19B is a chart listing the ECL signal intensities depicted in FIG. 18A for each MB probe with decreasing concentrations of target oligonucleotide.

FIG. 20 is an illustration of an embodiment of a method for detecting a peptide analyte indirectly labeled with an oligonucleotide primer, wherein the method utilizes an ECL-labeled molecular beacon oligonucleotide probe that binds to an extended sequence originating from the primer. No wash step is performed after the addition of the ECL-labeled oligonucleotide probe. In this embodiment, streptavidin is a binding reagent, and the biotinylated capture antibody, peptide analyte, and second antibody comprising an extended oligonucleotide primer, together form a binding complex.

FIG. 21 is an illustration of an embodiment of a method for detecting target oligonucleotide analyte(s) utilizing an ECL-labeled oligonucleotide probe where the quenching moiety is selectively cleaved from the hybridized probe by an enzyme, e.g., a nicking endonuclease, dequenching the ECL label of the probe while leaving the remainder of the ECL-labeled probe hybridized to the target oligonucleotide.

### DETAILED DESCRIPTION DISCLOSURE

ECL coreactants of the present disclosure provide consistent ECL generation across different assay formats. It was discovered that the compositions herein, e.g., comprising triethanolamine (TEA), are useful in ECL-based assays that do not require a wash step. Many ECL-based assays conducted on solid surfaces involve at least one wash step to remove unbound ECL labels prior to detecting the ECL labels on the surface (i.e., a “washed” assay). The wash step may be eliminated if the detection method can effectively discriminate between an ECL label bound to the surface (e.g., as part of a binding complex to be detected) or an unbound, “free” ECL label in solution. A “non-wash” assay format, which eliminates the wash step, is often advantageous because the washing step can be difficult or cumbersome to perform in many circumstances. However, a non-wash assay format is typically difficult to develop due to high background ECL signal from incomplete discrimination of free vs. bound ECL labels present in the reaction mixture.

In ECL-based assays conducted on solid surfaces, triethanolamine (TEA) was surprisingly discovered to discriminate between unbound (“free”) ECL labels in solution, versus surface-bound ECL labels to high degree. Compositions described herein, comprising TEA, increase the ratio of ECL signal from bound label to ECL signal from free label relative to conventional to compositions comprising conventional coreactants such as tripropylamine (TPA). Thus, the compositions herein provide improved assay performance, particularly when measuring low affinity interactions, which require the presence of the ECL label in high concentrations in the reaction, but would also be expected to suffer from significant signal loss due to binding complex dissociation during wash steps.

ECL signal generated from the compositions herein, e.g., comprising TEA, tert-butyldiethanolamine (tBDEA), methyldiethanolamine (MDEA), and/or 3-[Bis-(2-hydroxy-ethyl)-amino]-propane-1-sulfonic acid (DEA-PS) provide further advantages such as improved consistency in performance between compositions that differ based on the presence or absence or surfactant, or based on the surfactant identity. In particular, the compositions perform similarly when containing no surfactant, when containing a mild surfactant that does not disrupt lipid bilayer membranes (such as polyethylene glycol (18) tridecyl ether), or when containing a harsher surfactant (such as TRITON™ X-100). Thus, a harsh surfactant (e.g., TRITON™ X-100, which disrupts lipid bilayer membranes that part of certain analytes of interest such as whole cells or extracellular vesicles) is not required in the compositions comprising the ECL coreactants described herein, which is in contrast to tripropylamine (TPA), a typical ECL coreactant that usually requires TRITON™ X-100 for optimal ECL generation. Thus, the compositions herein are useful in assays to detect analytes that are sensitive to harsh surfactants. Moreover, the ECL coreactants ECL signals are not greatly affected by the presence of different surfactants, and thus, these ECL coreactants are versatile and can be easily incorporated in different formulations while maintaining their ECL generation capabilities.

Thus, the compositions herein, e.g., comprising TEA, tBDEA, MDEA, and/or DEA-PS advantageously expand the types of ECL-based assays that can be performed.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers (including fractional numbers and whole numbers) that fall within x and y. Moreover, reference herein to a range of from “5 to 10” includes whole numbers of 5, 6, 7, 8, 9, and 10, and fractional numbers 5.1, 5.2, 5.3, 5.4, 5.5, 5.6, 5.7, 5.8, 5.9, etc. Reference to any numerical range expressly includes each numerical value (including fractional numbers and whole numbers) encompassed by that range. To illustrate, a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

As used herein, the term “substantially,” or “substantial,” is applicable when used in a negative connotation to refer to the complete or near complete lack of an action, characteristic, property, state, structure, item, or result. For example, a surface that is “substantially” flat would be either completely flat, or so nearly flat that the effect would be the same as if it were completely flat. In a further example, a composition that is “substantially” free of a certain component would not have any amount of that component, or the component would be present in such a low amount in the composition that the effect would be the same as if the component was not present.

In embodiments, the disclosure provides a composition comprising: (a) triethanolamine (TEA); and (b) an ionic component; wherein the composition has a pH of about 7.0 to about 8.0, and wherein the composition is substantially free of an additional pH buffering component.

In embodiments, the disclosure provides a composition comprising: (a) triethanolamine (TEA); (b) an ionic component; and (c) an ECL-labeled component; wherein the composition has a pH of about 7.0 to about 8.0, and wherein the composition is substantially free of an additional pH buffering component.

In embodiments, the disclosure provides a composition comprising: (a) about 1000 mM to about 6500 mM of triethanolamine (TEA); and (b) about 500 mM to about 2000 mM of an ionic component; wherein the composition has a pH of about 7.0 to about 8.0.

In embodiments, the disclosure provides a composition comprising: (a) triethanolamine (TEA); (b) an ionic component; (c) an alkyl ether-polyethylene glycol (PEG); wherein the composition has a pH of about 7.0 to about 8.0.

In embodiments, the disclosure provides a composition comprising (a) TEA, (b) an ionic component; and (c) optionally, one or both of an ECL-labeled component and a surfactant, wherein the composition has a pH of about 7.0 to about 8.0, and optionally wherein the composition is substantially free of an additional pH buffering component.

In embodiments, the disclosure provides a composition comprising: (a) an electrochemiluminescence (ECL) co-reactant selected from N-tert-butyldiethanolamine (tBDEA) methyldiethanolamine (MDEA), 3-[Bis-(2-hydroxy-ethyl)-amino]-propane-1-sulfonic acid (DEA-PS), and combination thereof; (b) an ionic component; and (c) a surfactant; wherein the composition has a pH of about 7.0 to about 8.0.

In embodiments, the disclosure provides composition that consist of or consist essentially of the recited components at the recited amounts. In compositions that consist essentially of the recited components, such compositions specifically exclude components that materially affect the ECL generating properties of the composition. The ECL generating properties of a composition can be determined by methods known to one of skill in the art. For example, the composition can be contacted with a known quantity of an ECL label on an electrode, and a voltage is applied to the electrode, thereby generating ECL. In embodiments, “materially unaffected” ECL generating properties means that a composition “consisting essentially of” the recited components generate about 80%, about 90%, about 95%, about 98%, about 99%, about 100%, about 101%, about 102%, about 105%, about 110%, or about 120% ECL as a composition “consisting of” the recited components. In embodiments, a composition that consists essentially of the recited components specifically excludes additional ECL-generating compounds, e.g., additional ECL co-reactants.

In embodiments, the disclosure provides a composition consisting essentially of: (a) triethanolamine (TEA); (b) an ionic component; and (c) a surfactant; wherein the composition has a pH of about 7.0 to about 8.0, and wherein the composition is substantially free of an additional pH buffering component. In embodiments, the disclosure provides a composition consisting of: (a) triethanolamine (TEA); (b) an ionic component; and (c) a surfactant; wherein the composition has a pH of about 7.0 to about 8.0, and wherein the composition is substantially free of an additional pH buffering component.

In embodiments, the disclosure provides a composition consisting essentially of: (a) triethanolamine (TEA); (b) an ionic component; (c) a surfactant; and (d) an ECL-labeled component, wherein the composition has a pH of about 7.0 to about 8.0, and wherein the composition is substantially free of an additional pH buffering component. In embodiments, the disclosure provides a composition consisting of: (a) triethanolamine (TEA); (b) an ionic component; (c) a surfactant; and (d) an ECL-labeled component, wherein the composition has a pH of about 7.0 to about 8.0.

In embodiments, the disclosure provides a composition consisting essentially of: (a) about 1000 mM to about 6500 mM of triethanolamine (TEA); (b) about 500 mM to about 2000 mM of an ionic component; and (c) a surfactant; wherein the composition has a pH of about 7.0 to about 8.0. In embodiments, the disclosure provides a composition consisting of: (a) about 1000 mM to about 6500 mM of triethanolamine (TEA); (b) about 500 mM to about 2000 mM of an ionic component; and (c) a surfactant; wherein the composition has a pH of about 7.0 to about 8.0.

In embodiments, the disclosure provides a composition consisting essentially of: (a) about 1000 mM to about 6500 mM of triethanolamine (TEA); (b) about 500 mM to about 2000 mM of an ionic component; (c) a surfactant; and (d) an ECL-labeled component, wherein the composition has a pH of about 7.0 to about 8.0. In embodiments, the disclosure provides a composition consisting of: (a) about 1000 mM to about 6500 mM of triethanolamine (TEA); (b) about 500 mM to about 2000 mM of an ionic component; (c) a surfactant; and (d) an ECL-labeled component, wherein the composition has a pH of about 7.0 to about 8.0.

In embodiments, the disclosure provides a composition consisting essentially of: (a) triethanolamine (TEA); (b) an ionic component; (c) an alkyl ether-polyethylene glycol (PEG); wherein the composition has a pH of about 7.0 to about 8.0. In embodiments, the disclosure provides a composition consisting of: (a) triethanolamine (TEA); (b) an ionic component; (c) an alkyl ether-polyethylene glycol (PEG); wherein the composition has a pH of about 7.0 to about 8.0.

In embodiments, the disclosure provides a composition consisting essentially of: (a) triethanolamine (TEA); (b) an ionic component; (c) an alkyl ether-PEG; and (d) an ECL-labeled component, wherein the composition has a pH of about 7.0 to about 8.0. In embodiments, the disclosure provides a composition consisting of: (a) triethanolamine (TEA); (b) an ionic component; (c) an alkyl ether-PEG; and (d) an ECL-labeled component, wherein the composition has a pH of about 7.0 to about 8.0.

In embodiments, the disclosure provides a composition consisting essentially of: (a) an electrochemiluminescence (ECL) co-reactant selected from N-tert-butyldiethanolamine (tBDEA), methyldiethanolamine (MDEA), 3-[Bis-(2-hydroxy-ethyl)-amino]-propane-1-sulfonic acid (DEA-PS), and combination thereof; (b) an ionic component; (c) a surfactant; and (d) a pH buffering component, wherein the composition has a pH of about 7.0 to about 8.0. In embodiments, the disclosure provides a composition consisting of: (a) an electrochemiluminescence (ECL) co-reactant selected from N-tert-butyldiethanolamine (tBDEA), methyldiethanolamine (MDEA), 3-[Bis-(2-hydroxy-ethyl)-amino]-propane-1-sulfonic acid (DEA-PS), and combination thereof; (b) an ionic component; (c) a surfactant; and (d) a pH buffering component, wherein the composition has a pH of about 7.0 to about 8.0.

In embodiments, the disclosure provides a composition consisting essentially of: (a) an ECL co-reactant selected from tBDEA, MDEA, DEA-PS, and combination thereof; (b) an ionic component; (c) a surfactant; (d) a pH buffering component, and (e) an ECL-labeled component, wherein the composition has a pH of about 7.0 to about 8.0. In embodiments, the disclosure provides a composition consisting of: (a) an ECL co-reactant selected from tBDEA, MDEA, DEA-PS, and combination thereof; (b) an ionic component; (c) a surfactant; (d) a pH buffering component, and (e) an ECL-labeled component, wherein the composition has a pH of about 7.0 to about 8.0.

As discussed herein, the ECL coreactants herein advantageously provide consistent ECL generation across different assay formats (e.g., washed and non-wash assays) and in combination with different classes of surfactants (e.g., mild surfactants that do not disrupt lipid bilayer membranes and harsher surfactants that can disrupt lipid bilayer membranes). Thus, compositions comprising the ECL coreactants herein (also referred to as “ECL read buffers”) are useful in a wide range of ECL-based binding assays.

In embodiments, the ECL coreactant comprises a tertiary amine. In embodiments, the ECL coreactant comprises a tertiary alkylamine. In embodiments, the ECL coreactant comprises a tertiary hydroxyalkylamine. In embodiments, the ECL coreactant comprises a zwitterionic tertiary amine. In embodiments, the ECL coreactant comprises a secondary amine. In embodiments, the ECL coreactant is tributylamine (TBA), (dibutyl)aminoethanol (DBAE), (diethyl)aminoethanol (DEAE), triethanolamine (TEA), butyldiethanolamine (BDEA), propyldiethanolamine (PDEA), ethyldiethanolamine (EDEA), methyldiethanolamine (MDEA), tert-butyldiethanolamine (tBDEA), dibutylamine (DBA), butylethanolamine (BEA), diethanolamine (DEA), dibutylamine propylsulfonate (DBA-PS), dibutylamine butylsulfonate (DB A-BS), butylethanolamine propylsulfonate (BEA-PS), butylethanolamine butylsulfonate (BEA-BS), diethanolamine propylsulfonate (also known as 3-[Bis-(2-hydroxy-ethyl)-amino]-propane-1-sulfonic acid; DEA-PS), or diethanolamine butylsulfonate (DEA-BS). Structures of exemplary ECL coreactants described herein are shown below.

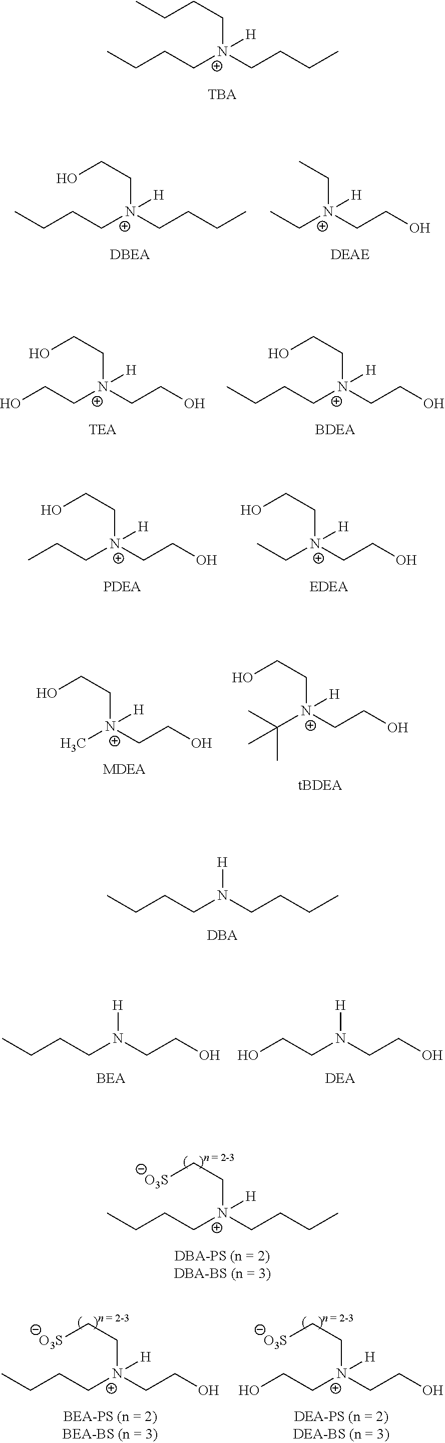

### Triethanolamine ECL Coreactant

The present disclosure provides compositions comprising an ECL coreactant. In embodiments, the ECL coreactant is triethanolamine (TEA). As discussed herein, it was discovered that TEA provides advantageous ECL generating properties in non-wash binding assays. In assays where the species of interest (e.g., analyte or binding complex as described herein) is captured and detected on a solid surface, TEA is capable of discriminating between “free” ECL labels that are not part of the species to be detected (e.g., analyte or binding complex) and “bound” ECL labels that are part of the species (e.g., analyte or binding complex) bound to the surface. Non-wash binding assays that utilize TEA as ECL coreactant have decreased non-specific ECL from the ECL label to the species to be detected, thereby decreasing the background ECL and increasing the signal-to-background ratio of the assay. In embodiments, a non-wash assay using TEA as ECL coreactant has a 2-fold higher, 3-fold higher, 4-fold higher, 5-fold higher, or 10-fold higher signal-to-background ratio as compared with an assay using tripropylamine (TPA) or piperazine-N,N′-bis(2-ethanesulfonic acid) (PIPES) as ECL coreactant. In embodiments, a non-wash assay using TEA as ECL coreactant has a 2-fold lower, 3-fold lower, 4-fold lower, 5-fold lower, 10-fold lower, 20-fold lower, or 40-fold lower limit of detection as compared with an assay using TPA or PIPES as ECL coreactant.

A further advantage of TEA as an ECL coreactant is the insensitivity of TEA to sample matrices and/or interferents. This is particularly beneficial in the context of non-wash assays, in which the reaction mixture may contain matrices from human or animal sources (e.g., containing proteins, cellular components and debris, culture media, and the like), which can also contain metabolite and/or drug interferents such as, e.g., acetaminophen, ibuprofen, naproxen, salicylic acid, and/or tolbutamine. In embodiments, TEA generates substantially the same ECL signal in a reaction mixture comprising one or more sample matrices and/or one or more interferents, as in a reaction mixture that does not comprise a sample matrix and/or an interferent.

It was further discovered that TEA provides the benefit of generating consistent ECL signal when used in the absence of surfactants or when combined with different types of surfactants, e.g., harsh and mild surfactants described herein. As used herein, a “harsh” surfactant is capable of disrupting, lysing and/or dissolving a lipid bilayer membrane (e.g., a membrane of a cell or an extracellular vesicle (EV)). In contrast, a “mild” surfactant does not disrupt, lyse or dissolve a lipid bilayer membrane. In embodiments, a composition comprising TEA and a harsh surfactant generates substantially similar ECL signal as a composition comprising identical components except that a mild surfactant is present instead of a harsh surfactant, when subjected to the same ECL-generating conditions (e.g., voltage waveform, type of electrode, amount of the composition, amount of ECL label, etc.). In embodiments, the harsh surfactant is TRITON™ X-100, TRITON™ X-114, NP-40, IGEPAL® CA-630, 3-[(3-cholamidopropyl)dimethylammonio]-1-propanesulfonate (CHAPS), or sodium dodecylsulfate (SDS). In embodiments, the mild surfactant is a BRIJ®, TWEEN®, PLURONIC®, or KOLLIPHOR® surfactant, or an alkyl ether-PEG surfactant such as PEG(18) tridecyl ether.

TEA has a pKa of about 7.7 and is capable maintaining the pH of a composition within about 7.0 to about 8.0, which is the typical desired pH range for biological assays. Moreover, TEA compositions having a pH of about 7.0 to about 8.0 preferentially generated ECL signal from an electrode-bound ECL label versus an unbound ECL label as described herein. Thus, compositions herein comprising TEA have the additional advantage of pH compatibility with biological assays and not requiring an additional pH buffering component, thereby simplifying the production process and lowering costs of the compositions. In embodiments, the composition comprising TEA is substantially free of an additional pH buffering component. Materials that can act as pH buffering components to maintain solutions within a specific pH range are known to one of skill in the art. For example, buffers that are capable of maintaining a pH of about 7.0 to about 8.0 include, but are not limited to, piperazine-N,N′-bis(2-ethanesulfonic acid) (PIPES), cholamine chloride, 3-(N-morpholino)propanesulfonic acid (MOPS), N-[tris(hydroxymethyl)methyl]-2-aminoethanesulfonic acid (TES), 3-(N,N-bis[2-hydroxyethyl]amino)-2-hydroxypropanesulfonic acid (DIPSO), 4-(N-morpholino)butanesulfonic acid (MOBS), acetamidoglycine, N-[tris(hydroxymethyl)methyl]-3-amino-2-hydroxypropanesulfonic acid (TAPSO), piperazine-1,4-bis(2-hydroxypropanesulfonic acid) dihydrate (POPSO), N-(hydroxyethyl)piperazine-N′-2-hydroxypropanesulfonic acid (HEPPSO), 3-[4-(2-hydroxyethyl)piperazin-1-yl]propane-1-sulfonic acid (HEPPS), tricine, glycinamide, N-(2-hydroxyethyl)piperazine-N′-(4-butanesulfonic acid) (HEPBS), and bicine. Further non-limiting examples of pH buffering components include tris(hydroxymethyl)aminomethane (“Tris”), phosphate, HEPES, glycylglycine (“GlyGly”), borate, acetate, and citrate. In embodiments, the composition comprising TEA does not comprise any of Tris, phosphate, HEPES, glycylglycine, borate, acetate, and citrate. In embodiments, the composition comprising TEA is substantially free of an additional component having a pKa of about 7.0 to about 8.0.

Moreover, many common pH buffering components have a tertiary amine in their structure and are capable of generating ECL. Exemplary pH buffering components that can act as an ECL coreactant are provided in U.S. Pat. No. 6,919,173 and include, but are not limited to, HEPES, POPSO, HEPPSO, and PIPES. In embodiments, the composition comprising TEA is substantially free of an additional ECL coreactant. In embodiments, the composition comprising TEA does not comprise any of HEPES, POPSO, HEPPSO, and PIPES. In embodiments, the composition comprising TEA does not comprise any of Tris, phosphate, HEPES, glycylglycine, borate, acetate, citrate, POPSO, HEPPSO, and PIPES.

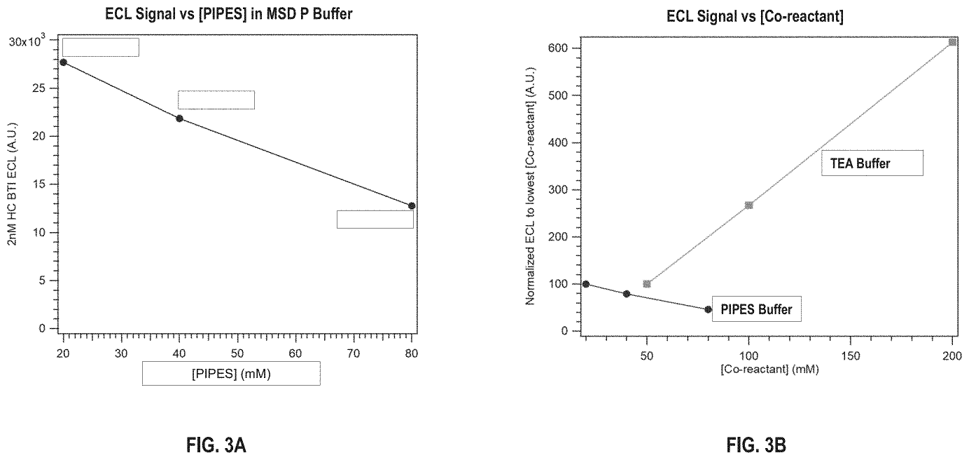

In embodiments, the concentration of TEA in the composition is about 500 mM to about 7000 mM, about 800 mM to about 6800 mM, about 1000 mM to about 6500 mM, about 1000 mM to about 6400 mM, about 1000 mM to about 6000 mM, about 1000 mM to about 5500 mM, about 1100 mM to about 5000 mM, about 1100 mM to about 4800 mM, about 1100 mM to about 4000 mM, about 1100 mM to about 3500 mM, about 1100 mM to about 3200 mM, about 1100 mM to about 3000 mM, about 1100 mM to about 2500 mM, about 1200 mM to about 2400 mM, or about 1200 mM to about 1600 mM. In embodiments, the concentration of TEA in the composition is about 1000 mM, about 1100 mM, about 1200 mM, about 1300 mM, about 1400 mM, about 1500 mM, about 1600 mM, about 1700 mM, about 1800 mM, about 1900 mM, about 2000 mM, about 2100 mM, about 2200 mM, about 2300 mM, about 2400 mM, about 2500 mM, about 2600 mM, about 2700 mM, about 2800 mM, about 2900 mM, about 3000 mM, about 3100 mM, about 3200 mM, about 3300 mM, about 3400 mM, about 3500 mM, about 3600 mM, about 3700 mM, about 3800 mM, about 3900 mM, about 4000 mM, about 4100 mM, about 4200 mM, about 4300 mM, about 4400 mM, about 4500 mM, about 4600 mM, about 4700 mM, about 4800 mM, about 4900 mM, about 5000 mM, about 5100 mM, about 5200 mM, about 5300 mM, about 5400 mM, about 5500 mM, about 5600 mM, about 5700 mM, about 5800 mM, about 5900 mM, about 6000 mM, about 6100 mM, about 6200 mM, about 6300 mM, about 6400 mM, about 6500 mM, about 6600 mM, about 6700 mM, about 6800 mM, about 6900 mM, or about 7000 mM. In embodiments, the concentration of TEA in the composition is at least about 1000 mM, at least about 1200 mM, at least about 1600 mM, at least about 1800 mM, at least about 2000 mM, at least about 2500 mM, at least about 3000 mM, at least about 3500 mM, at least about 4000 mM, at least about 4500 mM, at least about 5000 mM, at least about 5500 mM, or at least about 6000 mM. Surprisingly, TEA concentration in the composition showed a positive correlation with strength of the generated ECL signal, which was unexpected as other ECL coreactants such as PIPES (which was expected to behave similarly to TEA, as PIPES and TEA both have the ability to confine ECL near the electrode as described herein) have shown decrease in ECL generation with increasing ECL coreactant concentration (see, e.g., FIGS. 3A and 3B). Thus, the TEA compositions provided herein are capable of preferentially and consistently generating ECL signal from an electrode-bound ECL label versus an unbound ECL label as described herein, over a broad concentration range, e.g., from about 1000 mM to about 6500 mM. The consistency of electrode-bound ECL generation decreases variability in ECL generation in ECL-based assays, e.g., in washed or non-washed assay formats. An ECL coreactant that can be used at a high concentration, e.g., TEA, provides advantages in non-wash assays by minimizing dilution of the sample and/or assay mixture, avoiding perturbation of the binding equilibrium and kinetics of the assay components, and therefore maximizing ECL signal. An ECL coreactant that can be used at high concentrations, e.g., TEA, are also useful in assays with lower affinity binding and/or detection reagents, providing improved sensitivity as compared with ECL coreactants that cannot be used at high concentrations (e.g., PIPES).

### Alkyl Diethanolamine/Zwitterionic Tertiary Amine ECL Coreactant

The present disclosure further provides compositions comprising an alkyl diethanolamine ECL coreactant and/or a zwitterionic tertiary amine ECL coreactant. In embodiments, the alkyl diethanolamine is butyldiethanolamine (BDEA), propyldiethanolamine (PDEA), ethyldiethanolamine (EDEA), methyldiethanolamine (MDEA), or tert-butyldiethanolamine (tBDEA). In embodiments, the alkyl diethanolamine is N-tert-butyldiethanolamine (tBDEA) or methyldiethanolamine (MDEA). In embodiments, the zwitterionic tertiary amine ECL coreactant is dibutylamine propylsulfonate (DBA-PS), dibutylamine butylsulfonate (DBA-BS), butylethanolamine propylsulfonate (BEA-PS), butylethanolamine butylsulfonate (BEA-BS), diethanolamine propylsulfonate (also known as 3-[Bis-(2-hydroxy-ethyl)-amino]-propane-1-sulfonic acid; DEA-PS), or diethanolamine butylsulfonate (DEA-BS). In embodiments, the zwitterionic tertiary amine ECL coreactant is DEA-PS. It was discovered that tBDEA, MDEA, and DEA-PS advantageously show consistent ECL generating properties when used in the absence of surfactant or when combined with different types of surfactants, e.g., harsh and mild surfactants described herein. In embodiments, a composition comprising tBDEA, MDEA, and/or DEA-PS and a harsh surfactant generates substantially similar ECL signal as a composition comprising identical components except that a mild surfactant is present instead of a harsh surfactant, when subjected to the same ECL-generating conditions (e.g., voltage waveform, type of electrode, amount of the composition, amount of ECL label, etc.). In embodiments, the harsh surfactant is TRITON™ X-100, TRITON™ X-114, NP-40, IGEPAL® CA-630, 3-[(3-cholamidopropyl)dimethylammonio]-1-propanesulfonate (CHAPS), or sodium dodecylsulfate (SDS). In embodiments, the mild surfactant is a BRIJ®, TWEEN®, PLURONIC®, or KOLLIPHOR® surfactant, or an alkyl ether-PEG surfactant such as PEG(18) tridecyl ether.

In embodiments, the concentration of the alkyl diethanolamine or the zwitterionic tertiary amine in the composition is about 10 mM to about 500 mM, about 20 mM to about 400 mM, about 50 mM to about 250 mM, or about 100 mM to about 200 mM. In embodiments, the concentration of the alkyl diethanolamine the zwitterionic tertiary amine in the composition is about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM, or about 500 mM. In embodiments, the alkyl diethanolamine is tBDEA. In embodiments, the alkyl diethanolamine is MDEA. In embodiments, the alkyl diethanolamine is a combination of tBDEA and MDEA. In embodiments, the zwitterionic tertiary amine is DEA-PS. In embodiments, the composition comprises a combination of two or more of tBDEA, MDEA, and DEA-PS.

In embodiments, the composition comprising the alkyl diethanolamine and/or zwitterionic tertiary amine (e.g., tBDEA, MDEA, and/or DEA-PS), further comprises a pH buffering component. In embodiments, the pH buffering component has a pKa of about 7.0 to about 8.0. In embodiments, the pH buffering component is capable of maintaining pH of the composition at about 7.0 to about 8.5, about 7.2 to about 8.0, or about 7.4 to about 7.9. In embodiments, the pH buffering component comprises Tris, phosphate, HEPES, glycylglycine, borate, acetate, citrate, PIPES, MOPS, TES, DIPSO, MOBS, TAPSO, POPSO, HEPPSO, HEPPS, tricine, glycinamide, HEPBS, bicine, or a combination thereof. In embodiments, the pH buffering component is Tris, phosphate, HEPES, glycylglycine, borate, acetate, citrate, or a combination thereof. In embodiments, the pH buffering component comprises Tris. In embodiments, the pH buffering component comprises phosphate.

In embodiments, the concentration of the pH buffering component in the composition is about 10 mM to about 800 mM, about 20 mM to about 600 mM, about 50 mM to about 400 mM, about 100 mM to about 300 mM, about 120 mM to about 280 mM, or about 150 mM to about 250 mM. In embodiments, the concentration of the pH buffering component in the composition is about 50 mM, about 100 mM, about 150 mM, about 200 mM, about 250 mM, about 300 mM, about 350 mM, about 400 mM, about 450 mM, or about 500 mM.

### Ionic Component

In embodiments, the compositions herein comprise an ionic component. Ionic components, such as salts, dissociate into ions in solution. It was discovered that high ion concentrations can advantageously reduce non-specific binding of an ECL label with the ECL coreactant. Non-limiting examples of ionic components include salts comprising the cations Li⁺, Na⁺, K⁺, Rb⁺, Cs⁺, Mg⁺², Ca⁺², and NH₄⁺, and/or salts comprising the anions F⁻, Cl⁻, Br⁻, K⁻. phosphate, sulfate, and borate. In embodiments, the ionic component comprises Li⁺, Na⁺, or K⁺. In embodiments, the ionic component comprises Cl⁻. In embodiments, the ionic component comprises lithium chloride (LiCl), sodium chloride (NaCl), potassium chloride (KCl), or a combination thereof. In embodiments, the ionic component comprises NaCl. In embodiments, the ionic component comprises KCl.

In embodiments, the concentration of the ionic component in the composition is about 100 mM to about 2000 mM, about 200 mM to about 1800 mM, about 300 mM to about 1700 mM, about 400 mM to about 1600 mM, about 500 mM to about 1500 mM, about 600 mM to about 1200 mM, about 700 mM to about 1000 mM, or about 800 mM to about 900 mM. In embodiments, the concentration of the ionic component in the composition is about 500 mM, about 550 mM, about 600 mM, about 650 mM, about 700 mM, about 750 mM, about 800 mM, about 850 mM, about 900 mM, about 950 mM, about 1000 mM, about 1100 mM, about 1200 mM, about 1300 mM, about 1400 mM, or about 1500 mM.

In embodiments, the composition comprises about 100 mM to about 2000 mM, about 200 mM to about 1800 mM, about 300 mM to about 1700 mM, about 400 mM to about 1600 mM, about 500 mM to about 1500 mM, about 600 mM to about 1200 mM, about 700 mM to about 1000 mM, or about 800 mM to about 900 mM NaCl. In embodiments, the composition comprises about 100 mM to about 2000 mM, about 200 mM to about 1800 mM, about 300 mM to about 1700 mM, about 400 mM to about 1600 mM, about 500 mM to about 1500 mM, about 600 mM to about 1200 mM, about 700 mM to about 1000 mM, or about 800 mM to about 900 mM KCl. In embodiments, the composition comprises about 100 mM to about 2000 mM, about 200 mM to about 1800 mM, about 300 mM to about 1700 mM, about 400 mM to about 1600 mM, about 500 mM to about 1500 mM, about 600 mM to about 1200 mM, about 700 mM to about 1000 mM, or about 800 mM to about 900 mM LiCl.

In embodiments, the composition has an ionic strength of about 0.2 M to about 2 M, about 0.5 M to about 1.5 M, about 0.75 M to about 1.25 M, or about 0.8 M to about 1.0 M. In embodiments, the composition has an ionic strength of greater than or about 0.3 M, greater than or about 0.5 M, greater than or about 0.8 M, or greater than or about 1.0 M. In embodiments, the composition comprises chloride ion and the concentration of the chloride ion is greater than or about 0.3 M, greater than or about 0.5 M, greater than or about 0.8 M, or greater than or about 1.0 M.

In embodiments, non-specific binding (NSB) in an immunoassay with ECL as the assay readout is lower with the composition containing the ionic component as compared to an otherwise identical composition containing no ionic component.

### Surfactant

It was unexpectedly discovered that when using compositions provided herein, e.g., comprising TEA, tBDEA, MDEA and/or DEA-PS as ECL coreactant, the ECL generating properties of the composition are substantially unaffected by the presence, concentration, or structure of surfactants in the composition. In contrast, TPA-based compositions generally require the presence of surfactants for optimal signal generation. In particular, TPA provides optimal ECL generation in the presence of surfactants comprising aromatic moieties, such as the phenolic ether moiety in TRITON™ X-100.

In embodiments, the compositions herein are substantially free of a surfactant. In embodiments, the compositions herein comprise a surfactant. In embodiments, the compositions herein comprise a surfactant at a concentration below the critical micellar concentration (CMC) of the surfactant. The CMC is the concentration of surfactants above which micelles form, and any additional amount of surfactant added to the composition above the CMC are incorporated into the micelles. The CMC of a surfactant can be determined by one of skill in the art, e.g., using a titration method as described in Wu et al., Anal Chem 92(6):4259-4265 (2020), and/or using devices such as a dynamic contact angle measuring device and/or a tensiometer.

In embodiments, the compositions herein comprise a non-ionic surfactant. In embodiments, the compositions herein comprise an ionic surfactant. Non-ionic surfactants include the surfactant classes known by the trade names of NONIDET™ (octylphenoxypolyethoxyethanol), BRIJ® (polyoxyethylene fatty ether), TRITON™ (2-[4-(2,4,4-trimethylpentan-2-yl)phenoxy]ethanol), TWEEN® (polysorbate), KOLLIPHOR® (polyoxyl castor oil), THESIT® (polyethylene glycol dodecyl ether), LUBROL® (polyoxyethylene alkyl ether), GENAPOL® (iso-tridecyl alcohol polyglycol ether), PLURONIC® (poloxamer block copolymers of poly(ethylene oxide) (PEO) and poly(propylene oxide) (PPO) arranged as PEO-PPO-PEO), TETRONIC® (poloxamine block copolymers of PEO-PPO), SYNPERONIC® (block copolymer of poly(ethylene glycol) (PEG) and poly(propylene glycol) (PPG) arranged as PEG-PPG-PEG), and SPAN® (sorbitan). Specific examples of non-ionic surfactants include, e.g., KOLLIPHOR® P-407 (PEG₁₀₁-PPG₅₆-PEG₁₀₁; also known as Poloxamer 407), PLURONIC® P-123 (PEO₁₈-PPO₇₂-PEO₁₈), PLURONIC® L-121 (PEG₅-PPG₆₈-PEG₅), PLURONIC® 31R1 (PPO₂₆-PEO₅-PPO₂₆), TETRONIC® 701 (ethylenediamine tetrakis(propoxylate-block-ethoxylate) tetrol), BRIJ® L4 (polyethylene glycol dodecyl ether), BRIJ® 58 (polyethylene glycol hexadecyl ether), TWEEN® 20 (polysorbate 20), 2,4,7,9-tetramethyl-d-decyne-4,7-diol ethoxylate, and alkyl ether-polyethylene glycols (PEG) such as PEG(10) tridecyl ether, PEG(12) tridecyl ether, and PEG(18) tridecyl ether).

In embodiments, the surfactant comprises a phenol ether. In embodiments, the surfactant is 2-[4-(2,4,4-trimethylpentan-2-yl)phenoxy]ethanol (TRITON™ X-100). In the context of surfactants described herein, TRITON™ X-100 is a “harsh” surfactant that is capable of disrupting, lysing and/or dissolving a lipid bilayer membrane, e.g., a membrane of a cell or an extracellular vesicle (EV).

In embodiments, the surfactant does not comprise an aromatic moiety. In embodiments, the surfactant does not comprise a phenol ether. In embodiments, the surfactant does not disrupt, lyse or dissolve a lipid bilayer membrane, e.g., a membrane of a cell or an extracellular vesicle (EV). Such surfactants can be referred to as “mild” surfactants. Examples of mild surfactants include the surfactant classes known by the trade names BRIJ®, TWEEN®, PLURONIC®, or KOLLIPHOR®. In embodiments, the surfactant does not comprise an ester linkage. In embodiments, the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is an alkyl ether-polyethylene glycol (PEG). In embodiments, the alkyl ether-polyethylene glycol (PEG) is PEG(10) tridecyl ether, PEG(12) tridecyl ether, PEG(18) tridecyl ether, or a combination thereof. In embodiments, the surfactant is PEG(18) tridecyl ether.

As described herein, the compositions herein advantageously provide consistent ECL signal generation in the presence of different types of surfactants, e.g., harsh and mild surfactants described herein. In embodiments, a composition comprising TEA, tBDEA, MDEA, DEA-PS, or a combination thereof and a harsh surfactant generates substantially similar ECL signal as a composition comprising identical components except that a mild surfactant is present instead of a harsh surfactant, when subjected to the same ECL-generating conditions (e.g., voltage waveform, type of electrode, amount of the composition, amount of ECL label, etc.). In embodiments, a composition comprising TEA, BDEA, tBDEA, MDEA, DEA-PS, or a combination thereof and a harsh surfactant generates substantially similar ECL signal as a composition comprising identical components except that a mild surfactant is present instead of a harsh surfactant, when subjected to the same ECL-generating conditions (e.g., voltage waveform, type of electrode, amount of the composition, amount of ECL label, etc.). In embodiments, the harsh surfactant is TRITON™ X-100. In embodiments, the mild surfactant is a BRIJ®, TWEEN®, PLURONIC®, or KOLLIPHOR® surfactant, or an alkyl ether-PEG surfactant such as PEG(18) tridecyl ether.

In embodiments, the concentration of the surfactant in the composition is such that the composition has an air-liquid surface tension of less than or about 50 dyne/cm, less than or about 40 dyne/cm or less than or about 35 dyne/cm. In embodiments, the surfactant is present in the composition at its cmc, greater than or about two times its cmc, or greater than or about five times its cmc.

In embodiments, the surfactant is about 0.1% (v), about 0.5% (v/v), about 1% (v/v), about 2% (v/v), about 5% (v/v), about 7% (v/v), or about 10% (v/v) of the composition. In embodiments, the concentration of the surfactant in the composition is about 0.1 mM to about 20 mM, about 0.1 mM to about 10 mM, about 0.5 mM to about 8 mM, about 0.75 mM to about 6 mM, or about 1 mM to about 5 mM. In embodiments, the concentration of the surfactant in the composition is about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, or about 10 mM.

In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM 2-[4-(2,4,4-trimethylpentan-2-yl)phenoxy]ethanol (TRITON™ X-100). In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM Poloxamer 407 (KOLLIPHOR® P-407). In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM PEO₁₈-PPO₇₂-PEO₁₈ (PLURONIC® P-123). In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM PEG₅-PPG₆₈-PEG₅(PLURONIC® L-121). In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM PPO₂₆-PEO₅-PPO₂₆ (PLURONIC®31R1). In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM ethylenediamine tetrakis(propoxylate-block-ethoxylate) tetrol (TETRONIC® 701). In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM polyethylene glycol dodecyl ether (BRIJ® L4). In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM polyethylene glycol hexadecyl ether (BRIJ® 58). In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM polysorbate 20 (TWEEN® 20). In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM 2,4,7,9-tetramethyl-d-decyne-4,7-diol ethoxylate. In embodiments, the composition comprises about 0.1 mM to about 20 mM, about 0.1 mM to about 10 mM, about 0.5 mM to about 8 mM, about 0.75 mM to about 6 mM, or about 1 mM to about 5 mM alkyl ether-PEG. In embodiments, the alkyl ether-PEG is PEG(18) tridecyl ether.

pH

In embodiments, the compositions herein have a pH of about 6.0 to about 9.0, pH of about 7.0 to about 8.0, pH of about 7.2 to about 7.6, pH of about 7.5 to about 7.8, pH of about 7.4 to about 7.9, pH of about 7.0, about 7.1, about 7.2, about 7.3, about 7.4, about 7.5, about 7.6, about 7.7, about 7.8, about 7.9, or about 8.0. In embodiments, the pH of the composition is about 7.5. In embodiments, the pH of the composition is about 7.8.

In embodiments, the composition comprising TEA has a pH of about 7.0 to about 8.0, about 7.4 to about 7.9, or about 7.5 to about 7.8, and is substantially free of an additional pH buffering component. In embodiments, the composition comprising TEA has a pH of about 7.0 to about 8.0, about 7.4 to about 7.9, or about 7.5 to about 7.8, and is substantially free of an additional component having a pKa of about 7.0 to about 8.0. In embodiments, the composition comprising TEA does not comprise any of Tris, phosphate, HEPES, glycylglycine, borate, acetate, and citrate. In embodiments, the composition comprising TEA does not comprise any of HEPES, POPSO, HEPPSO, and PIPES. In embodiments, the composition comprising TEA does not comprise any of Tris, phosphate, HEPES, glycylglycine, borate, acetate, citrate, POPSO, HEPPSO, and PIPES.

### ECL-Labeled Component

In embodiments, the compositions herein comprise an ECL-labeled component. In embodiments, the ECL coreactant composition provided herein, e.g., comprising TEA, tBDEA, MDEA and/or DEA-PS and the ECL-labeled component, is capable of generating ECL. In embodiments, the ECL-labeled component comprises an ECL label. In embodiments, the ECL-labeled component comprises a detection reagent. In embodiments, the ECL-labeled component comprises a binding partner of a detection reagent.

In embodiments, ECL-labeled component is a detection reagent that comprises an ECL label. In embodiments, the detection reagent comprises an antibody or antigen-detection fragment thereof, antigen, ligand, receptor, oligonucleotide, hapten, epitope, mimotope, or aptamer. In embodiments, the detection reagent is an antibody or a variant thereof, including an antigen/epitope-detection portion thereof, an antibody fragment or derivative, an antibody analogue, an engineered antibody, or a substance that binds to antigens in a similar manner to antibodies. In embodiments, the detection reagent comprises at least one heavy or light chain complementarity determining region (CDR) of an antibody. In embodiments, the detection reagent comprises at least two CDRs from one or more antibodies. In embodiments, the detection reagent is an antibody or antigen-detection fragment thereof. In embodiments, the detection reagent is covalently linked to the ECL label via a conjugation linker. Methods of conjugating labels, e.g., ECL labels, to detection reagents are known to one of ordinary skill in the art.

In embodiments, the ECL-labeled component is a binding partner of a detection reagent. In embodiments, the ECL-labeled component and the detection reagent form a complex that is capable of being detected by ECL. In embodiments, the ECL-labeled component and the detection reagent comprise a receptor-ligand pair, an antigen-antibody pair, a hapten-antibody pair, an epitope-antibody pair, a mimotope-antibody pair, an aptamer-target molecule pair, or an intercalator-target molecule pair. In embodiments, the ECL-labeled component and the detection reagent comprise complementary oligonucleotides. In embodiments, the ECL-labeled component and the detection reagent comprise a biotin-avidin or biotin-streptavidin pair.

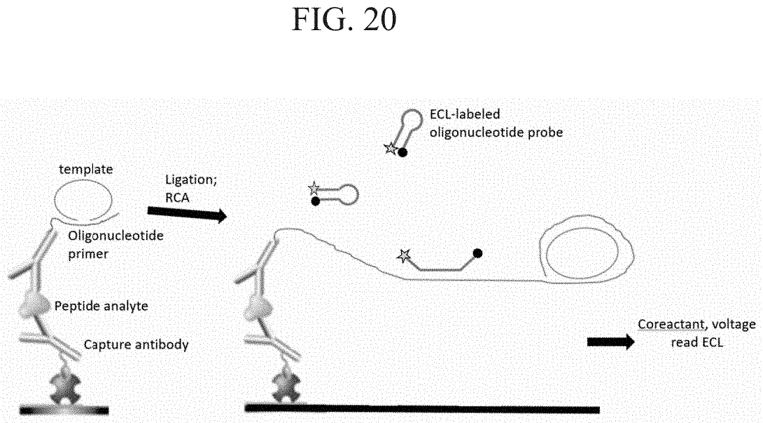

In embodiments, the ECL-labeled component is an oligonucleotide. In embodiments, the ECL-labeled oligonucleotide and the detection reagent comprise complementary oligonucleotides, and in other embodiments the ECL-labeled oligonucleotide is the detection reagent. In embodiments, the binding reagent comprises an oligonucleotide, and the ECL-labeled oligonucleotide is complementary to that oligonucleotide. In embodiments, the binding partner comprises an oligonucleotide, and the ECL-labeled oligonucleotide is complementary to that binding partner oligonucleotide. In embodiments, the binding partner is an analyte oligonucleotide, and the ECL-labeled oligonucleotide is complementary to the analyte oligonucleotide. In embodiments, the binding partner of the ECL-labeled oligonucleotide is an analyte oligonucleotide, the ECL-labeled oligonucleotide is complementary to a portion of the the analyte oligonucleotide and the binding reagent is a capture oligonucleotide that is complementary to a different portion of the analyte oligonucleotide. In embodiments, the ECL-labeled component is an oligonucleotide probe that is complementary to an extended primer that is attached to a detection reagent (e.g., a detection antibody) that binds to a target analyte such as a peptide or protein. In embodiments, the binding reagent is streptavidin the binding complex comprising an oligonucleotide is a complex of biotinylated capture antibody, peptide, and second antibody comprising an oligonucleotide primer (see, for example but not limited to, FIG. 20).

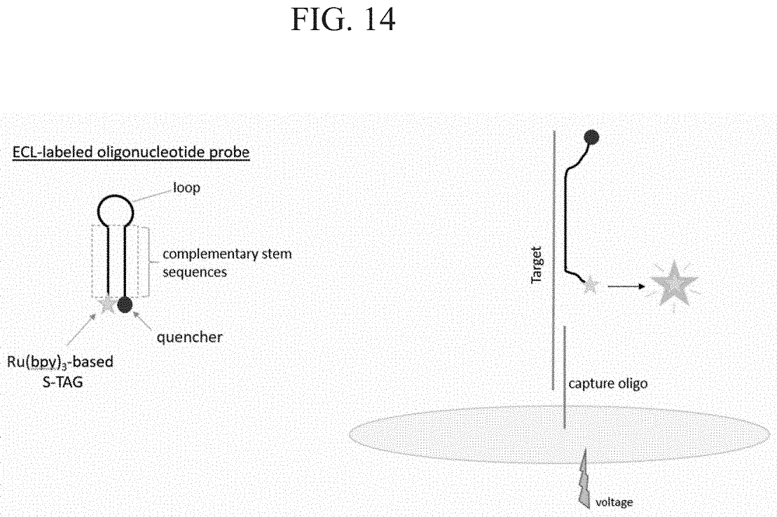

In embodiments, the ECL-labeled oligonucleotide is a probe. In embodiments, the ECL-labeled oligonucleotide is a probe that is 10, 11, 12, 13, 14, 15, 16, 17, 18, 19, 20, 21, 22, 23, 24, 25, 26, 27, 28, 29, 30, 31, 32, 33, 34, 35, 36, 37, 38, or more nucleotides long, or a range defined by any two of the preceding values. In an embodiment, the ECL-labeled oligonucleotide probe comprises a stem-loop or hairpin structure, an ECL label, and a quenching moiety, wherein said quenching moiety is in proximity to the ECL label and quenches the ECL label when the ECL-labeled oligonucleotide probe is in a stem-loop or hairpin configuration, but does not quench the ECL label when the stem-loop or hairpin structure is in an open configuration. In embodiments, the ECL-labeled oligonucleotide probe with the stem-loop or hairpin structure is an ECL-labeled molecular beacon probe. In embodiments, the binding partner of the ECL-labeled hairpin or molecular beacon oligonucleotide probe is an analyte oligonucleotide, the ECL-labeled oligonucleotide probe is complementary to a portion of the the analyte oligonucleotide and the binding reagent is a capture oligonucleotide that is complementary to a different portion of the analyte oligonucleotide (see, for example but not limited to, FIG. 14). In embodiments, the ECL-labeled molecular beacon probe is complementary to an extended primer that is attached to a detection reagent (e.g., a detection antibody) that binds to a target analyte such as a peptide or protein (see, for example but not limited to, FIG. 20).

ECL-labeled molecular beacon probes can be made using conventional methods know in the art for conventional molecular beacon probes. In embodiments, probes can have a 10, 11, 12, 13, 14, 15, 16, 17, 18, 19, 20, 21, 22, 23, 24, 25, 26, 27, 28, 29, 30 base long probe sequence, or a range defined by any two of the preceding values, for example a 10 to 30, 10 to 15, or 15 to 30-base probe sequence and a 5, 6, 7, 8, 9, 10, 11, 12, 13 base long 5′ and 3′ stem complement sequences, or a range defined by any two of the preceding values, for example a 5 to 8-base 5′ and 3′ stem complement sequences. In embodiments, the stems are 5-8 basepairs long and have a very high GC content (75 to 100 percent). In embodiments, the ECL-labeled molecular beacon probes are labeled at the 5′ and 3′ ends, with a ECL label and a quenching moiety, for example a 5′ ECL label and a 3′ quenching moiety, or vice versa. Design tools for conventional molecular beacon probes can be found at www.molecular-beacons.org/MB_SC_design. In embodiments, the use of short probe sequences can reduce the need for the use of a stem-loop structure, allowing more effective static quenching via contact. To achieve these goals molecular beacons based on LNA sequences have proved valuable. Examples are found in Eboigbodin K E, et al. Rapid and sensitive real-time assay for the detection of respiratory syncytial virus using RT-SIBA, BMC Infect Dis. 2017, which is incorporated herein by reference in its entirety. An example of such a probe is /56-ROXN/+CA+A+TA+T+T+GA+GA+TA/3IABkFQ/. In embodiments, ECL-labeled oligonucleotide probes may be designed using a duplex stabilizing technology (Minor Groove Binder, MGB™), BHQplus® Probes (Biosearch Technology), to allow shorter probe designs. Minor Groove Binders are available as phosphoramidites, and CPG allowing their inclusion into the oligonucleotide synthetic process during probe synthesis (IDT). An embodiment is to use the MGB-CPG support, add a quencher molecule followed by the sequence and a final 5′ amino group for labeling with an ECL moiety. Alternatively, they may be added post synthesis via the use of the NHS-ester based labeling of amino groups introduced during oligonucleotide synthesis. Minor Groove Binders include those described in U.S. Pat. No. 9,334,495, which incorporated herein by reference in its entirety.

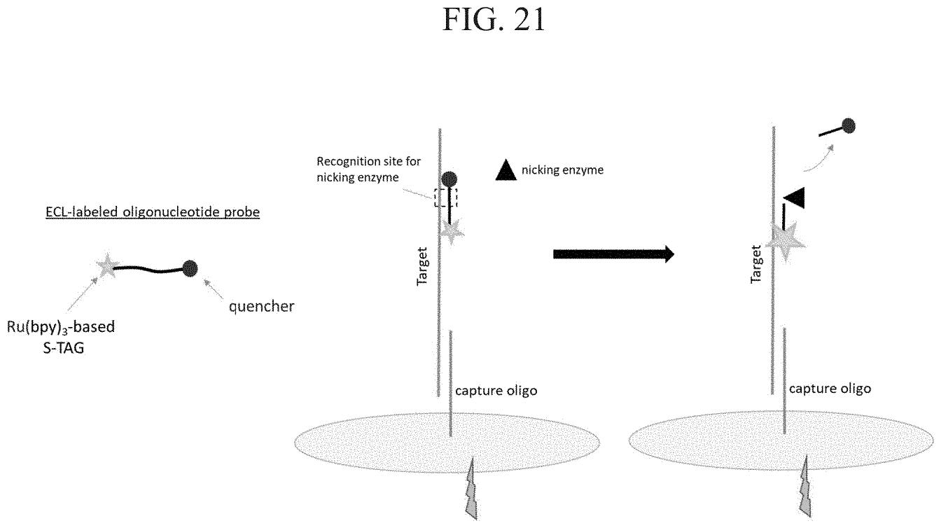

In embodiments the ECL-labeled oligonucleotide probes comprise an ECL label and a quencher that are sufficiently close to each other on the probe when the probe is in a linear configuration (e.g., not in a stem-loop or hairpin configuration) that the quenching moiety quenches the ECL signal whether the probe is hybridized to a complementary oligonucleotide or not (e.g., as illustrated in FIG. 21). Such ECL-labeled probes can be made using conventional methods know in the art, for example those used to design TaqMan® probes. In embodiments, the ECL-labeled oligonucleotide probe that is 10, 11, 12, 13, 14, 15, 16, 17, 18, 19, 20, 21, 22, 23, 24, 25, 26, 27, 28, 29, 30, 31, 32, 33, 34, 35, 36, 37, 38, or more nucleotides long, or a range defined by any two of the preceding values. In embodiments, the ECL-labeled probes are labeled at the 5′ and 3′ ends, with a ECL label and a quenching moiety, for example a 5′ ECL label and a 3′ quenching moiety, or vice versa. In embodiments, one or both of the ECL label and quenching moiety are attached at a position other than the 5′ or 3′ end of the oligonucleotide. In embodiments, the quenching moiety is at the 5′ end of the oligonucleotide.

In embodiments, the ECL-labeled oligonucleotide probe is DNA, RNA, a mixture of both, and/or comprises one or more modified nucleic acids. In embodiments, the ECL-labeled oligonucleotide comprises one or more nucleotides that are resistant to enzymatic cleavage, for example by an exonuclease or an endonuclease. In embodiments, the resistant nucleotide(s) are peptide nucleic acids, or other resistant nucleic acid mimic known in the art. In embodiments, only a portion of the ECL-labeled oligo nucleotide probe is resistant, and the quenching moiety is on a portion (e.g. a 5′ portion) that is not resistant, while the ECL label is on a portion (e.g., a central and/or 3′ portion) that is resistant, so that when an enzyme (e.g., a 5′ exonuclease) cleaves the ECL-labeled oligonucleotide, the quenching moiety is released into the solution and the resistant portion of the ECL-labeled probe comprising the ECL label remains hybridized to the complementary oligonucleotide (e.g., the oligonucleotide of the binding partner and/or binding complex). In embodiments, the ECL-labeled oligonucleotide probe comprises a feature, for example but not limited to, a sequence and/or type of nucleotide (e.g., RNA), that is recognized by an enzyme which cleaves the ECL-labeled oligonucleotide probe only when it is hybridized to a complementary oligonucleotide (e.g. the oligonucleotide of the binding partner and/or binding complex) (e.g., as illustrated in FIG. 21). In embodiments, the enzyme is a restriction endonuclease, optionally a nicking endonuclease, and the feature is a sequence recognized by the restriction endonuclease, or the enzyme is an RNasH2 enzyme, and the feature is one or more RNA nucleotides. In embodiments, the ECL-labeled oligonucleotide probe comprises the feature near to the quenching moiety so that when the enzyme cleaves the ECL-labeled oligonucleotide probe, the quenching moiety is released into the solution and the ECL-labeled probe comprising the ECL label remains hybridized to the complementary oligonucleotide (e.g., the oligonucleotide of the binding partner and/or binding complex).

In embodiments, a plurality of ECL-labeled oligonucleotide probes can be used. In embodiments, multiple ECL-labeled oligonucleotide probes, each having different sequences complementary to sequences of different target oligonucleotides (e.g., the oligonucleotides on binding reagents, detection reagents, binding partners, and/or oligonucleotide analytes), allowing specific hybridization of ECL-labeled oligonucleotide probes to their respective target oligonucleotides. In some embodiments, the target oligonucleotides are localized to known positions on a substrate, such that the presence of an ECL signal at the known position can be correlated to the presence of the target oligonucleotide (e.g., the oligonucleotides on binding reagents, detection reagents, binding partners, and/or oligonucleotide analytes). In some embodiments, multiple different target oligonucleotides are localized to multiple spots on the bottom surface of a culture plate well, wherein each target oligonucleotide having a specific sequences is localized to a specific, predefined spot. In some embodiments, each spot contains a specific, immobilized binding reagent (for example, a capture oligonucleotide) that is different from the binding reagents immobilized to the other spots on the substrate surface (e.g., a plate well bottom). Each spot may be coated with an electrode layer on which the binding reagent is immobilized. In this way, a multiplexing reaction can be performed using the multiple ECL-labeled oligonucleotide probes. In some embodiments, the multiplex reaction utilizes 2, 3, 4, 5, 6, 7, 8, 9, 10, 11, 12, 13, 14, 15 or more different ECL-labeled oligonucleotide probes, each having a different oligonucleotide sequence complementary to a different target oligonucleotide. In embodiments, the ECL label is a label described herein, for example in the paragraph below. In embodiments, the quencher moiety is selected from the group consisting of ATTO 540Q, ATTO 575Q, ATTO 580Q, ATTO 612Q, Iowa Black FQ, Iowa Back RQ, QSY 21, IRDye QC-1, BHQ0, BHQ1, BHQ-2, BHQ-3, Dabcyl, QSY 7, QSY 9, QSY 21, QSY 35, QXL 490, QXL 520, QXL 570, and QXL 670. In embodiments, the quencher moiety is an anti-ECL label antibody, or binding fragment thereof. In embodiments, the quenching moiety is ferrocene or iron tris-bipyridine. In embodiments, the ECL label has Formula II below. In embodiments, the ECL label has Formula II below, and the quencher moiety is BHQ2. In embodiments, the ECL label has Formula II below, and the quencher moiety is Iowa Black. In embodiments, the ECL label has Formula II below, and the quencher moiety is Dabcyl.

In embodiments, the ECL label comprises an electrochemiluminescent organometallic complex. In embodiments, the electrochemiluminescent organometallic complex comprises ruthenium, osmium, iridium, rhenium, and/or a lanthanide metal. In embodiments, the ECL label comprises ruthenium. In embodiments, the electrochemiluminescent organometallic complex comprises a substituted or unsubstituted bipyridine or a substituted or unsubstituted phenanthroline. In embodiments, the ECL label comprises a substituted bipyridine. In embodiments, the ECL label comprises ruthenium (II) tris-bipyridine. In embodiments, the ECL label comprises an organometallic complex comprising at least one substituted bipyridine ligand, wherein the substituted bipyridine ligand comprises at least one sulfonate group. In embodiments, the ECL label comprises an organometallic complex comprising at least two substituted bipyridine ligands, wherein each substituted bipyridine ligand comprises at least one sulfonate group. In embodiments, the substituted bipyridine ligand comprising at least one sulfonate group is a compound of Formula I:

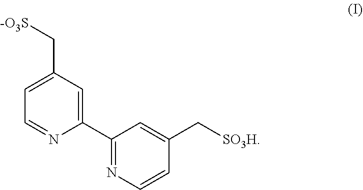

In embodiments, the ECL label comprises three ligands, wherein a first ligand is a compound of Formula I, and wherein a second ligand comprises a bipyridine having at least one substituent that is covalently linked to the detection reagent. In embodiments, the ECL label comprises an organometallic complex that comprises three ligands, wherein two of the ligands are each a compound of Formula I, and wherein the third ligand comprises a bipyridine having at least one substituent that is covalently linked to the detection reagent.

In embodiments, the first detectable label is a compound of Formula II:

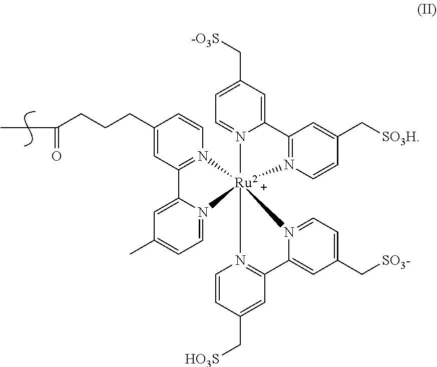

Further exemplary ECL labels can be found in U.S. Pat. Nos. 5,714,089; 6,136,268; 6,316,607; 6,468,741; 6,479,233; 6,808,939; and 9,499,573, each of which are herein incorporated by reference in its entirety.

### TEA Compositions

In embodiments, the disclosure provides a composition comprising about 1000 mM to about 6500 mM TEA and about 500 mM to about 1500 mM ionic component, wherein the composition has a pH of about 7.0 to about 8.0; and wherein the ionic component is NaCl, KCl, or LiCl. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1100 mM to about 3500 mM TEA and about 600 mM to about 1200 mM ionic component, wherein the composition has a pH of about 7.0 to about 8.0; and wherein the ionic component is NaCl, KCl, or LiCl. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM to about 1600 mM TEA and about 700 mM to about 900 mM ionic component, wherein the composition has a pH of about 7.0 to about 8.0; and wherein the ionic component is NaCl, KCl, or LiCl. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label. In embodiments, the composition comprises about 1200 mM TEA and about 850 mM NaCl. In embodiments, the composition comprises about 1600 mM TEA and about 850 mM NaCl. In embodiments, the composition comprises about 1200 mM TEA and about 850 mM KCl. In embodiments, the composition comprises about 1600 mM TEA and about 850 mM KCl. In embodiments, the composition comprises about 1200 mM TEA and about 850 mM LiCl. In embodiments, the composition comprises about 1600 mM TEA and about 850 mM LiCl. In embodiments, the composition has a pH of about 7.5. In embodiments, the composition has a pH of about 7.8.

In embodiments, the disclosure provides a composition comprising about 1000 mM to about 6500 mM TEA, about 500 mM to about 1500 mM ionic component, and about 0.1 mM to about 10 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1100 mM to about 3500 mM TEA, about 600 mM to about 1200 mM ionic component, and about 0.5 mM to about 8 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM to about 1600 mM TEA, about 700 mM to about 900 mM ionic component, and about 1 mM to about 5 mM surfactant, wherein the composition has a pH of about 7.4 to about 7.9; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 850 mM ionic component, and about 1 mM surfactant, wherein the composition has a pH of about 7.4 to about 7.9; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO 18 (PLURONIC® P-123), PEG₅-PPG₆₈-PEG₅(PLURONIC® L-121), PPO₂₆-PEO₅-PPO 26 (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1600 mM TEA, about 850 mM ionic component, and about 1 mM surfactant, wherein the composition has a pH of about 7.4 to about 7.9; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 3200 mM TEA, about 850 mM ionic component, and about 1 mM surfactant, wherein the composition has a pH of about 7.4 to about 7.9; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 6400 mM TEA, about 850 mM ionic component, and about 1 mM surfactant, wherein the composition has a pH of about 7.4 to about 7.9; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 700 mM to about 900 mM ionic component, and about 1 mM to about 5 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-72-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1600 mM TEA, about 700 mM to about 900 mM ionic component, and about 1 mM to about 5 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 3200 mM TEA, about 700 mM to about 900 mM ionic component, and about 1 mM to about 5 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 6400 mM TEA, about 700 mM to about 900 mM ionic component, and about 1 mM to about 5 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1000 mM to about 6500 mM TEA, about 850 mM NaCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1000 mM to about 6500 mM TEA, about 850 mM KCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1000 mM to about 6500 mM TEA, about 850 mM LiCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1000 mM to about 6500 mM TEA, about 700 mM to about 900 mM ionic component, and about 1 mM surfactant, wherein the composition has a pH of about 7.0 to about 8.0; wherein the surfactant is 2-[4-(2,4,4-trimethylpentan-2-yl)phenoxy]ethanol (TRITON™ X-100). In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1000 mM to about 6500 mM TEA, about 700 mM to about 900 mM ionic component, and about 1 mM surfactant, wherein the composition has a pH of about 7.0 to about 8.0; wherein the ionic component is NaCl, KCl, or LiCl; and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1000 mM to about 6500 mM TEA, about 700 mM to about 900 mM ionic component, and about 1 mM surfactant, wherein the composition has a pH of about 7.0 to about 8.0; wherein the ionic component is NaCl, KCl, or LiCl; and wherein the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1000 mM to about 6500 mM TEA, about 700 mM to about 900 mM ionic component, and about 1 mM to about 5 mM surfactant, wherein the composition has a pH of about 7.5; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1000 mM to about 6500 mM TEA, about 700 mM to about 900 mM ionic component, and about 1 mM to about 5 mM surfactant, wherein the composition has a pH of about 7.8; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 850 mM NaCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 1200 mM TEA, about 850 mM NaCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 1600 mM TEA, about 850 mM NaCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 1600 mM TEA, about 850 mM NaCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 3200 mM TEA, about 850 mM NaCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 3200 mM TEA, about 850 mM NaCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 6400 mM TEA, about 850 mM NaCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 6400 mM TEA, about 850 mM NaCl, and about 1 mM 2-[4-(2,4,4-trimethylpentan-2-yl)phenoxy]ethanol (TRITON™ X-100), wherein the composition has a pH of about 7.8. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 850 mM KCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 1200 mM TEA, about 850 mM KCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 1600 mM TEA, about 850 mM KCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 1600 mM TEA, about 850 mM KCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 3200 mM TEA, about 850 mM KCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 3200 mM TEA, about 850 mM KCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 6400 mM TEA, about 850 mM KCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 6400 mM TEA, about 850 mM KCl, and about 1 mM 2-[4-(2,4,4-trimethylpentan-2-yl)phenoxy]ethanol (TRITON™ X-100), wherein the composition has a pH of about 7.8. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 850 mM LiCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 1200 mM TEA, about 850 mM LiCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 1600 mM TEA, about 850 mM LiCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 1600 mM TEA, about 850 mM LiCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 3200 mM TEA, about 850 mM LiCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 3200 mM TEA, about 850 mM LiCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 6400 mM TEA, about 850 mM LiCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 6400 mM TEA, about 850 mM LiCl, and about 1 mM 2-[4-(2,4,4-trimethylpentan-2-yl)phenoxy]ethanol (TRITON™ X-100), wherein the composition has a pH of about 7.8. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 850 mM NaCl, and about 1 mM surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 1200 mM TEA, about 850 mM NaCl, and about 1 mM surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1600 mM TEA, about 850 mM NaCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 1600 mM TEA, about 850 mM NaCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 3200 mM TEA, about 850 mM NaCl, and about 1 mM surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 3200 mM TEA, about 850 mM NaCl, and about 1 mM surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 6400 mM TEA, about 850 mM NaCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 6400 mM TEA, about 850 mM NaCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 850 mM KCl, and about 1 mM surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 1200 mM TEA, about 850 mM KCl, and about 1 mM surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis (propoxylate-block-ethoxyl ate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1600 mM TEA, about 850 mM KCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 1600 mM TEA, about 850 mM KCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 3200 mM TEA, about 850 mM KCl, and about 1 mM surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 3200 mM TEA, about 850 mM KCl, and about 1 mM surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 6400 mM TEA, about 850 mM KCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 6400 mM TEA, about 850 mM KCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 850 mM LiCl, and about 1 mM surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 1200 mM TEA, about 850 mM LiCl, and about 1 mM surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1600 mM TEA, about 850 mM LiCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 1600 mM TEA, about 850 mM LiCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 3200 mM TEA, about 850 mM LiCl, and about 1 mM surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 3200 mM TEA, about 850 mM LiCl, and about 1 mM surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 6400 mM TEA, about 850 mM LiCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 6400 mM TEA, about 850 mM LiCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 850 mM NaCl, and about 1 mM PEG(18) tridecyl ether, wherein the composition has a pH of about 7.5. In embodiments, the disclosure provides a composition comprising about 1200 mM TEA, about 850 mM NaCl, and about 1 mM PEG(18) tridecyl ether, wherein the composition has a pH of about 7.8. In embodiments, the disclosure provides a composition comprising about 1600 mM TEA, about 850 mM NaCl, and about 1 mM PEG(18) tridecyl ether, wherein the composition has a pH of about 7.5. In embodiments, the disclosure provides a composition comprising about 1600 mM TEA, about 850 mM NaCl, and about 1 mM PEG(18) tridecyl ether, wherein the composition has a pH of about 7.8. In embodiments, the disclosure provides a composition comprising about 3200 mM TEA, about 850 mM NaCl, and about 1 mM PEG(18) tridecyl ether, wherein the composition has a pH of about 7.5. In embodiments, the disclosure provides a composition comprising about 3200 mM TEA, about 850 mM NaCl, and about 1 mM PEG(18) tridecyl ether, wherein the composition has a pH of about 7.8. In embodiments, the disclosure provides a composition comprising about 6400 mM TEA, about 850 mM NaCl, and about 1 mM PEG(18) tridecyl ether, wherein the composition has a pH of about 7.5. In embodiments, the disclosure provides a composition comprising about 6400 mM TEA, about 850 mM NaCl, and about 1 mM PEG(18) tridecyl ether, wherein the composition has a pH of about 7.8. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 850 mM KCl, and about 1 mM PEG(18) tridecyl ether, wherein the composition has a pH of about 7.5. In embodiments, the disclosure provides a composition comprising about 1200 mM TEA, about 850 mM KCl, and about 1 mM PEG(18) tridecyl ether, wherein the composition has a pH of about 7.8. In embodiments, the disclosure provides a composition comprising about 1600 mM TEA, about 850 mM KCl, and about 1 mM PEG(18) tridecyl ether, wherein the composition has a pH of about 7.5. In embodiments, the disclosure provides a composition comprising about 1600 mM TEA, about 850 mM KCl, and about 1 mM PEG(18) tridecyl ether, wherein the composition has a pH of about 7.8. In embodiments, the disclosure provides a composition comprising about 3200 mM TEA, about 850 mM KCl, and about 1 mM PEG(18) tridecyl ether, wherein the composition has a pH of about 7.5. In embodiments, the disclosure provides a composition comprising about 3200 mM TEA, about 850 mM KCl, and about 1 mM PEG(18) tridecyl ether, wherein the composition has a pH of about 7.8. In embodiments, the disclosure provides a composition comprising about 6400 mM TEA, about 850 mM KCl, and about 1 mM PEG(18) tridecyl ether, wherein the composition has a pH of about 7.5. In embodiments, the disclosure provides a composition comprising about 6400 mM TEA, about 850 mM KCl, and about 1 mM PEG(18) tridecyl ether, wherein the composition has a pH of about 7.8. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising about 1200 mM TEA, about 850 mM LiCl, and about 1 mM PEG(18) tridecyl ether, wherein the composition has a pH of about 7.5. In embodiments, the disclosure provides a composition comprising about 1200 mM TEA, about 850 mM LiCl, and about 1 mM PEG(18) tridecyl ether, wherein the composition has a pH of about 7.8. In embodiments, the disclosure provides a composition comprising about 1600 mM TEA, about 850 mM LiCl, and about 1 mM PEG(18) tridecyl ether, wherein the composition has a pH of about 7.5. In embodiments, the disclosure provides a composition comprising about 1600 mM TEA, about 850 mM LiCl, and about 1 mM PEG(18) tridecyl ether, wherein the composition has a pH of about 7.8. In embodiments, the disclosure provides a composition comprising about 3200 mM TEA, about 850 mM LiCl, and about 1 mM PEG(18) tridecyl ether, wherein the composition has a pH of about 7.5. In embodiments, the disclosure provides a composition comprising about 3200 mM TEA, about 850 mM LiCl, and about 1 mM PEG(18) tridecyl ether, wherein the composition has a pH of about 7.8. In embodiments, the disclosure provides a composition comprising about 6400 mM TEA, about 850 mM LiCl, and about 1 mM PEG(18) tridecyl ether, wherein the composition has a pH of about 7.5. In embodiments, the disclosure provides a composition comprising about 6400 mM TEA, about 850 mM LiCl, and about 1 mM PEG(18) tridecyl ether, wherein the composition has a pH of about 7.8. In embodiments, the composition is substantially free of an additional pH buffering component. In embodiments, the composition is substantially free of an additional component having a pKa of about 7.0 to about 8.0. In embodiments, the composition does not comprise any of Tris, phosphate, HEPES, glycylglycine, borate, acetate, citrate, POPSO, HEPPSO, and PIPES. In embodiments, the composition further comprises an ECL-labeled component. In embodiments, the ECL-labeled component is a detection reagent comprising an ECL label.

In embodiments, the disclosure provides a composition comprising: TEA, an ionic component, and optionally, one or both of an ECL-labeled component and a surfactant; wherein the composition has a pH of about 7.0 to about 8.0, and optionally, wherein the composition is substantially free of an additional pH buffering component. In embodiments, the composition comprises about 1000 mM to about 6500 mM of the TEA, and about 500 mM to about 2000 mM of the ionic component. In embodiments, the surfactant comprises an alkyl ether-PEG. In embodiments, the composition is substantially free of an additional pH buffering component. In embodiments, the composition consists essentially of or consists of the recited components.

In embodiments, the disclosure provides a composition comprising: TEA, an ionic component, and one or both of an ECL-labeled component and a surfactant; wherein the composition has a pH of about 7.0 to about 8.0, and optionally, wherein the composition is substantially free of an additional pH buffering component. In embodiments, the composition comprises about 1000 mM to about 6500 mM of the TEA, and about 500 mM to about 2000 mM of the ionic component. In embodiments, the surfactant comprises an alkyl ether-PEG. In embodiments, the composition is substantially free of an additional pH buffering component. In embodiments, the composition consists essentially of or consists of the recited components.

In embodiments, the disclosure provides a composition comprising: TEA, an ionic component, and optionally, one or both of an ECL-labeled component and a surfactant; wherein the composition has a pH of about 7.0 to about 8.0, and wherein the composition is substantially free of an additional pH buffering component. In embodiments, the composition comprises about 1000 mM to about 6500 mM of the TEA, and about 500 mM to about 2000 mM of the ionic component. In embodiments, the surfactant comprises an alkyl ether-PEG. In embodiments, the composition consists essentially of or consists of the recited components.

In embodiments, the disclosure provides a composition comprising: TEA, an ionic component, and an ECL-labeled component; wherein the composition has a pH of about 7.0 to about 8.0. In embodiments, the disclosure provides a composition comprising: TEA, an ionic component, and a surfactant; wherein the composition has a pH of about 7.0 to about 8.0. In embodiments, the disclosure provides a composition comprising: TEA, an ionic component, an ECL-labeled component, and a surfactant; wherein the composition has a pH of about 7.0 to about 8.0. In embodiments, the composition comprises about 1000 mM to about 6500 mM of the TEA, and about 500 mM to about 2000 mM of the ionic component. In embodiments, the surfactant comprises an alkyl ether-PEG. In embodiments, the composition is substantially free of an additional pH buffering component. In embodiments, the composition consists essentially of or consists of the recited components.

In embodiments, the composition provided herein is in dry form. In embodiments, the composition provided herein is in the form of a dry powder. In embodiments, the composition provided herein is a lyophilized powder. Throughout the present disclosure, when a composition comprises a certain concentration of its recited components (e.g., about 1000 mM to about 6500 mM of TEA; about 500 mM to about 2000 mM of ionic component; and/or about 0.1 mM to about 10 mM of surfactant) and/or a certain pH of its recited components (e.g., a pH of about 7.0 to about 8.0), it will be understood by one of ordinary skill in the art that the recited concentrations of the components and pH of the composition are in reference to the composition in liquid form, e.g., a dry composition reconstituted with a liquid diluent (e.g., water or aqueous assay buffer). In embodiments, the composition is in dry form and comprises the recited components at the recited concentrations when reconstituted with a liquid diluent. In embodiments, the composition is in dry form and comprises the recited pH when constituted with a liquid diluent.

### Alkyl Diethanolamine/Zwitterionic Tertiary Amine Compositions

In embodiments, the disclosure provides a composition comprising about 50 mM to about 250 mM tBDEA, about 500 mM to about 1500 mM ionic component, and about 0.1 mM to about 10 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 50 mM to about 250 mM MDEA, about 500 mM to about 1500 mM ionic component, and about 0.1 mM to about 10 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 50 mM to about 250 mM DEA-PS, about 500 mM to about 1500 mM ionic component, and about 0.1 mM to about 10 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM tBDEA, about 700 mM to about 900 mM ionic component, and about 1 mM to about 5 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM MDEA, about 700 mM to about 900 mM ionic component, and about 1 mM to about 5 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM DEA-PS, about 700 mM to about 900 mM ionic component, and about 1 mM to about 5 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 850 mM ionic component, and about 1 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM MDEA, about 850 mM ionic component, and about 1 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM DEA-PS, about 850 mM ionic component, and about 1 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 700 mM to about 900 mM ionic component, and about 1 mM to about 5 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM MDEA, about 700 mM to about 900 mM ionic component, and about 1 mM to about 5 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM DEA-PS, about 700 mM to about 900 mM ionic component, and about 1 mM to about 5 mM surfactant, wherein the composition has a pH of about 7.0 to about 8.0; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM tBDEA, about 850 mM NaCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM MDEA, about 850 mM NaCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM DEA-PS, about 850 mM NaCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM tBDEA, about 850 mM KCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM MDEA, about 850 mM KCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM DEA-PS, about 850 mM KCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM tBDEA, about 850 mM LiCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM MDEA, about 850 mM LiCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM DEA-PS, about 850 mM LiCl, and about 1 mM to about 5 mM surfactant, wherein the composition has a pH of about 7.0 to about 8.0; wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM tBDEA, about 700 mM to about 900 mM ionic component, and about 1 mM surfactant, wherein the composition has a pH of about 7.0 to about 8.0; wherein the surfactant is 2-[4-(2,4,4-trimethylpentan-2-yl)phenoxy]ethanol (TRITON™ X-100). In embodiments, the disclosure provides a composition comprising about 100 mM to about 200 mM MDEA, about 700 mM to about 900 mM ionic component, and about 1 mM surfactant, wherein the composition has a pH of about 7.0 to about 8.0; wherein the surfactant is 2-[4-(2,4,4-trimethylpentan-2-yl)phenoxy]ethanol (TRITON™ X-100). In embodiments, the disclosure provides a composition comprising about 100 mM to about 200 mM DEA-PS, about 700 mM to about 900 mM ionic component, and about 1 mM surfactant, wherein the composition has a pH of about 7.0 to about 8.0; wherein the surfactant is 2-[4-(2,4,4-trimethylpentan-2-yl)phenoxy]ethanol (TRITON™ X-100). In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM tBDEA, about 700 mM to about 900 mM ionic component, and about 1 mM surfactant, wherein the composition has a pH of about 7.0 to about 8.0; wherein the ionic component is NaCl, KCl, or LiCl; and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 100 mM to about 200 mM MDEA, about 700 mM to about 900 mM ionic component, and about 1 mM surfactant, wherein the composition has a pH of about 7.0 to about 8.0; wherein the ionic component is NaCl, KCl, or LiCl; and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 100 mM to about 200 mM DEA-PS, about 700 mM to about 900 mM ionic component, and about 1 mM surfactant, wherein the composition has a pH of about 7.0 to about 8.0; wherein the ionic component is NaCl, KCl, or LiCl; and wherein the surfactant is Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM tBDEA, about 700 mM to about 900 mM ionic component, and about 1 mM surfactant, wherein the composition has a pH of about 7.0 to about 8.0; wherein the ionic component is NaCl, KCl, or LiCl; and wherein the surfactant is PEG(18) tridecyl ether. In embodiments, the disclosure provides a composition comprising about 100 mM to about 200 mM MDEA, about 700 mM to about 900 mM ionic component, and about 1 mM surfactant, wherein the composition has a pH of about 7.0 to about 8.0; wherein the ionic component is NaCl, KCl, or LiCl; and wherein the surfactant is PEG(18) tridecyl ether. In embodiments, the disclosure provides a composition comprising about 100 mM to about 200 mM DEA-PS, about 700 mM to about 900 mM ionic component, and about 1 mM surfactant, wherein the composition has a pH of about 7.0 to about 8.0; wherein the ionic component is NaCl, KCl, or LiCl; and wherein the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM tBDEA, about 700 mM to about 900 mM ionic component, and about 1 mM to about 5 mM surfactant, wherein the composition has a pH of about 7.5; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM tBDEA, about 700 mM to about 900 mM ionic component, and about 1 mM to about 5 mM surfactant, wherein the composition has a pH of about 7.8; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM MDEA, about 700 mM to about 900 mM ionic component, and about 1 mM to about 5 mM surfactant, wherein the composition has a pH of about 7.5; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM MDEA, about 700 mM to about 900 mM ionic component, and about 1 mM to about 5 mM surfactant, wherein the composition has a pH of about 7.8; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM DEA-PS, about 700 mM to about 900 mM ionic component, and about 1 mM to about 5 mM surfactant, wherein the composition has a pH of about 7.5; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 100 mM to about 200 mM DEA-PS, about 700 mM to about 900 mM ionic component, and about 1 mM to about 5 mM surfactant, wherein the composition has a pH of about 7.8; wherein the ionic component is NaCl, KCl, or LiCl; and wherein the surfactant is 2-[4-(2,4,4-trimethylpentan-2-yl)phenoxy]ethanol (TRITON™ X-100), Poloxamer 407 (KOLLIPHOR® P-407), PEO₁₈-PPO-₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the surfactant is PEG(18) tridecyl ether. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 850 mM NaCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 150 mM tBDEA, about 850 mM NaCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 150 mM MDEA, about 850 mM NaCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 150 mM MDEA, about 850 mM NaCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 150 mM DEA-PS, about 850 mM NaCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 150 mM DEA-PS, about 850 mM NaCl, and about 1 mM 2-[4-(2,4,4-trimethylpentan-2-yl)phenoxy]ethanol (TRITON™ X-100), wherein the composition has a pH of about 7.8. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 850 mM KCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 150 mM tBDEA, about 850 mM KCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 150 mM MDEA, about 850 mM KCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 150 mM MDEA, about 850 mM KCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 150 mM DEA-PS, about 850 mM KCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 150 mM DEA-PS, about 850 mM KCl, and about 1 mM 2-[4-(2,4,4-trimethylpentan-2-yl)phenoxy]ethanol (TRITON™ X-100), wherein the composition has a pH of about 7.8. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 850 mM LiCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 150 mM tBDEA, about 850 mM LiCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 150 mM MDEA, about 850 mM LiCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 150 mM MDEA, about 850 mM LiCl, and about 1 mM 2-[4-(2,4,4-trimethylpentan-2-yl)phenoxy]ethanol (TRITON™ X-100), wherein the composition has a pH of about 7.8. In embodiments, the disclosure provides a composition comprising about 150 mM DEA-PS, about 850 mM LiCl, and about 1 mM 2-[4-(2,4,4-trimethylpentan-2-yl)phenoxy]ethanol (TRITON™ X-100), wherein the composition has a pH of about 7.5. In embodiments, the disclosure provides a composition comprising about 150 mM DEA-PS, about 850 mM LiCl, and about 1 mM 2-[4-(2,4,4-trimethylpentan-2-yl)phenoxy]ethanol (TRITON™ X-100), wherein the composition has a pH of about 7.8. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 850 mM NaCl, and about 1 mM surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 150 mM tBDEA, about 850 mM NaCl, and about 1 mM surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM MDEA, about 850 mM NaCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 150 mM MDEA, about 850 mM NaCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM DEA-PS, about 850 mM NaCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 150 mM DEA-PS, about 850 mM NaCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 850 mM KCl, and about 1 mM surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 150 mM tBDEA, about 850 mM KCl, and about 1 mM surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC® 31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM MDEA, about 850 mM KCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 150 mM MDEA, about 850 mM KCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM DEA-PS, about 850 mM KCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 150 mM DEA-PS, about 850 mM KCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 850 mM LiCl, and about 1 mM surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 150 mM tBDEA, about 850 mM LiCl, and about 1 mM surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM MDEA, about 850 mM LiCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 150 mM MDEA, about 850 mM LiCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM DEA-PS, about 850 mM LiCl, and about surfactant, wherein the composition has a pH of about 7.5, and wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the disclosure provides a composition comprising about 150 mM DEA-PS, about 850 mM LiCl, and about 1 surfactant, wherein the composition has a pH of about 7.8, wherein the surfactant is Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, an alkyl ether-polyethylene glycol (PEG), or a combination thereof. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 850 mM NaCl, and about 1 mM PEG(18) tridecyl ether, wherein the composition has a pH of about 7.5. In embodiments, the disclosure provides a composition comprising about 150 mM tBDEA, about 850 mM NaCl, and about 1 mM PEG(18) tridecyl ether, wherein the composition has a pH of about 7.8. In embodiments, the disclosure provides a composition comprising about 150 mM MDEA, about 850 mM NaCl, and about 1 mM PEG(18) tridecyl ether, wherein the composition has a pH of about 7.5. In embodiments, the disclosure provides a composition comprising about 150 mM MDEA, about 850 mM NaCl, and about 1 mM PEG(18) tridecyl ether, wherein the composition has a pH of about 7.8. In embodiments, the disclosure provides a composition comprising about 150 mM DEA-PS, about 850 mM NaCl, and about 1 mM PEG(18) tridecyl ether, wherein the composition has a pH of about 7.5. In embodiments, the disclosure provides a composition comprising about 150 mM DEA-PS, about 850 mM NaCl, and about 1 mM PEG(18) tridecyl ether, wherein the composition has a pH of about 7.8. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 850 mM KCl, and about 1 mM PEG(18) tridecyl ether, wherein the composition has a pH of about 7.5. In embodiments, the disclosure provides a composition comprising about 150 mM tBDEA, about 850 mM KCl, and about 1 mM PEG(18) tridecyl ether, wherein the composition has a pH of about 7.8. In embodiments, the disclosure provides a composition comprising about 150 mM MDEA, about 850 mM KCl, and about 1 mM PEG(18) tridecyl ether, wherein the composition has a pH of about 7.5. In embodiments, the disclosure provides a composition comprising about 150 mM MDEA, about 850 mM KCl, and about 1 mM PEG(18) tridecyl ether, wherein the composition has a pH of about 7.8. In embodiments, the disclosure provides a composition comprising about 150 mM DEA-PS, about 850 mM KCl, and about 1 mM PEG(18) tridecyl ether, wherein the composition has a pH of about 7.5. In embodiments, the disclosure provides a composition comprising about 150 mM DEA-PS, about 850 mM KCl, and about 1 mM PEG(18) tridecyl ether, wherein the composition has a pH of about 7.8. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising about 150 mM tBDEA, about 850 mM LiCl, and about 1 mM PEG(18) tridecyl ether, wherein the composition has a pH of about 7.5. In embodiments, the disclosure provides a composition comprising about 150 mM tBDEA, about 850 mM LiCl, and about 1 mM PEG(18) tridecyl ether, wherein the composition has a pH of about 7.8. In embodiments, the disclosure provides a composition comprising about 150 mM MDEA, about 850 mM LiCl, and about 1 mM PEG(18) tridecyl ether, wherein the composition has a pH of about 7.5. In embodiments, the disclosure provides a composition comprising about 150 mM MDEA, about 850 mM LiCl, and about 1 mM PEG(18) tridecyl ether, wherein the composition has a pH of about 7.8. In embodiments, the disclosure provides a composition comprising about 150 mM DEA-PS, about 850 mM LiCl, and about 1 mM PEG(18) tridecyl ether, wherein the composition has a pH of about 7.5. In embodiments, the disclosure provides a composition comprising about 150 mM DEA-PS, about 850 mM LiCl, and about 1 mM PEG(18) tridecyl ether, wherein the composition has a pH of about 7.8. In embodiments, the composition further comprises about 100 mM to about 200 mM pH buffering component. In embodiments, the pH buffering component is Tris, phosphate, HEPES, glycylglycine, borate, acetate, citrate, or a combination thereof. In embodiments, the composition further comprises an ECL-labeled component. In embodiments, the ECL-labeled component is a detection reagent comprising an ECL label.

In embodiments, the disclosure provides a composition comprising an ECL coreactant, an ionic component, and a surfactant. In embodiments, the ECL coreactant is tributylamine (TBA), (dibutyl)aminoethanol (DBAE), (diethyl)aminoethanol (DEAE), triethanolamine (TEA), butyldiethanolamine (BDEA), propyldiethanolamine (PDEA), ethyldiethanolamine (EDEA), methyldiethanolamine (MDEA), tert-butyldiethanolamine (tBDEA), dibutylamine (DBA), butylethanolamine (BEA), diethanolamine (DEA), dibutylamine propylsulfonate (DBA-PS), dibutylamine butylsulfonate (DBA-BS), butylethanolamine propylsulfonate (BEA-PS), butylethanolamine butylsulfonate (BEA-BS), diethanolamine propylsulfonate (also known as 3-[Bis-(2-hydroxy-ethyl)-amino]-propane-1-sulfonic acid; DEA-PS), diethanolamine butylsulfonate (DEA-BS), or a combination thereof. In embodiments, the composition has a pH of about 7.0 to about 8.0. In embodiments, the composition further comprises a pH buffering component. Suitable ionic components (e.g., NaCl, KCl, and LiCl), surfactants (e.g., TRITON X-100 or mild surfactants described herein), pH buffering components (e.g., Tris or phosphate), and their concentrations in the compositions are provided herein. In embodiments, the composition further comprises an ECL-labeled component. In embodiments, the ECL-labeled component is a detection reagent comprising an ECL label.

### Methods

In embodiments, the disclosure provides a method of generating electrochemiluminescence (ECL), comprising: (a) contacting an electrode with an ECL coreactant composition provided herein, (b) applying a voltage to the electrode; and (c) generating ECL. In embodiments, the ECL coreactant composition comprises TEA, tBDEA, BDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the disclosure provides a method of generating electrochemiluminescence (ECL), comprising: (a) contacting an electrode with a TEA composition comprising TEA; an ionic component; and optionally a surfactant; (b) applying a voltage to the electrode; and (c) generating ECL. In embodiments, the method further comprises detecting the generated ECL. In embodiments, the method further comprises measuring the generated ECL. In embodiments, the electrode is present on a surface.

In embodiments, the disclosure provides a method of generating electrochemiluminescence (ECL), comprising: (a) contacting an electrode with: (i) an ECL coreactant composition provided herein and (ii) an ECL label; (b) applying a voltage to the electrode; and (c) generating ECL. In embodiments, the ECL coreactant composition comprises TEA, tBDEA, BDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the disclosure provides a method of generating electrochemiluminescence (ECL), comprising: (a) contacting an electrode with: (i) a TEA composition comprising TEA, an ionic component, and optionally a surfactant; and (ii) an ECL label; (b) applying a voltage to the electrode; and (c) generating ECL. In embodiments, the method further comprises detecting the generated ECL. In embodiments, the method further comprises measuring the generated ECL, thereby quantifying the amount of the ECL label. In embodiments, the electrode is present on a surface.

In embodiments, the disclosure provides a method of quantifying the amount of an ECL label in a sample, comprising: (a) contacting an electrode with (i) an ECL coreactant composition provided herein or a TEA composition provided herein; and (ii) the sample comprising the ECL label; (b) applying a voltage to the electrode; (c) generating ECL; (d) measuring the ECL; and (e) quantifying the amount of the ECL label from the measured ECL. In embodiments, the ECL coreactant composition comprises TEA, tBDEA, BDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the TEA composition comprises TEA, an ionic component, and optionally a surfactant.

In embodiments, the ECL is generated from the reaction between the ECL coreactant (e.g., TEA, tBDEA, MDEA, and/or DEA-PS) in the compositions herein and the ECL label. In embodiments, the ECL label is present on an ECL-labeled component. In embodiments, the ECL label is present in a sample. In embodiments, the sample comprises the ECL-labeled component. In embodiments, the ECL-labeled component comprises a detection reagent. In embodiments, the sample comprises a binding partner of the ECL-labeled component. In embodiments, the ECL-labeled component comprises a binding partner of a detection reagent. In embodiments, the detection reagent is part of a binding complex that comprises the detection reagent, an analyte, and a capture reagent wherein the capture reagent comprises a binding partner to a binding reagent that is immbolized on a surface. In embodiments, the ECL-labeled component is an oligonucleotide probe that is complementary to an extended primer that is attached to a detection reagent (e.g., a detection antibody) that binds to a target analyte such as a peptide or protein. Detection reagents and binding partners thereof are further described herein. In embodiments, the ECL-labeled component is present in a binding complex, and the method further comprises detecting the binding complex by detecting the generated ECL. In embodiments, the method comprises contacting the electrode with the sample that comprises a binding partner of the ECL-labeled component, wherein the ECL-labeled component and the binding partner form a binding complex, and the method further comprises detecting the binding complex by detecting the generated ECL. In embodiments, the method comprises measuring the generated ECL, thereby quantifying the amount of the ECL-labeled component and/or the binding complex.

In embodiments, each of the sample, the ECL coreactant composition or the TEA composition provided herein, and the ECL-labeled component is dry. In embodiments, In embodiments, each of the sample, the ECL coreactant composition or the TEA composition provided herein, and the ECL-labeled component is liquid. In embodiments, one or more of the sample, the ECL coreactant composition or the TEA composition provided herein, and the ECL-labeled component is dry, and the remaining component(s) is liquid. For example, the sample is liquid, and one or both of the ECL coreactant composition or the TEA composition provided herein and the ECL-labeled component are dry. In embodiments comprising a liquid component and a dry component, the liquid component reconstitutes the dry component. In embodiments, the method further comprises contacting the electrode with a liquid diluent, thereby reconstituting the dried component(s) in the liquid. In embodiments, the dried component(s) are present on the surface. In embodiments, the ECL coreactant composition is dry and present on the surface. In embodiments, the TEA composition is dry and present on the surface. In embodiments, the ECL-labeled component is dry and present on the surface. Compositions in dry form are described herein. In embodiments, the ECL-labeled component is a detection reagent that comprises an ECL label. In embodiments, the ECL coreactant composition comprises TEA, tBDEA, BDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the TEA composition comprises TEA, an ionic component, and optionally a surfactant.

In embodiments, the ECL-labeled component in the binding complex is a first copy of a detection reagent comprising the ECL label. In embodiments, the binding complex comprises the first copy of the detection reagent and a binding reagent immobilized on the surface. Binding reagents are further described herein. In embodiments, the method further comprises forming the binding complex. In embodiments, the binding complex is formed prior to or during step (a) of the method.

In embodiments, the binding complex is formed by incubating an assay mixture comprising the binding reagent, the first copy of the detection reagent, and a second copy of the detection reagent that comprises an ECL label, under conditions wherein the binding complex is formed on the surface, and the second copy of the detection reagent remains in solution. In embodiments, the ECL coreactant composition comprises TEA, BDEA, tBDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In composition comprises TEA, an ionic component, and optionally a surfactant.

In embodiments, the binding complex is formed by incubating an assay mixture comprising the binding reagent, the first copy of the detection reagent, a second copy of the detection reagent that comprises an ECL label, and an ECL coreactant composition or a TEA composition provided herein, under conditions wherein the binding complex is formed on the surface, and the second copy of the detection reagent remains in solution. In embodiments, the ECL coreactant composition comprises TEA, tBDEA, BDEA, MDEA, DEA-PS, or combination thereof. In embodiments, the composition comprises TEA. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the TEA composition comprises TEA, an ionic component, and optionally a surfactant.

In embodiments, the binding complex is formed by combining a sample with the first copy of the detection reagent, a second copy of the detection reagent that comprises an ECL label, and an ECL coreactant composition or a TEA composition provided herein, thereby forming an assay mixture; and contacting the assay mixture with the binding reagent, under conditions wherein the binding complex is formed on the surface, and the second detection reagent remains in solution. In embodiments, the ECL coreactant composition comprises TEA, BDEA, tBDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the composition comprises TEA. In embodiments, the TEA composition comprises TEA, an ionic component, and optionally a surfactant.

In embodiments, the binding complex further comprises an analyte. Analytes are further described herein. In embodiments, the binding reagent and the detection reagent each specifically binds to the analyte. In embodiments, the method comprises detecting the analyte by detecting the generated ECL. In embodiments, the method comprises measuring the generated ECL, thereby quantifying the amount of the analyte.

In embodiments, the ECL label comprises an electrochemiluminescent organometallic complex. In embodiments, the electrochemiluminescent organometallic complex comprises ruthenium, osmium, iridium, rhenium, and/or a lanthanide metal. In embodiments, the ECL label comprises ruthenium. In embodiments, the ECL label comprises ruthenium (II) tris-bipyridine. In embodiments, the electrochemiluminescent organometallic complex comprises a substituted or unsubstituted bipyridine or a substituted or unsubstituted phenanthroline. In embodiments, the ECL label comprises a substituted bipyridine. In embodiments, the ECL label comprises an organometallic complex comprising at least one substituted bipyridine ligand, wherein the substituted bipyridine ligand comprises at least one sulfonate group. In embodiments, the ECL label comprises an organometallic complex comprising at least two substituted bipyridine ligands, wherein each substituted bipyridine ligand comprises at least one sulfonate group. In embodiments, the substituted bipyridine ligand comprising at least one sulfonate group is a compound of Formula I. In embodiments, the ECL label comprises a compound of Formula II.

In embodiments, the compositions herein are used in ECL-based binding assays, e.g., to detect and/or quantify an analyte of interest and/or a binding complex comprising an analyte. In embodiments, a binding complex is formed, e.g., on a surface comprising an electrode, and the binding complex comprises an ECL label capable of generating ECL when contacted with an ECL coreactant described herein. Binding assay formats include, but are not limited to: (1) direct binding assays, in which the analyte of interest is labeled with an ECL label, and a binding reagent, which is a binding partner of the analyte, is immobilized to the surface, and a binding complex is formed by direct binding of the binding reagent and the labeled analyte; (2) sandwich binding assays, in which an immobilized binding reagent and a detection reagent comprising an ECL label are both binding partners of the analyte, and the analyte binds the two binding partners to form the binding complex; (3) competitive binding assays, in which an immobilized binding reagent is a binding partner of the analyte, and a labeled detection reagent is a competitor (e.g., the analyte or a structural analogue of the analyte) that competes with the immobilized binding reagent for binding to the analyte, or, alternatively, the labeled detection reagent is a binding partner of the analyte, and the immobilized binding reagent is a competitor that competes with the detection reagent for binding to the analyte. In competitive binding assays, the labeled binding complex, formed by direct binding of the immobilized binding reagent and labeled detection reagent, decreases in quantity with increasing quantity of analyte. Binding assays are further described, e.g., in WO 2014/165061; WO 2014/160192; WO 2015/175856; U.S. Pat. Nos. 9,618,510; 10,114,015; 10,408,823; US 2017/0168047; and US 2019/0011441, each of which are herein incorporated by reference in its entirety.

In embodiments, the disclosure provides a method for detecting a binding complex, comprising: (a) contacting a liquid sample with a surface comprising an ECL coreactant composition or a TEA composition provided herein, wherein the liquid sample comprises an ECL-labeled component; or wherein the liquid sample comprises a binding partner of an ECL-labeled component, and the method further comprises contacting the surface with the ECL-labeled component, thereby forming a binding complex on the surface that comprises the ECL-labeled component; (b) applying a voltage to the surface to generate ECL; and (c) detecting the generated ECL, thereby detecting the binding complex. In embodiments, the ECL coreactant composition comprises TEA, tBDEA, BDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the ECL coreactant composition comprises TEA. In embodiments, the TEA composition comprises TEA, an ionic component, and optionally a surfactant. In embodiments, the surface comprises an electrode. In embodiments, the ECL-labeled component comprises a detection reagent that comprises an ECL label. In embodiments, the ECL-labeled component comprises a detection reagent that comprises an ECL label, and the binding complex comprises a binding reagent and the detection reagent. In embodiments, the ECL-labeled component comprises a binding partner of a detection reagent, wherein the binding partner comprises an ECL label. In embodiments, the ECL-labeled component comprises a binding partner of a detection reagent, and the binding complex comprises a binding reagent, the detection reagent, and the binding partner. Detection reagents and binding partners are further described herein. In embodiments, the detection reagent and the ECL-labeled component comprise complementary oligonucleotides. In embodiments, the binding complex further comprises an analyte. In embodiments, the binding reagent and the detection reagent each specifically binds to the analyte.

In embodiments, the disclosure provides a method for detecting a binding complex, comprising: (a) contacting a liquid sample with a surface comprising an ECL-labeled component and an ECL coreactant composition or a TEA composition provided herein, wherein the liquid sample comprises a binding partner of an ECL-labeled component, thereby forming a binding complex on the surface that comprises the ECL-labeled component; (b) applying a voltage to the surface to generate ECL; and (c) detecting the generated ECL, thereby detecting the binding complex. In embodiments, the ECL coreactant composition comprises TEA, tBDEA, BDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the ECL coreactant composition comprises TEA. In embodiments, the TEA composition comprises TEA, an ionic component, and optionally a surfactant. In embodiments, the surface comprises an electrode. In embodiments, the ECL-labeled component comprises a detection reagent that comprises an ECL label. In embodiments, the ECL-labeled component comprises a detection reagent that comprises an ECL label, and the binding complex comprises a binding reagent and the detection reagent. In embodiments, the ECL-labeled component comprises a binding partner of a detection reagent, wherein the binding partner comprises an ECL label. In embodiments, the ECL-labeled component comprises a binding partner of a detection reagent, and the binding complex comprises a binding reagent, the detection reagent, and the binding partner. Detection reagents and binding partners are further described herein. In embodiments, the detection reagent and the ECL-labeled component comprise complementary oligonucleotides. In embodiments, the binding complex further comprises an analyte. In embodiments, the binding reagent and the detection reagent each specifically binds to the analyte.

In embodiments, the disclosure provides a method for detecting a binding complex, comprising: (a) forming a binding complex on a surface, and wherein the binding complex comprises an ECL-labeled component; (b) contacting the binding complex with an ECL coreactant composition or a TEA composition provided herein; (c) applying a voltage to the surface to generate ECL; and (d) detecting the generated ECL, thereby detecting the binding complex. In embodiments, the ECL coreactant composition comprises TEA, BDEA, tBDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the ECL coreactant composition comprises TEA. In embodiments, the TEA composition comprises TEA, an ionic component, and optionally a surfactant. In embodiments, the surface comprises an electrode. In embodiments, the ECL-labeled component comprises a detection reagent that comprises an ECL label. In embodiments, the ECL-labeled component comprises a detection reagent that comprises an ECL label, and the binding complex comprises a binding reagent and the detection reagent. In embodiments, the ECL-labeled component comprises a binding partner of a detection reagent, wherein the binding partner comprises an ECL label. In embodiments, the ECL-labeled component comprises a binding partner of a detection reagent, and the binding complex comprises a binding reagent, the detection reagent, and the binding partner. Detection reagents and binding partners are further described herein. In embodiments, the detection reagent and the ECL-labeled component comprise complementary oligonucleotides. In embodiments, the binding complex further comprises an analyte. In embodiments, the binding reagent and the detection reagent each specifically binds to the analyte.

In embodiments, the disclosure provides a method for detecting a binding complex, comprising: (a) forming a binding complex on a surface, wherein the surface comprises an electrode, and wherein the binding complex comprises a binding reagent immobilized on the surface and a detection reagent comprising an electrochemiluminescence (ECL) label; (b) contacting the binding complex with an ECL coreactant composition or a TEA composition provided herein; (c) applying a voltage to the surface to generate ECL; and (d) detecting the generated ECL, thereby detecting the binding complex. In embodiments, the binding complex further comprises an analyte, and the binding reagent and the detection reagent each specifically binds to the analyte. In embodiments, the ECL coreactant composition comprises TEA, BDEA, tBDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the ECL coreactant composition comprises TEA. In embodiments, the TEA composition comprises TEA, an ionic component, and optionally a surfactant. In embodiments, the binding complex further comprises an analyte. In embodiments, the binding reagent and the detection reagent each specifically binds to the analyte.

In embodiments, the disclosure provides a method for detecting an analyte of interest in a sample, comprising: (a) contacting the sample with: (i) a surface comprising a binding reagent, wherein the binding reagent specifically binds to the analyte; and (ii) a detection reagent that specifically binds to the analyte, wherein the detection reagent comprises an electrochemiluminescence (ECL) label, thereby forming a binding complex on the surface comprising the binding reagent, the analyte, and the detection reagent; (b) contacting the binding complex on the surface with an ECL coreactant composition or a TEA composition provided herein; (c) applying a voltage to the surface to generate ECL; and (d) detecting the generated ECL, thereby detecting the analyte. In embodiments, the ECL coreactant composition comprises TEA, BDEA, tBDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the ECL coreactant composition comprises TEA. In embodiments, the TEA composition comprises TEA, an ionic component, and optionally a surfactant. In embodiments, the surface comprises an electrode. In embodiments, the analyte of interest is an oligonucleotide, and the detection reagent that specifically binds to the analyte is an oligonucleotide probe that comprises an ECL label and a quenching moiety which quenches the ECL signal at least when the ECL-labeled oligonucleotide probe is not hybridized to a complementary oligonucleotide. In emobdiments, the ECL-labeled oligonucleotide probe comprises a stem-loop or hairpin structure, an ECL label, and a quenching moiety, wherein said quenching moiety is in proximity to the ECL label and quenches the ECL label when the ECL-labeled oligonucleotide probe is in a stem-loop or hairpin configuration, but does not quench the ECL label when the stem-loop or hairpin structure is in an open configuration (e.g., as illustrated in FIGS. 14 and 20). In embodiments, the ECL-labeled oligonucleotide probe with the stem-loop or hairpin structure is an ECL-labeled molecular beacon probe. In embodiments, the ECL-labeled oligonucleotide probes comprise an ECL label and a quencher that are sufficiently close to each other on the probe when the probe is in a linear configuration (e.g., not in a stem-loop or hairpin configuration) that the quenching moiety quenches the ECL signal whether the probe is hybridized to a complementary oligonucleotide or not (e.g., as illustrated in FIG. 21).

In embodiments, the disclosure provides a method for detecting an analyte of interest in a sample, comprising contacting the sample with: (i) a surface comprising a binding reagent, wherein the binding reagent specifically binds to a binding complex comprising the analyte (e.g., a peptide); (ii), a capture reagent (e.g., an antibody) that specifically binds to the analyte, wherein the capture reagent additionally comprises a binding partner to the binding reagent (e.g., biotin/streptavidin); (iii) a detection reagent that specifically binds to the analyte, wherein the detection reagent comprises an oligonucleotide primer (e.g., oligonucleotide lableled antibody); (iv) a template oligonucleotide; and (v) ECL-labeled oligonucleotide probe. In embodiments, the capture reagent and the detection reagent bind to the analyte, the primer binds to the template oligonucleotide, the primer is extended via amplification of the template oligonucleotide, and the ECL-labeled oligonucleotide probe binds to the extended primer oligonucleotide (e.g., as illustrated in FIG. 20). In embodiments, the binding complex comprises the capture reagent comprising the binding partner, the analyte, and the detection reagent comprising the extended oligonucleotide primer. The surface is contacted with an ECL coreactant composition or a TEA composition provided herein, a voltage is applied to the surface to generate ECL, and the generated ECL is detected, thereby detecting the analyte. In embodiments, the ECL coreactant composition comprises TEA, BDEA, tBDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the ECL coreactant composition comprises TEA. In embodiments, the TEA composition comprises TEA, an ionic component, and optionally a surfactant. In embodiments, the surface comprises an electrode. In embodiments, the ECL-labeled oligonucleotide probe is a molecular beacon probe.

As discussed herein, the compositions provided herein can be used in an ECL-based assay that does not require a wash step. A “wash step,” as used in the context of ECL-based assays conducted on a surface, refers to adding a wash buffer to the surface to remove undesired components from the assay reaction mixture, e.g., excess, non-specifically bound, or unbound reagents (e.g., detection reagent and/or ECL label) and/or unbound or non-specifically bound components of the sample. In an example, a biological sample can contain an analyte of interest and various other biological materials that are not of interest and do not bind specifically to the binding reagent, and a wash step can remove such components from the reaction mixture. In embodiments, the composition comprises TEA. In embodiments, the composition comprises TEA, an ionic component, and optionally a surfactant. In a “washed” assay, a wash step is typically used to remove unbound ECL labels prior to detecting the ECL labels on the surface. The wash step may be eliminated if the detection method can effectively discriminate between an ECL label bound to the surface (e.g., as part of a binding complex to be detected) or an unbound, “free” ECL label in solution. A “non-wash” assay format, which eliminates the wash step, is often advantageous because the washing step can be difficult or cumbersome to perform in many circumstances. However, a non-wash assay format is typically difficult to develop due to high background ECL signal from incomplete discrimination of free vs. bound ECL labels present in the reaction mixture. Even in assays employing a wash step, good discrimination between bound and free ECL label is advantageous because it provides greater robustness to inefficiencies or variations in the quality of a wash by providing tolerance to low levels of free label contamination that might be associated with a poor quality wash.

As discussed herein, the compositions herein surprisingly discriminated free vs. bound ECL labels in ECL-based assays conducted on solid surfaces (e.g., a solid electrode surface). In embodiments, the compositions herein increase the ratio of ECL signal from bound label to ECL signal from free label. Thus, the compositions herein provided improved assay performance, particularly when measuring low affinity interactions, which require the presence of the ECL label in high concentrations in the reaction, but would also be expected to suffer from significant signal loss due to binding complex dissociation during wash steps. In embodiments, the composition comprises TEA. In embodiments, the composition comprises TEA, an ionic component, and optionally a surfactant.

Without being bound by theory, it is believed that the compositions and ECL coreactants herein (e.g., TEA) decrease the distance from the solid electrode surface where ECL is generated from an ECL label. This, in turn, increases the signal of bound label (which is held in close proximity to the electrode) relative to free label (which is distributed throughout the solution above an electrode). The increased signal from bound label can also be characterized in terms of “effective excitation length,” which is the maximum distance at which a free ECL label is able to be excited. The “effective excitation length” is impacted by: (1) the distance short-lived intermediates involved in the generation of ECL (e.g., oxidation product of the ECL coreactant) can diffuse from the electrode before they are depleted in a side reactions (a function of the lifetimes and diffusion constants for these intermediates); and (2) the rate at which free labels or unbound labeled reagents diffuse into the region close enough to the electrode to participate in a reaction with these reactive intermediates (a function of the diffusion constant for the unbound ECL labels or labeled reagents). In methods using the compositions herein, the effective excitation length is reduced by more than 2-fold, more than 3-fold, more than 4-fold, more than 5-fold, more than 9-fold, more than 7-fold, more than 8-fold, more than 9-fold, or more than 10-fold compared with a composition comprising TPA. In embodiments, the composition comprises TEA. In embodiments, the composition comprises TEA, an ionic component, and optionally a surfactant.

In embodiments, the method herein does not comprise a wash step. In embodiments where the method detects a binding complex, the method does not comprise a wash step prior to, during, or after forming a binding complex on a surface. In embodiments where the method detects an analyte of interest in a sample, the method does not comprise a wash step prior to, during, or after contacting the sample with (i) a surface comprising a binding reagent, wherein the binding reagent specifically binds to the analyte; and (ii) a detection reagent that specifically binds to the analyte. In embodiments, the method does not comprise a wash step prior to, during, or after contacting the binding complex with the composition. In embodiments, the method does not comprise a wash step prior to, during, or after a voltage to the surface to generate ECL. In embodiments, the method does not comprise a wash step prior to or during detecting the generated ECL. In embodiments, the composition comprises TEA. In embodiments, the composition comprises TEA, an ionic component, and optionally a surfactant.

In embodiments, the method herein comprises a wash step. In embodiments where the method detects a binding complex, the method comprises a wash step prior to, during, or after forming a binding complex on a surface. In embodiments where the method detects an analyte of interest in a sample, the method comprises a wash step prior to, during, or after contacting the sample with (i) a surface comprising a binding reagent, wherein the binding reagent specifically binds to the analyte; and (ii) a detection reagent that specifically binds to the analyte. In embodiments, the method comprises a wash step prior to, during, or after contacting the binding complex with the composition. In embodiments, the method comprises a wash step prior to, during, or after a voltage to the surface to generate ECL. In embodiments, the method comprises a wash step prior to or during detecting the generated ECL. In embodiments, the composition comprises TEA, BDEA, tBDEA, MDEA, DEA-PS or a combination thereof. In embodiments, the composition comprises TEA, tBDEA, MDEA, DEA-PS or a combination thereof. In embodiments, the ECL coreactant composition comprises TEA, BDEA, MDEA, DEA-PS, or a combination thereof. In embodiments, the ECL coreactant composition comprises TEA.

In embodiments, the disclosure provides a method for detecting a binding complex, comprising: (a) forming an assay mixture by combining a sample with: (i) an ECL coreactant composition or a TEA composition provided herein; and (ii) a detection mixture comprising at least two copies of a detection reagent, wherein each copy of the detection reagent comprises an ECL label; (b) contacting the assay mixture with a binding reagent immobilized on a surface, wherein the surface optionally comprises an electrode, under conditions wherein (I) a binding complex is formed on the surface, the binding complex comprising the binding reagent and a first copy of the detection reagent; and (II) a second copy of the detection reagent remains in solution; (c) applying a voltage to the surface to generate ECL; and (d) detecting the generated ECL, thereby detecting the binding complex. In embodiments, the surface comprises an electrode. In embodiments, the second copy of the detection reagent is not removed prior to any of steps (b) to (d). In embodiments, the second copy of the detection reagent is not removed prior to step (b). In embodiments, the ECL coreactant composition comprises TEA, tBDEA, MDEA, DEA-PS, or combination thereof. In embodiments, the ECL coreactant composition comprises TEA, BDEA, MDEA, DEA-PS, or a combination thereof. In embodiments, the ECL coreactant composition comprises TEA. In embodiments, the ECL coreactant composition comprises TEA, BDEA, tBDEA, MDEA, DEA-PS or a combination thereof. In embodiments, the TEA composition comprises TEA, an ionic component, and optionally a surfactant.

In embodiments, the disclosure provides a method for detecting a binding complex, comprising: (a) incubating an assay mixture comprising (i) a binding reagent immobilized on a surface, wherein the surface optionally comprises an electrode; and (ii) a detection mixture comprising at least two copies of a detection reagent, wherein each copy of the detection reagent comprises an electrochemiluminescence (ECL) label; under conditions wherein (i) a binding complex is formed on the surface, the binding complex comprising the binding reagent and a first copy of the detection reagent; and (ii) a second copy of the detection reagent remains in solution; (b) contacting the binding complex with an ECL coreactant composition or a TEA composition provided herein; (c) applying a voltage to the surface to generate ECL; and (d) detecting the generated ECL, thereby detecting the binding complex. In embodiments, the surface comprises an electrode. In embodiments, the method further comprises washing the surface prior to any of steps (b) to (d), thereby removing the second copy of the detection reagent. In embodiments, the second copy of the detection reagent is not removed prior to any of steps (b) to (d). In embodiments, the second copy of the detection reagent is not removed prior to step (b). In embodiments, the ECL coreactant composition comprises TEA, tBDEA, BDEA, MDEA, DEA-PS, or combination thereof. In embodiments, the ECL coreactant composition comprises TEA, tBDEA, MDEA, DEA-PS or a combination thereof. In embodiments, the ECL coreactant composition comprises TEA, BDEA, MDEA, DEA-PS, or a combination thereof. In composition comprises TEA, an ionic component, and optionally a surfactant.

In embodiments, the disclosure provides a method for detecting a binding complex, comprising: (a) incubating an assay mixture comprising (i) a binding reagent immobilized on a surface, wherein the surface optionally comprises an electrode; (ii) a detection mixture comprising at least two copies of a detection reagent, wherein each copy of the detection reagent comprises an electrochemiluminescence (ECL) label; and (iii) an ECL coreactant composition or a TEA composition provided herein; under conditions wherein (i) a binding complex is formed on the surface, the binding complex comprising the binding reagent and a first copy of the detection reagent; and (ii) a second copy of the detection reagent remains in solution; (b) applying a voltage to the surface to generate ECL; and (c) detecting the generated ECL, thereby detecting the binding complex. In embodiments, the surface comprises an electrode. In embodiments, the method further comprises washing the surface prior to any of steps (b) or (c), thereby removing the second copy of the detection reagent. In embodiments, the second copy of the detection reagent is not removed prior to any of steps (b) or (d). In embodiments, the second copy of the detection reagent is not removed prior to step (b). In embodiments, the ECL coreactant composition comprises TEA, tBDEA, BDEA, MDEA, DEA-PS, or combination thereof. In embodiments, the ECL coreactant composition comprises TEA, tBDEA, MDEA, DEA-PS or a combination thereof. In embodiments, the ECL coreactant composition comprises TEA, BDEA, MDEA, DEA-PS, or a combination thereof. In embodiments, the ECL coreactant composition comprises TEA. In embodiments, the TEA composition comprises TEA, an ionic component, and optionally a surfactant.

In embodiments, the binding complex further comprises an analyte, and the binding reagent and the first copy of the detection reagent each specifically binds to the analyte.

In embodiments, at least two copies of the binding reagent are immobilized on the surface, and wherein a first copy of the binding reagent forms a complex with the first copy of the detection reagent, and a second copy of the binding reagent binds to a competitor such that the second copy of the binding reagent does not form a complex with the second copy of the detection reagent. In embodiments, at least two copies of the binding reagent are immobilized on the surface, and wherein a first copy of the binding reagent forms a complex with the first copy of the detection reagent, and the second copy of the detection reagent binds to a competitor such that the second copy of the binding reagent does not form a complex with the second copy of detection reagent. Competitors and competitive assay formats are further described herein.

In embodiments, the binding reagent binds to the first copy of the detection reagent to form the binding complex.

In embodiments, the binding reagent comprises an antibody or antigen-binding fragment thereof, antigen, ligand, receptor, oligonucleotide, hapten, epitope, mimotope, or aptamer. In embodiments, the binding reagent is an antibody or a variant thereof, including an antigen/epitope-binding portion thereof, an antibody fragment or derivative, an antibody analogue, an engineered antibody, or a substance that binds to antigens in a similar manner to antibodies. In embodiments, the binding reagent comprises at least one heavy or light chain complementarity determining region (CDR) of an antibody. In embodiments, the binding reagent comprises at least two CDRs from one or more antibodies. In embodiments, the binding reagent is an antibody or antigen-binding fragment thereof. In embodiments, the binding reagent specifically binds to the analyte. As used herein, “specifically binds” means that a reagent (e.g., the binding reagent) preferentially binds to its binding partner (e.g., an epitope of the analyte) relative to a random, unrelated substance. In embodiments, the binding reagent is an antibody or antigen-binding fragment thereof, comprising a binding domain that specifically binds to an epitope of the analyte.

In embodiments, the binding reagent is immobilized to a surface. In embodiments, the binding reagent is directly immobilized to the surface. In embodiments, the binding reagent is indirectly immobilized on the surface via secondary binding partners on the binding reagent and the surface. Exemplary secondary binding partners include, but are not limited to, complementary oligonucleotides, a receptor-ligand pair, an antigen-antibody pair, a hapten-antibody pair, an epitope-antibody pair, a mimotope-antibody pair, an aptamer-target molecule pair, hybridization partners, an intercalator-target molecule pair, cross-reactive moieties (such as, e.g., thiol and maleimide or iodoacetamide; aldehyde and hydrazide; or azide and alkyne or cycloalkyne).

In embodiments, the detection reagent comprises an antibody or antigen-detection fragment thereof, antigen, ligand, receptor, oligonucleotide, hapten, epitope, mimotope, or aptamer. In embodiments, the detection reagent is an antibody or a variant thereof, including an antigen/epitope-detection portion thereof, an antibody fragment or derivative, an antibody analogue, an engineered antibody, or a substance that binds to antigens in a similar manner to antibodies. In embodiments, the detection reagent comprises at least one heavy or light chain complementarity determining region (CDR) of an antibody. In embodiments, the detection reagent comprises at least two CDRs from one or more antibodies. In embodiments, the detection reagent is an antibody or antigen-detection fragment thereof. In embodiments, the detection reagent specifically binds to the analyte. In embodiments, the detection reagent is an antibody or antigen-binding fragment thereof, comprising a binding domain that specifically binds to an epitope of the analyte. In embodiments, the detection reagent binds to a different epitope of the analyte than the binding reagent. In embodiments, both the binding reagent and the detection reagent are antibodies or antigen-binding fragments thereof.

In embodiments, the detection reagent comprises an ECL label. In embodiments, the ECL label comprises an electrochemiluminescent organometallic complex. In embodiments, the organometallic complex comprises ruthenium, osmium, iridium, rhenium, and/or a lanthanide metal. In embodiments, the organometallic complex comprises a substituted or unsubstituted bipyridine or a substituted or unsubstituted phenanthroline. In embodiments, the ECL label comprises ruthenium. In embodiments, the ECL label comprises ruthenium (II) tris-bipyridine. In embodiments, the ECL label comprises a substituted bipyridine. In embodiments, the ECL label comprises an organometallic complex comprising at least one substituted bipyridine ligand, wherein the substituted bipyridine ligand comprises at least one sulfonate group. In embodiments, the ECL label comprises an organometallic complex comprising at least two substituted bipyridine ligands, wherein each substituted bipyridine ligand comprises at least one sulfonate group. In embodiments, the substituted bipyridine ligand comprising at least one sulfonate group is a compound of Formula I. In embodiments, the ECL label comprises a compound of Formula II. Exemplary ECL labels are provided in U.S. Pat. Nos. 5,714,089; 6,136,268; 6,316,607; 6,468,741; 6,479,233; 6,808,939; and 9,499,573, each of which are herein incorporated by reference in its entirety.

In embodiments, the binding reagent and/or the detection reagent bind directly to the analyte. For example, the binding reagent and the detection reagent are each an antibody or antigen-binding fragment thereof that binds specifically to an epitope on the analyte. In embodiments, the binding reagent and/or the detection reagent indirectly bind the analyte via a secondary interaction. In embodiments, the analyte is linked to a binding partner of the binding reagent and/or the detection reagent. For example, the binding reagent and/or the detection reagent comprise streptavidin, and the analyte is linked to biotin. Further examples of binding partners that can be recognized through secondary interactions include, e.g., avidin-biotin, streptavidin-biotin, antibody-hapten, antibody-epitope tag, nucleic acid-complementary nucleic acid, aptamer-aptamer target, and receptor-ligand.

In embodiments, the surface comprises a multi-well plate. In embodiments, the surface comprises a particle. In embodiments, the surface comprises an assay cartridge. In embodiments, the surface comprises a surface of a slide, a chip, a well, an assay cell or a flow cell, a tube, a channel, a bead, or a microparticle. In embodiments, the surface comprises a particle, and the method further comprises collecting the particle on an additional surface, and applying the voltage to the particle on the additional surface. In embodiments, the particle is a bead (such as a magnetic bead), and the method further comprises collecting the bead(s) on a magnetized plate, wherein the plate comprises an electrode, and applying the voltage to the plate. In embodiments, the surface and/or additional surface comprises an electrode. In embodiments, the electrode is a carbon electrode, a platinum electrode, a gold electrode, or a silver electrode. In embodiments, the electrode is a carbon ink electrode. In embodiments, the substrate surface comprises an electrode. In embodiments, the substrate comprises an electrode layer coated thereon.

In embodiments, the method comprises measuring the amount of an analyte of interest or a binding complex in a sample. Approaches to using a measured amount of ECL signal to determine the quantity and/or concentration of an ECL label (or an analyte or binding complex) in an ECL-based binding assay are known to one of ordinary skill in the art and include, for example, using a calibration standard and/or calibration curve to establish the relationship between ECL signal and quantity and/or concentration of the ECL label and/or analyte. Calibration may be performed at different times, for example, during development of a method, during qualification of a specific lot of assay materials, and/or at the time of an assay measurement. Calibration may also be performed using calculations based on the known physical and chemical behaviors of the assay components and instrumentation.

The methods herein can be used to test a variety of samples that may contain an analyte of interest. In embodiments, the sample is a biological sample. In embodiments, the sample is derived from a cell (live or dead), immortalized cell, cell-derived product, cell fragment, cell fraction, cell lysate (fractionated or unfractionated), eukaryotic cell, prokaryotic cell, organelle, cell nucleus and fractions thereof, cell membrane, hybridoma, cell culture supernatant (e.g., supernatant from an antibody-producing organism such as a hybridoma), cytoskeleton, protein complexes, structural biological components, skeletal components such as ligaments and tendons, hair, fur, feathers, hair fractions, skin, dermis, endodermis, mammalian fluid, secretion, excretion, whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions and excretions, mucosal swabs, tissue aspirates, tissue homogenates, or an extraction, purification therefrom, or dilution thereof. In embodiments, the sample is derived from a plant, plant byproduct, soil, water source, oil, sewage, or environmental sample. In embodiments, the sample further comprises water, an organic solvent (e.g., acetonitrile, dimethyl sulfoxide, dimethyl formamide, n-methyl-pyrrolidone, alcohol, or combination thereof), EDTA, heparin, citrate, or combination thereof. Samples may be obtained from a single source described herein, or may contain a mixture from two or more sources.

Analytes that can be measured using the methods of the disclosure include, but are not limited to, whole cells, cell surface antigens, subcellular particles (e.g., organelles or membrane fragments), exosomes, extracellular vesicles, liposomes, membrane vesicles, viruses, prions, dust mites or fragments thereof, viroids, antibodies, antigens, haptens, fatty acids, nucleic acids (and synthetic analogs), proteins (and synthetic analogs), lipoproteins, polysaccharides, inhibitors, cofactors, haptens, cell receptors, receptor ligands, lipopolysaccharides, glycoproteins, peptides, polypeptides, enzymes, enzyme substrates, enzyme products, second messengers, cellular metabolites, hormones, pharmacological agents, synthetic organic molecules, organometallic molecules, tranquilizers, barbiturates, alkaloids, steroids, vitamins, amino acids, sugars, lectins, recombinant or derived proteins, biotin, avidin, streptavidin, or inorganic molecules present in the sample. Activities that may be measured include, but are not limited to, the activities of phosphorylases, phosphatases, esterases, trans-glutaminases, nucleic acid damaging activities, transferases, oxidases, reductases, dehydrogenases, glycosidases, ribosomes, protein processing enzymes (e.g., proteases, kinases, protein phosphatases, ubiquitin-protein ligases, etc.), nucleic acid processing enzymes (e.g., polymerases, nucleases, integrases, ligases, helicases, telomerases, etc.), cellular receptor activation, second messenger system activation, etc.

Whole cells may be animal, plant, or bacteria, and may be viable or dead. Examples include plant pathogens such as fungi and nematodes. The term “subcellular particles” has its plain and ordinary meaning as understood in light of the specification, and encompasses, for example, subcellular organelles, membrane particles as from disrupted cells, fragments of cell walls, ribosomes, multi-enzyme complexes, and other particles which can be derived from living organisms. Nucleic acids include, for example, chromosomal DNA, plasmid DNA, viral DNA, and recombinant DNA derived from multiple sources. Nucleic acids also include RNA, for example messenger RNA, ribosomal RNA and transfer RNA. Polypeptides include, for example, enzymes, transport proteins, receptor proteins, and structural proteins such as viral coat proteins. In embodiments, the polypeptide is an enzyme or an antibody. In embodiments, the polypeptide is a monoclonal antibody. Hormones include, for example, insulin and T4 thyroid hormone. Pharmacological agents include, for example, cardiac glycosides. It is within the scope of this disclosure to include synthetic substances which chemically resemble biological materials, such as synthetic polypeptides, synthetic nucleic acids, and synthetic membranes, vesicles and liposomes. The foregoing is not intended to be a comprehensive list of the biological substances suitable for use in this disclosure, but is meant only to illustrate the wide scope of the disclosure.

In embodiments, the method herein is a multiplexed method capable of detecting multiple binding complexes and/or analytes. In embodiments, the multiplexed method simultaneously detects multiple binding complexes and/or analytes. In embodiments, the multiplexed method comprises repeating one or more method steps to measure the multiple binding complexes and/or analytes. In embodiments, each of the method steps is performed for each binding complex and/or analyte in parallel. In embodiments where the method detects multiple binding complexes, each binding complex comprises a different binding and/or detection reagent. In embodiments where the method detects multiple analytes, each analyte binds to different binding and/or detection reagents. In embodiments, the binding of each analyte to its corresponding binding reagent is performed in parallel by contacting the surface(s) with a sample comprising multiple analytes.

In embodiments, the multiplexed method does not comprise a wash step. Multiplexed non-wash assays are particularly challenging due to the increased amount of detection reagent present in the assay mixture, and therefore increased amount of ECL label in solution, contributing to a high background ECL signal. The ECL coreactants herein had surprisingly good discrimination between bound ECL label and free ECL label in multiplexed assay formats, including multiplexed non-wash assays, providing high ECL signal and low background. In embodiments, the ECL coreactant is TEA.

In embodiments, the surface comprises a plurality of binding domains, and each binding complex is formed in a different binding domain. In embodiments, the plurality of binding domains is on a single surface. In embodiments, the surface comprises a multi-well plate, and each binding domain is in a different well. In embodiments, the surface comprises a well of a multi-well plate, and each binding domain is in a separate portion of the well. In embodiments, the plurality of binding domains is on one or more surfaces. In embodiments, the surface comprises a particle, and each binding domain is on a different particle. In embodiments, the particles are arranged in a particle array. In embodiments, the particles are coded to allow for identification of specific particles and distinguish between each binding domain

In embodiments, each binding domain comprises a targeting agent capable of binding to a targeting agent complement, and each binding reagent comprises a supplemental linking agent capable of binding to a linking agent. In embodiments, the binding reagent is immobilized in the binding domain by: (1) binding the binding reagent, via the supplemental linking agent, to a targeting reagent complement connected to the linking agent; and (2) binding the product of (1) to the binding domain comprising the targeting agent, wherein (i) each binding domain comprises a different targeting agent, and (ii) each targeting reagent complement selectively binds to one of the targeting reagents, thereby immobilizing each binding reagent to its associated binding domain.

In embodiments, an optional bridging agent, which is a binding partner of both the linking agent and the supplemental linking agent, bridges the linking agent and supplemental linking agent, such that the binding reagents, each bound to its respective targeting agent complement, are contacted with the binding domains and bind to their respective targeting agents via the bridging agent, the targeting agent complement on each of the binding reagents, and the targeting agent on each of the binding domains.

In embodiments, the targeting agent and targeting agent complement are two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the targeting agent and targeting agent complement are cross-reactive moieties, e.g., thiol and maleimide or iodoacetamide; aldehyde and hydrazide; or azide and alkyne or cycloalkyne. In embodiments, the targeting agent is biotin, and the targeting agent complement is avidin or streptavidin.

In embodiments, the linking agent and supplemental linking agent are two members of a binding partner pair selected from avidin-biotin, streptavidin-biotin, antibody-hapten, antibody-antigen, antibody-epitope tag, nucleic acid-complementary nucleic acid, aptamer-aptamer target, and receptor-ligand. In embodiments, the linking agent and supplemental linking agent are cross-reactive moieties, e.g., thiol and maleimide or iodoacetamide; aldehyde and hydrazide; or azide and alkyne or cycloalkyne. In embodiments, the linking agent is avidin or streptavidin, and the supplemental linking agent is biotin. In embodiments, the targeting agent and targeting agent complement are complementary oligonucleotides. In embodiments, the targeting agent complement is streptavidin, the targeting agent is biotin, and the linking agent and the supplemental linking agent are complementary oligonucleotides.

In embodiments comprising a bridging agent, the bridging agent is streptavidin or avidin, and the linking agents and the supplemental linking agents are each biotin.

In embodiments, the disclosure provides a method for producing a composition comprising combining: an ECL coreactant, an ionic component, and a surfactant. In embodiments, the ECL coreactant is tributylamine (TBA), (dibutyl)aminoethanol (DBAE), (diethyl)aminoethanol (DEAE), triethanolamine (TEA), butyldiethanolamine (BDEA), propyldiethanolamine (PDEA), ethyldiethanolamine (EDEA), methyldiethanolamine (MDEA), tert-butyldiethanolamine (tBDEA), dibutylamine (DBA), butylethanolamine (BEA), diethanolamine (DEA), dibutylamine propylsulfonate (DBA-PS), dibutylamine butylsulfonate (DBA-BS), butylethanolamine propylsulfonate (BEA-PS), butylethanolamine butylsulfonate (BEA-BS), diethanolamine propylsulfonate (DEA-PS), or diethanolamine butylsulfonate (DEA-BS, also known as 3-[Bis-(2-hydroxy-ethyl)-amino]-propane-1-sulfonic acid).

In embodiments, the disclosure further provides a method for producing a composition comprising combining: triethanolamine (TEA) and an ionic component. In embodiments, the disclosure further provides a method for producing a composition comprising combining: triethanolamine (TEA), an ionic component, and a surfactant, wherein the method does not comprise adding an additional pH buffering component. In embodiments, one or more of the components is provided in dry form. Ionic components and surfactants suitable for the composition are provided herein and include, e.g., NaCl, KCl, and LiCl (for the ionic component), and Poloxamer 407 (KOLLIPHOR® P-407), PEO₁₈-PPO₇₂-PEO₁₈ (PLURONIC® P-123), PEG₅-PPG₆₈-PEG₅(PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, and an alkyl ether-polyethylene glycol (e.g., PEG(18) tridecyl ether) (for the surfactant). The TEA, ionic component, and surfactant can be included at the concentrations described herein. In embodiments, the composition produced by the method comprises about 1000 mM to about 6500 mM TEA, about 700 to about 1000 mM ionic component, and about 0.5 mM to about 10 mM surfactant. In embodiments, the composition produced by the method comprises about 1200 mM to about 1600 mM TEA, about 700 to about 1000 mM ionic component, and about 1 mM to about 5 mM surfactant.

In embodiments, the disclosure further provides a method for producing a composition comprising combining: tert-butyldiethanolamine (tBDEA), methyldiethanolamine (MDEA), [Bis-(2-hydroxy-ethyl)-amino]-propane-1-sulfonic acid (DEA-PS), or a combination thereof; an ionic component; and a surfactant. In embodiments, one or more of the components is provided in dry form. Ionic components and surfactants suitable for the composition are provided herein and include, e.g., NaCl, KCl, and LiCl (for the ionic component), and Poloxamer 407 (KOLLIPHOR® P-407), PEO₁₈-PPO₇₂-PEO₁₈ (PLURONIC® P-123), PEG₅-PPG₆₈-PEG₅ (PLURONIC® L-121), PPO₂₆-PEO₅-PPO₂₆ (PLURONIC®31R1), ethylenediamine tetrakis(propoxylate-block-ethoxylate) tetrol (TETRONIC® 701), polyethylene glycol dodecyl ether (BRIJ® L4), polyethylene glycol hexadecyl ether (BRIJ® 58), polysorbate 20 (TWEEN® 20), 2,4,7,9-tetramethyl-d-decyne-4,7-diol ethoxylate, and an alkyl ether-polyethylene glycol (e.g., PEG(18) tridecyl ether) (for the surfactant). The tBDEA and/or MDEA, ionic component, and surfactant can be included at the concentrations described herein. In embodiments, the composition produced by the method comprises about 50 mM to about 250 mM tBDEA, about 50 mM to about 250 mM MDEA, and/or about 50 mM to about 250 mM DEA-PS, about 700 to about 1000 mM ionic component, and about 0.5 mM to about 10 mM surfactant.

### Methods Utilizing an ECL-Labeled Oligonucleotide Probes

In some embodiments of the methods disclosed herein, including those described above, the method is a new type of molecular assay for sensitive and specific detection based on the use of an ECL-labeled oligonucleotide probe. In embodiments, the probe comprises a quenching moiety that quenches ECL signal at least when the probe is not hybridized to a complementary oligonucleotide. In embodiments, the probe has a molecular beacon-like design. Without being bound by any theory, it is believed that ECL signal is most efficiently generated close to the surface of the electrode. In embodiments, immobilizing the ECL-labeled oligonucleotide probe on or near the electrode surface results in a higher signal compared to detection of the ECL-labeled oligonucleotide probe in solution. When the target oligonucleotide to which the ECL-labeled oligonucleotide probe hybridizes is immobilized to the surface, the ECL-labeled oligonucleotide probe will be within optimal excitation distance. In addition, when the probe comprises a quenching moiety, the method comprises a step to dequench the probes that are hybridized to a complementary oligonucleotide, while not dequenching the probes which are not hybridized to a complementary oligonucleotide, referred to as selective dequenching. Both immobilization and dequenching will result in an increase in signal. In embodiments, the selective dequenching comprises hybridizing an ECL-labeled oligonucleotide having a molecular beacon-like design to a complementary oligonucleotide, such that the quenching moiety is no longer in proximity to the ECL label. In embodiments, the selective dequenching comprises cleaving the quenching moiety only from probes which are hybridized to a complementary oligonucleotide thereby releasing the quenching moiety into solution, and leaving the ECL-labeled probe hybridized to the complementary oligonucleotide.

In embodiments, the ECL-labeled oligonucleotide probe assay can be combined with a read buffer ECL co-reactant that more selectively excites ECL label closer to the surface of the electrode to further enhance the signal from specific binding events TEA exemplifies this more selective co-reactant, but this group of co-reactants also includes MDEA, BDEA, tBDEA, DEA-PS, or combinations thereof. In embodiments, the group of co-reactants also includes MDEA, tBDEA, DEA-PS, or combinations thereof. Without being bound by any theory, it is believed that in this group of co-reactants, exemplified by TEA, the radical has a short lifetime resulting in ECL generation only very close to the electrode surface and not in the solution of the well, in contrast to more long-lived species such as TPA. By immobilizing the ECL-labeled oligonucleotide probe to the surface by hybridization to an immobilized complementary oligonucleotide (for example, as illustrated in FIGS. 14, 20 and 21), the majority of the signal will be generated from specifically bound molecular species. The small fraction of co-reactant molecules diffusing within excitation distance to activate the ECL label on the ECL-labeled oligonucleotide probes that are not hybridized to a complementary oligonucleotide will generate an ECL signal that is quenched, (e.g., because the molecular beacon-like probe is not in its open format (e.g., as illustrated in FIGS. 14 and 20) or because the quenching moiety is in proximity to the ECL label (e.g., as illustrated in FIG. 21)), and will therefore generate less ECL signal.

The low background resulting from using ECL-labeled oligonucleotide probe comprising a quenching moiety which is selectively dequenched only when hybridized to a complementary oligonucleotide enables a wash-free assay where the probe can be added to the read buffer and applied to an immobilized target sequence before the plate is read. Without being bound by any theory, it is believed that one or more of the following factors, particularly an assay having all three factors, contribute to increased assay performance and enable an assay that does not require a wash step to remove ECL-labeled oligonucleotide probe that is not hybridized to the target oligonucleotide prior to detection of ECL signal:
1 Immobilization of the ECL-labeled oligonucleotide probe near the surface of the electrode enables more efficient ECL signal generation;
2. Selective dequenching of the ECL-labeled oligonucleotide probe hybridized to an immobilized complementary oligonucleotide selectively increases the ECL signal generated for specific binding events; and/or
3. A TEA-based read buffer (or co-reactant with a similar short radical lifetime, e.g. MDEA, BDEA, tBDEA, and DEA-PS) further enhances the discrimination in signal generation between ECL-labeled oligonucleotide probe hybridized to the immobilized complementary oligonucleotide and quenched ECL-labeled oligonucleotide probe in solution.

In embodiments, methods comprise using an ECL-labeled oligonucleotide probe as described herein to detect the presence of a binding complex, a binding partner, and/or an analyte. In embodiments, the method comprises providing a substrate (for example a well, multi-well plate, slide, etc.) having a surface with a binding reagent and/or binding partner, and/or binding complex, as disclosed herein immobilized thereon. In embodiments, the substrate surface comprises an electrode. In embodiments, the substrate comprises an electrode layer coated thereon.

In embodiments, the immobilized binding reagent is exposed to a composition that contains a binding partner and/or binding complex for the binding reagent. For example, the binding partner and/or binding complex is or comprises an analyte of interest. The binding reagent is permitted to bind the binding partner and/or binding complex. Following binding of the binding partner and/or binding complex, optionally a wash can be performed to wash unbound binding partner and/or binding complex. In embodiments, the binding partner and/or binding complex comprises an oligonucleotide. The binding partner and/or binding complex can comprise the oligonucleotide prior to being exposed to the binding reagent, or the oligonucleotide can be added to the binding partner and/or binding complex after it is bound by the binding reagent. For example, the binding partner and/or binding complex could be labeled with the oligonucleotide prior to being exposed to the binding reagent, or after the binding partner and/or binding complex is bound to the binding reagent. In embodiments, the binding partner is an oligonucleotide analyte of interest, and thus the binding partner and oligonucleotide are one and the same. In embodiments, the binding partner and/or binding complex comprises an oligonucleotide analyte, but further comprises an additional oligonucleotide (e.g. a tag sequence) that is added to or hybridized with the oligonucleotide analyte.

The binding partner and/or binding complex comprising the oligonucleotide is exposed to ECL-labeled oligonucleotide probes described herein. In embodiments, ECL-labeled oligonucleotide probes comprise an oligonucleotide sequence that is complementary to the oligonucleotide sequence of the binding partner and/or binding complex oligonucleotide. In embodiments, the binding partner and/or binding complex is immobilized on a substrate before exposure to the ECL-labeled oligonucleotide probes, and in other embodiments, the binding partner and/or binding complex is immobilized on a substrate after exposure to the ECL-labeled oligonucleotide probes. In embodiments, the ECL-labeled probes comprise a quenching moiety that quenched ECL signal at least while the probe is not hybridized to a complementary oligonucleotide. In embodiments, the ECL-labeled oligonucleotide probes include a stem-loop or hairpin structure, an ECL label, and a quenching moiety, wherein the quenching moiety is in proximity to the ECL label and quenches the ECL label when the oligonucleotide probe is in a stem-loop or hairpin configuration but does not quench the ECL label when the stem-loop or hairpin structure is in an open configuration. In embodiments, the ECL-labeled an ECL label and a quencher that are sufficiently close to each other on the probe when the probe is in a linear configuration (e.g., not in a stem-loop or hairpin configuration) that the quenching moiety quenches the ECL signal whether the probe is hybridized to a complementary oligonucleotide or not. In embodiments, the ECL-labeled oligonucleotide probes are present in the composition that contains a binding partner and/or binding complex for the binding reagent such that the binding of the binding partner and/or binding complex to the binding reagent immobilized on the substrate and exposure of the binding partner to the ECL-labeled oligonucleotide probes happen simultaneously. In embodiments, the ECL-labeled oligonucleotide probe and the binding partner and/or binding complex comprising the oligonucleotide are mixed together first, and then the composition comprising both is exposed to the substrate comprising the immobilized binding reagent. In embodiments, the ECL-labeled oligonucleotide probes are added after the binding partner and/or binding complex is bound to the substrate by the binding reagent, after the optional wash if the wash is performed.

The ECL-labeled oligonucleotide probes are permitted to hybridize to the complementary oligonucleotide of the bound binding partner or binding complex. In embodiments where the ECL-labeled oligonucleotide probes have a stem-loop or hairpin structure with an ECL label and a quenching moiety, hybridization opens the stem-loop or hairpin structure, separating the ECL label from the quenching moiety such that the quenching moiety will no longer quench an ECL signal emitted by the ECL label. Conditions for hybridization of the ECL-labeled oligonucleotide probes to the complementary oligonucleotides on the binding partner can be conditions known in the art for conventional oligonucleotide probes (e.g., molecular beacon probes or hydrolysis probes (also referred to as TaqMan® probes)).

In embodiments, the hybridized ECL-labeled oligonucleotide probes are selectively dequenched, leaving unhybridized probes quenched. In embodiments, the selective dequenching comprises hybridizing an ECL-labeled oligonucleotide having a molecular beacon-like design to a complementary oligonucleotide, such that the quenching moiety is no longer in proximity to the ECL label. In embodiments, the selective dequenching comprises cleaving the quenching moiety only from probes which are hybridized to a complementary oligonucleotide thereby releasing the quenching moiety into solution, and leaving the ECL-labeled probe hybridized to the complementary oligonucleotide. In embodiments, a portion of the ECL-labeled oligonucleotide probes do not hybridize to a complementary oligonucleotide and remain in the closed stem-loop or hairpin structure, or are otherwise configured such that the quenching moiety is in proximity to the ECL label and quenches ECL signal emitted by the label at least when th probe is not hybridized to a complementary oligonucleotide.

In embodiments, the selective dequenching comprises selectively cleaving the quenching moiety from only the portion of ECL-labeled probes hybridized to the oligonucleotide of the binding partner and/or binding complex such that the quenching moiety is released into solution and is no longer in proximity to the ECL label of the hybridized ECL-labeled probe which remains hybridized to the oligonucleotide of the binding partner and/or binding complex after cleavage of the quenching moiety. In embodiments, the cleaving is performed by an enzyme, for example, a restriction endonuclease, e.g. a nicking endonuclease, an RNaseH2, or a polymerase having 5′ exonuclease activity. In embodiments, the enzyme cleaves only the ECL-labeled oligonucleotide probe leaving the oligonucleotide of the binding partner and/or binding complex intact. In embodiments, the enzyme is a nicking restriction endonuclease that recognizes a sequence in the hybridized ECL-labeled probe, or an RNaseH2 which recognizes an RNA base in the hybridized ECL-labeled probe. In embodiments, the enzyme is a polymerase having 5′ exonuclease activity, and the method comprises hybridizing a primer to a complementary sequence on a portion of the oligonucleotide of the binding partner and/or binding complex at a position 5′ of the hybridized ECL-labeled probe, performing an extension reaction such that the polymerase having 5′ exonuclease activity (e.g. a TaqMan polymerase) extends the primer until the polymerase encounters the hybridized ECL-labeled probe, at which point the 5′ exonuclease activity of the polymerase cleaves the quenching moiety of the hybridized ECL-labeled probe. To prevent the digestion of the entire ECL-labeled probe, the ECL-labeled probe comprises a portion that is resistant to the 5′ exonuclease activity, such that a sufficient portion of the ECL-labeled probe comprising the ECL label remains intact and hybridized to the oligonucleotide of the binding partner and/or binding complex.

The substrated comprising selectively dequenched hybridized ECL-labeled oligonucleotide probes (as well as quenched unhybridized probes in solution) are exposed to an ECL co-reactant as described herein. The ECL co-reactant can be added to the composition prior to hybridization or after. In embodiments, the ECL co-reactant is in a composition (e.g., read buffer) as described herein. The ECL co-reactant, the ECL-labeled oligonucleotide probes, and the binding partner can all be in separate compositions (e.g., solutions), such that they can be contacted with the substrate sequentially in any order (e.g. first binding partner, then ECL-labeled oligonucleotide, then ECL co-reactant last; or first ECL-labeled oligonucleotide, then ECL-labeled oligonucleotide, then binding partner last, etc.), or they can be combined in one or more compositions (e.g. binding partner in a first composition, and the ECL-labeled oligonucleotide probes and ECL co-reactant in a second solution), such that they can be contacted with the substrate all at once, or sequentially.

The substrate and composition comprising the binding reagent, the binding partner, the hybridized and un-hybridized portions of the ECL-labeled oligonucleotide probes, and ECL co-reactant, are subjected to an ECL reaction by application of a voltage to the electrode, and any generated ECL signal is measured.

In embodiments, no wash of the substrate is conducted after the ECL-labeled oligonucleotide probes are added to the composition contacting the substrate prior to detection of the signal. This method is advantageous as it simplifies the method by eliminating at least one wash prior to detection of the ECL signal. In embodiments, no wash of the substrate is conducted after the substrate is contacted with the binding partner and prior to detection of the ECL signal. In embodiments involving no wash, either after the the ECL-labeled oligonucleotide probes are added, or after the substrate is contacted with the binding partner and/or binding complex, the ECL co-reactant is selected from the group consisting of TEA, BDEA, tBDEA, MDEA, and DEA-PS, or combinations thereof. In embodiments, the co-reactant is TEA. In embodiments involving no wash, either after the ECL-labeled oligonucleotide probes are added, or after the substrate is contacted with the binding partner and/or binding complex, the ECL co-reactant is not TPA. The combination of the use of ECL-labeled oligonucleotide probes having a quenching moiety (e.g., having stem-loop or hairpin structures, (e.g., ECL-labeled molecular beacon probes), or other structures where the quenching moiety is in proximity to the ECL label), binding partners and/or binding complex immobilized on the substrate, and ECL co-reactant compositions comprising TEA, BDEA, tBDEA, MDEA, and DEA-PS, or combinations thereof, and TEA in particular, yield a superior ECL signal even when a wash to remove unhybridized ECL-labeled oligonucleotide probes is not performed.

In embodiments, the binding partner comprises an analyte and/or binding complex. In embodiments, the analyte comprises a peptide, and/or analyte comprises an oligonucleotide. In embodiments, analyte is the oligonucleotide of the binding partner. In embodiments, the analyte is labeled with the oligonucleotide. In embodiments, the analyte is labeled with the oligonucleotide by binding the analyte with a detection reagent comprising the oligonucleotide, optionally to form a binding complex. In embodiments, the oligonucleotide of the binding partner and/or binding complex comprises multiple copies of the sequence complementary to the oligonucleotide sequence of the ECL-labeled oligonucleotide probes. In embodiments, prior to contacting the substrate with the plurality of the ECL-labeled oligonucleotide probes, an amplification reaction is performed to generate the multiple copies of the sequence complementary to the oligonucleotide sequence of the ECL-labeled oligonucleotide probes. In embodiments, the amplification reaction is a rolling circle amplification reaction. In embodiments, the binding partner and/or binding complex comprises an oligonucleotide primer for the rolling circle amplification.

In embodiments, the binding partner and/or binding complex comprises an analyte. In embodiments, the analyte comprises a peptide, and/or analyte comprises an oligonucleotide. In embodiments, analyte is the oligonucleotide of the binding partner. In embodiments, the analyte is labeled indirectly with an oligonucleotide primer or an extended oligonucleotide primer. In embodiments, the analyte is a peptide and is labeled with the oligonucleotide primer by binding the analyte with a detection reagent (such as an antibody) comprising the oligonucleotide primer, optionally to form a binding complex. In embodiments, the oligonucleotide primer that labels the peptide analyte is extended after labeling the analyte. The extended oligonucleotide primer comprises multiple copies of the sequence complementary to the oligonucleotide sequence of the ECL-labeled probes. In embodiments, prior to contacting the substrate with the plurality of the ECL-labeled probes, an amplification reaction is performed extend the oligonucleotide primer and generate the multiple copies of the sequence complementary to at least a portion of the oligonucleotide sequence of the ECL-labeled probes. In embodiments, the amplification reaction is a rolling circle amplification reaction. In embodiments, the binding complex comprises an analyte labeled with an extended oligonucleotide primer.

In embodiments, the disclosure provides a method for detecting an analyte of interest in a sample, comprising: (a) contacting the sample with: (i) a surface comprising a binding reagent, wherein the binding reagent specifically binds to the analyte; and (ii) a detection reagent that specifically binds to the analyte, wherein the detection reagent comprises an electrochemiluminescence (ECL) label, thereby forming a binding complex on the surface comprising the binding reagent, the analyte, and the detection reagent; (b) contacting the binding complex on the surface with an ECL coreactant composition or a TEA composition provided herein; (c) applying a voltage to the surface to generate ECL; and (d) detecting the generated ECL, thereby detecting the analyte. In embodiments, the ECL coreactant composition comprises TEA, BDEA, tBDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the ECL coreactant composition comprises TEA. In embodiments, the TEA composition comprises TEA, an ionic component, and optionally a surfactant. In embodiments, the surface comprises an electrode. In embodiments, the analyte of interest is an oligonucleotide, and the detection reagent that specifically binds to the analyte is an oligonucleotide probe that comprises an ECL label and a quenching moiety which quenches the ECL signal at least when the ECL-labeled oligonucleotide probe is not hybridized to a complementary oligonucleotide. In embodiments, the ECL-labeled oligonucleotide probe comprises a stem-loop or hairpin structure, an ECL label, and a quenching moiety, wherein said quenching moiety is in proximity to the ECL label and quenches the ECL label when the ECL-labeled oligonucleotide probe is in a stem-loop or hairpin configuration, but does not quench the ECL label when the stem-loop or hairpin structure is in an open configuration. In embodiments, the ECL-labeled oligonucleotide probe with the stem-loop or hairpin structure is an ECL-labeled molecular beacon probe. In embodiments, the ECL-labeled oligonucleotide probes comprise an ECL label and a quencher that are sufficiently close to each other on the probe when the probe is in a linear configuration (e.g., not in a stem-loop or hairpin configuration) that the quenching moiety quenches the ECL signal whether the probe is hybridized to a complementary oligonucleotide or not.

In embodiments, the disclosure provides a method for detecting an analyte of interest in a sample, comprising contacting the sample with: (i) a surface comprising a binding reagent, wherein the binding reagent specifically binds to a binding complex comprising the analyte (e.g., a peptide); (ii) a capture reagent (e.g., an antibody) that specifically binds to the analyte, wherein the capture reagent additionally comprises a binding partner to the binding reagent (e.g., biotin/streptavidin); (iii) a detection reagent that specifically binds to the analyte, wherein the detection reagent comprises an oligonucleotide primer (e.g., oligonucleotide lableled antibody); (iv) a template oligonucleotide; and (v) an ECL-labeled oligonucleotide probe. In embodiments, the capture reagent and the detection reagent bind to the analyte, the primer binds to the template oligonucleotide, and the primer is extended via amplification of the template oligonucleotide to form a binding complex comprising the capture reagent, analyte, detection reagent, and extended oligonucleotide primer (e.g., as illustrated in FIG. 20). In embodiments, the ECL-labeled oligonucleotide probe binds to the extended primer oligonucleotide of the binding complex. The surface is contacted with an ECL coreactant composition or a TEA composition provided herein, a voltage is applied to the surface to generate ECL, and the generated ECL is detected, thereby detecting the analyte. In embodiments, the ECL coreactant composition comprises TEA, BDEA, tBDEA, MDEA, DEA-PS, or combination thereof. In embodiments, the ECL coreactant composition comprises TEA, tBDEA, MDEA, DEA-PS, or a combination thereof. In embodiments, the ECL coreactant composition comprises TEA, BDEA, MDEA, DEA-PS, or a combination thereof. In embodiments, the ECL coreactant composition comprises TEA. In embodiments, the TEA composition comprises TEA, an ionic component, and optionally a surfactant. In embodiments, the surface comprises an electrode. In embodiments, the ECL-labeled oligonucleotide probe is a molecular beacon probe.

In embodiments, the binding reagent is, for example, a capture oligonucleotide, and the binding partner is an oligonucleotide of interest (analyte) that comprises a nucleic acid sequence that is complementary to the sequence of the capture oligonucleotide, as well as a sequence that is complementary to the ECL-labeled oligonucleotide probe. FIG. 14 illustrates one example of such an embodiment, wherein the ECL-labeled oligonucleotide probe comprises a stem-loop structure and a quenching moiety as described herein. As shown on the left side of FIG. 14, when the ECL-labeled oligonucleotide probe is not hybridized to the target, the ECL label (★) (e.g., Ru(bpy)₃-based S-TAG) is near the quenching moiety (●) (e.g., BHQ2 or Iowa Black), such that ECL signal is quenched. As illustrated on the right side of FIG. 14, the binding partner is a target oligonucleotide (Target) which is hybridized to a capture oligonucleotide (binding reagent) having a complementary sequence. The capture oligonucleotide (binding reagent) is immobilized on the surface of the electrode substrate (grey oval). When the ECL-labeled oligonucleotide probe hybridizes to the complementary oligonucleotide sequence of the target oligonucleotide, the stem-loop structure opens, separating the ECL label (★) from the quenching moiety (●) such that an ECL signal can be generated when a voltage is applied in the presence of an ECL co-reactant (TEA; not shown).

In embodiments, the principle illustrated in FIG. 14 is applied to other binding partners or to a binding complex. For example, the capture oligonucleotide may, instead, be an antibody or other reagent that binds an analyte such as a peptide, immobilizing the analyte on the surface of the substrate. In embodiments, the capture oligonucleotide may, instead, be streptavidin, that binds to a binding complex comprising the analyte and a capture reagent, thereby immobilizing the analyte on the surface of the substrate. The binding complex may comprise a biotinylated capture reagent, the analyte, and a detection reagent comprising an oligonucleotide. The oligonucleotide of the binding partner and/or binding complex may be an extended oligonucleotide that originates from an oligonucleotide primer that labels (either directly or indirectly) the analyte. That extended primer may comprise one or more copies of a sequence complementary to the ECL-labeled oligonucleotide probe. See, for example, FIG. 20. In FIG. 20, a peptide analyte is immobilized on the substrate surface via an immobilized streptavidin bound to biotinylated capture antibody. The peptide analyte is indirectly labeled (via binding to a second antibody, also referred to as a detection antibody) with an oligonucleotide primer. The oligonucleotide primer binds to a template oligonucleotide, after which the template oligonucleotide ends are ligated, the primer is extended (for example, with a polymerase enzyme), and the ECL-labeled oligonucleotide probe binds to the extended primer oligonucleotide. A voltage is applied in the presence of a coreactant and the ECL signal is read. In a similar embodiment, the template oligonucleotide is provided in circular form so that ligation is not necessary. In embodiments, a wash step is not performed after the ECL-labeled oligonucleotide is added to the analyte mixture and before the ECL signal is read.

In embodiments, as described herein, an ECL-labeled oligonucleotide probe having an ECL label and a quencher that are sufficiently close to each other on the probe when the probe is in a linear configuration (e.g., not in a stem-loop or hairpin configuration) that the quenching moiety quenches the ECL signal whether the probe is hybridized to a complementary oligonucleotide or not can be used. Selective dequenching comprises cleaving the quenching moiety only from probes which are hybridized to a complementary oligonucleotide thereby releasing the quenching moiety into solution, and leaving the ECL-labeled probe hybridized to the complementary oligonucleotide. FIG. 21 illustrates an embodiment utilizing such a an ECL-labeled oligonucleotide probe and method of selective dequenching. As shown on the left side of FIG. 21, the ECL label (★) (e.g., Ru(bpy)₃-based S-TAG) is near the quenching moiety (●) (e.g., BHQ2 or Iowa Black) such that ECL signal is quenched. As illustrated in the center portion of FIG. 21, the binding partner is a target oligonucleotide (Target) which is hybridized to a capture oligonucleotide (binding reagent) having a complementary sequence. The capture oligonucleotide (binding reagent) is immobilized on the surface of the electrode substrate (grey oval). When the ECL-labeled oligonucleotide probe hybridizes to the complementary oligonucleotide sequence of the target oligonucleotide, the hybridized probe and complementary oligonucleotide form a double-stranded oligonucleotide that comprises a recognition site for a cleaving enzymethe, e.g. a sequence recognized by a nicking restriction endonuclease. As illustrated on the left side of FIG. 21, the enzyme (e.g. the nicking restriction endonuclease) cleaves the quenching moiety from the ECL-labeled probe, releasing it into solution, and leaving the remaining portion of the ECL-labeled oligonucleotide probe intact and hybridized to the target. The cleaving of the quenching moiety separates the ECL label (★) from the quenching moiety (●) such that an ECL signal can be generated when a voltage is applied in the presence of an ECL co-reactant (TEA; not shown). Other methods of selectively cleaving the quenching moiety from hybridized ECL-labeled as illustrated in FIG. 21 are contemplated and described herein, including using an RNAseH2 enzyme and an RNA portion of the of the ECL-labeled oligonucleotide probe, and using a primer and polymerase having 5′ exonuclease activity with an ECL-labeled oligonucleotide probe comprising an exonuclease resistant portion.

In embodiments, the ECL-labeled oligonucleotide probes and selective cleaving of the quenching moiety described herein, for example as illustrated in FIG. 21, can be used in place of the molecular beacon probes illustrated in FIG. 20.

In embodiments, the ECL-labeled oligonucleotide probe as described herein is utilized and the non-hybridized ECL-labeled oligonucleotide probe is not removed (no washing) prior to the ECL reaction, the ECLco-reactant is selected from the group consisting of 3-(di-n-propylamino)-propanesulfonic acid; 4-(di-n-propylamino)-butanesulfonic acid; 4-[bis-(2-hydroxyethane)-amino]-butanesulfonic acid; piperidine-N-(3-propanesulfonic acid); azepane-N-(3-propanesulfonic acid); piperidine-N-(3-propionic acid) (PPA); 3-morpholino-2-hydroxypropanesulfonic acid (MOPSO); 3-morpholinepropanesulfonic acid (MOPS); N-(2-hydroxyethyl)piperazine-N-3-propanesulfonic acid (EPPS); N-(2-hydroxyethyl)piperazine-N′-3-ethanesulfonic acid (BES); piperazine-N,N′-bis(2-ethanesulfonic acid) (PIPES); triethanolamine (TEA); N-2-hydroxypiperazine-N-2-ethanesulfonic acid (HEPES); piperazine-N,N′-bis 4-butanesulfonic acid; homopiperidine-N-3-propanesulfonic acid; piperazine-N,N′bis-3-propanesulfonic acid; piperidine-N-3-propanesulfonic acid; piperazine-N-2-hydroxyethane-N-3-methylpropanoate; piperazine-N, N′-bis-3-methylpropanoate 1,6-diaminohexane-N,N,N′,N′-tetraacetic acid; N,N-bis propyl-N-4-aminobutanesulfonic acid; N-tris(hydroxymethyl)methyl-2-aminoethane sulfonic acid (TES); 1,3-bis[tris(hydroxymethyl)methylamino]propane (bis-Tris propane); 3-dimethylamino-1-propanol 3-dimethylamino-2-propanol; N,N,N′,N′-tetrapropylpropane-1,3-diamine (TPA dimer); piperazine-N,N′-bis(2-hydroxypropanesulfonic acid (POPSO) and 2-hydroxy-3-[4-(2-hydroxyethyl)piperazin-1-yl]propane-1-sulfonic acid (HEPPSO), N-butyldiethanolamine (BDEA) 2-dibutylaminoethanol (DBAE), tert-butyldiethanolamine (tBDEA), methyldiethanolamine (MDEA), 3-[Bis-(2-hydroxy-ethyl)-amino]-propane-1-sulfonic acid (DEA-PS), and combinations thereof. In embodiments, the ECL co-reactant is selected from the group consisting of TEA, BDEA, tBDEA, MDEA, DEA-PS, and combinations thereof. In embodiments, the ECL co-reactant is selected from the group consisting of TEA, tBDEA, MDEA, DEA-PS, and combinations thereof. In embodiments, the ECL co-reactant is selected from the group consisting of TEA, BDEA, MDEA, DEA-PS, and combinations thereof. In embodiments, the ECL co-reactant is TEA.

Other embodiments will be apparent to one of skill in the art in view of the present disclosure, and the knowledge of one of skill in the art.

### Assay Module

In embodiments, the disclosure provides an assay module comprising a TEA composition in dry form, wherein the TEA composition comprises TEA, an ionic component, and optionally a surfactant. In embodiments, the disclosure provides an assay module comprising an ECL coreactant composition provided herein in dry form. In embodiments, the ECL coreactant composition comprises TEA, BDEA, tBDEA, MDEA, DEA-PS, or combination thereof. In embodiments, the ECL coreactant composition comprises TEA, tBDEA, MDEA, DEA-PS or a combination thereof.

In embodiments, the assay module comprises a multi-well plate. In embodiments, the assay module comprises an assay cartridge. In embodiments, the assay module comprises a slide, a chip, a well, an assay cell or a flow cell, a tube, a channel, a bead, or a microparticle. In embodiments, the assay module comprises an electrode. In embodiments, the electrode is a carbon electrode, a platinum electrode, a gold electrode, or a silver electrode. In embodiments, the electrode is a carbon ink electrode.

In embodiments, the assay module further comprises a binding reagent in dry form. In embodiments, the assay module further comprises a detection reagent in dry form. In embodiments, the assay module further comprises a binding reagent and a detection reagent in dry form. Binding reagents and detection reagents are further described herein. In embodiments, the detection reagent comprises an ECL label.

In embodiments, the ECL label comprises an electrochemiluminescent organometallic complex. In embodiments, the organometallic complex comprises ruthenium, osmium, iridium, rhenium, and/or a lanthanide metal. In embodiments, the organometallic complex comprises a substituted or unsubstituted bipyridine or a substituted or unsubstituted phenanthroline. In embodiments, the ECL label comprises ruthenium. In embodiments, the ECL label comprises ruthenium (II) tris-bipyridine. In embodiments, the ECL label comprises a substituted bipyridine. In embodiments, the ECL label comprises an organometallic complex comprising at least one substituted bipyridine ligand, wherein the substituted bipyridine ligand comprises at least one sulfonate group. In embodiments, the ECL label comprises an organometallic complex comprising at least two substituted bipyridine ligands, wherein each substituted bipyridine ligand comprises at least one sulfonate group. In embodiments, the substituted bipyridine ligand comprising at least one sulfonate group is a compound of Formula I. In embodiments, the ECL label comprises a compound of Formula II.

### Kits

In embodiments, the disclosure comprises a kit comprising an ECL coreactant composition or a TEA composition described herein. In embodiments, the ECL coreactant composition comprises an ECL coreactant selected from tributylamine (TBA), (dibutyl)aminoethanol (DBAE), (diethyl)aminoethanol (DEAE), triethanolamine (TEA), butyldiethanolamine (BDEA), propyldiethanolamine (PDEA), ethyldiethanolamine (EDEA), methyldiethanolamine (MDEA), tert-butyldiethanolamine (tBDEA), dibutylamine (DBA), butylethanolamine (BEA), diethanolamine (DEA), dibutylamine propylsulfonate (DBA-PS), dibutylamine butylsulfonate (DBA-BS), butylethanolamine propylsulfonate (BEA-PS), butylethanolamine butylsulfonate (BEA-BS), diethanolamine propylsulfonate (DEA-PS, also known as 3-[Bis-(2-hydroxy-ethyl)-amino]-propane-1-sulfonic acid), diethanolamine butylsulfonate (DEA-BS), and a combination thereof. In embodiments, the composition comprises TEA. In embodiments, the composition comprises tBDEA. In embodiments, the composition comprises MDEA. In embodiments, the composition comprises MDEA. In embodiments, the composition comprises DEA-PS. In embodiment, the TEA composition comprises TEA, an ionic component, and optionally a surfactant.

In embodiments, the disclosure provides a kit comprising two or more components that, when mixed, form a composition as described herein. In embodiments, the disclosure provides a kit comprising, in one or more containers, vials, or compartments: (a) triethanolamine (TEA) and (b) an ionic component, wherein the kit does not comprise an additional pH buffering component. In embodiments, the disclosure provides a kit comprising, in one or more containers, vials, or compartments: (a) triethanolamine (TEA); (b) an ionic component; and (c) a surfactant, wherein the kit does not comprise an additional pH buffering component. Ionic components (e.g., NaCl, KCl, and/or LiCl), surfactants (e.g., TRITON X-100, KOLLIPHOR® P-407, PLURONIC® P-123, PLURONIC® L-121, PLURONIC®31R), TETRONIC® 701, BRIJ® L4, BRIJ® 58, TWEEN® 20, 2,4,7,9-tetramethyl-d-decyne-4,7-diol ethoxylate, and/or alkyl ether-polyethylene glycol (e.g., PEG(18) tridecyl ether)), and their concentrations are described herein.

In embodiments, the kit further comprises an assay reagent, a calibration reagent, a surface, an ECL label, or combination thereof. In embodiments, the kit comprises an assay reagent. In embodiments, the assay reagent comprises a binding reagent, a detection reagent, or both. Binding reagents and detection reagents are further described herein and include, e.g., antibody or antigen-binding fragment thereof, antigen, ligand, receptor, oligonucleotide, hapten, epitope, mimotope, or aptamer. In embodiments, the binding reagent is an antibody or antigen-binding fragment thereof. In embodiments, the detection reagent is an antibody or antigen-binding fragment thereof. In embodiments, both the binding reagent and the detection reagent are antibodies or antigen-binding fragments thereof.

In embodiments, the kit comprises an assay module described herein. In embodiments, the assay module comprises the ECL coreactant composition or TEA composition described herein in dry form. In embodiments, the kit comprises a surface. Surfaces suitable for performing the ECL-based binding assays are described herein. In embodiments, the surface comprises a multi-well plate. In embodiments, the surface comprises an assay cartridge. In embodiments, the surface comprises a particle. In embodiments, the surface comprises a surface of a slide, a chip, a well, an assay cell or a flow cell, a tube, a bead, or a microparticle. In embodiments where the surface comprises a particle, bead, or microparticle, the kit further comprises an additional surface, e.g., a plate, for collecting the particle, bead, or microparticle. In embodiments, the additional surface comprises a magnetically collectable particle, bead, or microparticle. In embodiments, the additional surface further comprises a magnetic plate. In embodiments, the surface and/or additional surface comprises an electrode. In embodiments, the electrode is a carbon electrode, a platinum electrode, a gold electrode, or a silver electrode. In embodiments, the electrode is a carbon ink electrode.

In embodiments, the binding reagent is immobilized on the surface. In embodiments, the binding reagent and the surface are provided separately in the kit, and the kit further comprises a reagent for immobilizing the binding reagent to the surface. Methods of immobilizing binding reagents to surfaces are provided herein and include, e.g., direct immobilization or indirect immobilization via secondary binding partners on the binding reagent and the surface.

In embodiments, the kit comprises an ECL label. ECL labels are further described herein and include, e.g., ruthenium-containing compounds. In embodiments, the ECL label comprises an electrochemiluminescent organometallic complex. In embodiments, the organometallic complex comprises ruthenium, osmium, iridium, rhenium, and/or a lanthanide metal. In embodiments, the organometallic complex comprises a substituted or unsubstituted bipyridine or a substituted or unsubstituted phenanthroline. In embodiments, the ECL label comprises ruthenium. In embodiments, the ECL label comprises ruthenium (II) tris-bipyridine. In embodiments, the ECL label comprises a substituted bipyridine. In embodiments, the ECL label comprises an organometallic complex comprising at least one substituted bipyridine ligand, wherein the substituted bipyridine ligand comprises at least one sulfonate group. In embodiments, the ECL label comprises an organometallic complex comprising at least two substituted bipyridine ligands, wherein each substituted bipyridine ligand comprises at least one sulfonate group. In embodiments, the substituted bipyridine ligand comprising at least one sulfonate group is a compound of Formula I. In embodiments, the ECL label comprises a compound of Formula II. In embodiments, the detection reagent comprises an ECL label. In embodiments, the detection reagent and the ECL label are provided separately in the kit, and the kit further comprises a reagent for conjugating the detection reagent to the ECL label. Methods of conjugation are known to one of skill in the art.

In embodiments, the kit comprises a calibration reagent. In embodiments, the calibration reagent comprises a known quantity of an analyte of interest. In embodiments, the calibration reagent comprises a known quantity of an ECL label. In embodiments, the kit comprises multiple calibration reagents comprising a range of concentrations of the analyte or the ECL label. In embodiments, the multiple calibration reagents comprise concentrations of the analyte or the ECL label near the upper and lower limits of quantitation for an ECL-based binding assay described herein. In embodiments, the multiple calibration reagents span the entire dynamic range of the binding assay. In embodiments, the calibration reagent is a positive control reagent. In embodiments, the calibration reagent is a negative control reagent. In embodiments, the positive or negative control reagent is used to provide a basis of comparison for the sample to be tested with the methods of the present disclosure.

In embodiments, one or more components of the kit is provided in dry form, e.g., as a lyophilized reagent. In embodiments, one or more components of the kit is provided in solution. In embodiments, the binding reagent is lyophilized. In embodiments, the binding reagent is provided in solution. In embodiments, the detection reagent is lyophilized. In embodiments, the detection reagent is provided in solution. In embodiments, the calibration reagent is lyophilized. In embodiments, the calibration reagent is provided in solution. In embodiments, the kit further comprises a liquid diluent. In embodiments, the liquid diluent reconstitutes a dry reagent. In embodiments, the liquid diluent is water. In embodiments, one or more components of the kits is provided as a concentrated stock solution, e.g., at 2×, 4×, 5×, 10×, or 20× the working concentration of the reagent.

In embodiments, the kit comprises an assay instrument, e.g., to detect ECL generated from the compositions and methods described herein. In embodiments, the kit further comprises an assay consumable, e.g., an assay module configured to contain samples and/or reagents during one or more steps of the method described herein, pipette tips and other consumables for transferring liquid samples and reagents, covers and seals for assay modules and other consumables used in an assay (e.g., tubes, cuvettes, wells, multi-well plates, cartridges, lateral flow devices, flow cells), racks for holding other assay consumables, labels (including human readable or machine readable formats such as barcodes, RFIDs, etc.) for identifying samples, or other assay consumables and media (including paper and electronic media) for providing information about the method and/or instructions for performing the method.

All references cited herein, including patents, patent applications, papers, textbooks and the like, and the references cited therein, to the extent that they are not already, are hereby incorporated herein by reference in their entirety.

### Numbered Items

Embodiments of the present disclosure include the following numbered items:
1. A composition comprising:
   (a) triethanolamine (TEA);
   (b) an ionic component; and
   (c) an electrochemiluminescence (ECL)-labeled component,
   wherein the composition has a pH of about 7.0 to about 8.0, and wherein the composition is substantially free of an additional pH buffering component.
2. A composition consisting essentially of:
   (a) triethanolamine (TEA); (b) an ionic component; and (c) a surfactant, or
   (a) triethanolamine (TEA); (b) an ionic component; (c) a surfactant; and (d) an ECL-labeled component,
   wherein the composition has a pH of about 7.0 to about 8.0, and wherein the composition is substantially free of an additional pH buffering component.
3. A composition consisting of:
   (a) triethanolamine (TEA); (b) an ionic component; and (c) a surfactant, or
   (a) triethanolamine (TEA); (b) an ionic component; (c) a surfactant; and (d) an ECL-labeled component,
   wherein the composition has a pH of about 7.0 to about 8.0.
4. A composition comprising:
   (a) about 1000 mM to about 6500 mM of triethanolamine (TEA); and
   (b) about 500 mM to about 2000 mM of an ionic component;
   wherein the composition has a pH of about 7.0 to about 8.0.
5. A composition consisting essentially of:
   (a) about 1000 mM to about 6500 mM of triethanolamine (TEA); (b) about 500 mM to about 2000 mM of an ionic component; and (c) a surfactant, or
   (a) about 1000 mM to about 6500 mM of triethanolamine (TEA); (b) about 500 mM to about 2000 mM of an ionic component; (c) a surfactant; and (d) an ECL-labeled component,
   wherein the composition has a pH of about 7.0 to about 8.0.
6. A composition consisting of:
   (a) about 1000 mM to about 6500 mM of triethanolamine (TEA); (b) about 500 mM to about 2000 mM of an ionic component; and (c) a surfactant, or
   (a) about 1000 mM to about 6500 mM of triethanolamine (TEA); (b) about 500 mM to about 2000 mM of an ionic component; (c) a surfactant; and (d) an ECL-labeled component,
   wherein the composition has a pH of about 7.0 to about 8.0.
7. A composition comprising:
   (a) triethanolamine (TEA);
   (b) an ionic component; and
   (c) an alkyl ether-polyethylene glycol (PEG);
   wherein the composition has a pH of about 7.0 to about 8.0.
8. A composition consisting essentially of:
   (a) triethanolamine (TEA); (b) an ionic component; and (c) an alkyl ether-polyethylene glycol (PEG), or
   (a) triethanolamine (TEA); (b) an ionic component; (c) an alkyl ether-polyethylene glycol (PEG); and (d) an ECL-labeled component,
   wherein the composition has a pH of about 7.0 to about 8.0.
9. A composition consisting of:
   (a) triethanolamine (TEA); (b) an ionic component; and (c) an alkyl ether-polyethylene glycol (PEG), or
   (a) triethanolamine (TEA); (b) an ionic component; (c) an alkyl ether-polyethylene glycol (PEG); and (d) an ECL-labeled component,
   wherein the composition has a pH of about 7.0 to about 8.0.
10. A composition comprising:
   (a) triethanolamine (TEA);
   (b) an ionic component; and
   (c) optionally, one or both of an ECL-labeled component and a surfactant;
   wherein the composition has a pH of about 7.0 to about 8.0; and optionally, wherein the composition is substantially free of an additional pH buffering component.
11. The composition of item 10, wherein the composition comprises the ECL-labeled component, the surfactant, or both.
12. The composition of item 10, wherein the composition is substantially free of an additional pH buffering component.
13. The composition of any of items 10-12, wherein the composition comprises:
   (a) about 1000 mM to about 6500 mM of the TEA; and
   (b) about 500 mM to about 2000 mM of the ionic component.
14. The composition of any of items 10-13, wherein the composition comprises the ECL-labeled component.
15. The composition of any of items 10-13, wherein the composition comprises the surfactant.
16. The composition of any one of items 10-15, wherein the composition comprises the ECL-labeled component and the surfactant.
17. The composition of any of items 10-16, wherein the surfactant comprises a polyethylene glycol (PEG).
18. The composition of any of items 10-17, wherein the surfactant comprises an alkyl ether-PEG.
19. The composition of any of items 12-18, wherein the composition consists essentially of said components.
20. The composition of item 19, wherein the composition consists of said components.
21. A composition comprising:
   (a) an electrochemiluminescence (ECL) co-reactant selected from N-tert-butyldiethanolamine (tBDEA), methyldiethanolamine (MDEA), 3-[Bis-(2-hydroxy-ethyl)-amino]-propane-1-sulfonic acid (DEA-PS), and combination thereof;
   (b) an ionic component; and
   (c) a surfactant;
   wherein the composition has a pH of about 7.0 to about 8.0.
22. A composition consisting essentially of:
   (a) an electrochemiluminescence (ECL) co-reactant selected from N-tert-butyldiethanolamine (tBDEA), methyldiethanolamine (MDEA), 3-[Bis-(2-hydroxy-ethyl)-amino]-propane-1-sulfonic acid (DEA-PS), and combination thereof; (b) an ionic component; and (c) a surfactant, or
   (a) an electrochemiluminescence (ECL) co-reactant selected from N-tert-butyldiethanolamine (tBDEA), methyldiethanolamine (MDEA), 3-[Bis-(2-hydroxy-ethyl)-amino]-propane-1-sulfonic acid (DEA-PS), and combination thereof; (b) an ionic component; (c) a surfactant, and (d) an ECL-labeled component,
   wherein the composition has a pH of about 7.0 to about 8.0.
23. A composition consisting of:
   (a) an electrochemiluminescence (ECL) co-reactant selected from N-tert-butyldiethanolamine (tBDEA), methyldiethanolamine (MDEA), 3-[Bis-(2-hydroxy-ethyl)-amino]-propane-1-sulfonic acid (DEA-PS), and combination thereof; (b) an ionic component; (c) a surfactant; and (d) a pH buffering component, or
   (a) an electrochemiluminescence (ECL) co-reactant selected from N-tert-butyldiethanolamine (tBDEA), methyldiethanolamine (MDEA), 3-[Bis-(2-hydroxy-ethyl)-amino]-propane-1-sulfonic acid (DEA-PS), and combination thereof; (b) an ionic component; (c) a surfactant; (d) a pH buffering component, and (e) an ECL-labeled component,
   wherein the composition has a pH of about 7.0 to about 8.0.
24. The composition of item 4, 7, or 21, further comprising an ECL-labeled component.
25. The composition of item 5, 8, or 22, wherein the composition consists essentially of components (a), (b), and (c).
26. The composition of item 5, 8, or 22, wherein the composition consists essentially of components (a), (b), (c), and (d).
27. The composition of item 6 or 9, wherein the composition consists of components (a), (b), and (c).
28. The composition of item 6 or 9, wherein the composition consists of components (a), (b), (c), and (d).
29. The composition of item 23, wherein the composition consists of components (a), (b), (c), and (d).
30. The composition of item 23, wherein the composition consists of components (a), (b), (c), (d), and (e).
31. The composition of any of items 1 to 3, 7 to 9, 10-12, or 24-28, wherein concentration of the TEA is about 1000 mM to about 6500 mM.
32. The composition of any of items 1 to 20, 24-28, or 31, wherein concentration of the TEA is about 1100 mM to about 3500 mM.
33. The composition of item 32, wherein concentration of the TEA is about 1200 mM to about 1600 mM.
34. The composition of any of items 21-26, 29, or 30, wherein concentration of the ECL co-reactant is about 50 mM to about 250 mM.
35. The composition of item 34, wherein concentration of the ECL co-reactant is about 100 mM to about 200 mM.
36. The composition of any of items 1 to 35, wherein the ionic component comprises chloride ion.
37. The composition of item 36, wherein the ionic component comprises NaCl, KCl, LiCl, or combination thereof.
38. The composition of item 37, wherein the ionic component comprises NaCl.
39. The composition of item 37, wherein the ionic component comprises KCl.
40. The composition of any of items 1 to 39, wherein concentration of the ionic component is about 500 mM to about 1500 mM.
41. The composition of item 40, wherein concentration of the ionic component is about 600 mM to about 1200 mM.
42. The composition of item 41, wherein concentration of the ionic component is about 700 mM to about 1000 mM.
43. The composition of item 42, wherein concentration of the ionic component is about 800 mM to about 900 mM.
44. The composition of item 1 or 4, further comprising a surfactant.
45. The composition of any of items 2, 3, 5, 6, or 10-43, wherein the surfactant is a non-ionic surfactant.
46. The composition of item 45, wherein the surfactant comprises a phenol ether.
47. The composition of item 45, wherein the surfactant is 2-[4-(2,4,4-trimethylpentan-2-yl)phenoxy]ethanol.
48. The composition of item 45, wherein the surfactant does not comprise a phenol ether.
49. The composition of item 48, wherein the surfactant is Poloxamer 407, block copolymer of poly(ethylene oxide) (PEO) and poly(propylene oxide) (PPO) PEO₁₈-PPO₇₂-PEO₁₈, block copolymer of poly(ethylene glycol) (PEG) and poly(propylene glycol) (PPG) PEG₅-PPG₆₈-PEG₅, PPO₂₆-PEO₅-PPO₂₆, ethylenediamine tetrakis(propoxylate-block-ethoxylate) tetrol, polyethylene glycol dodecyl ether, polyethylene glycol hexadecyl ether, polysorbate 20, 2,4,7,9-tetramethyl-d-decyne-4,7-diol ethoxylate, an alkyl ether-polyethylene glycol (PEG), or combination thereof.
50. The composition of item 49, wherein the surfactant is an alkyl ether-polyethylene glycol (PEG).
51. The composition of any of items 7 to 9 or 50, wherein the alkyl ether-PEG is PEG(10) tridecyl ether, PEG(12) tridecyl ether, PEG(18) tridecyl ether, or combination thereof.
52. The composition of item 51, wherein the alkyl ether PEG is PEG(18) tridecyl ether.
53. The composition of any of items 2, 3, 5, 6, or 10 to 52, wherein concentration of the surfactant is about 0.1 mM to about 10 mM.
54. The composition of item 53, wherein concentration of the surfactant is about 0.5 mM to about 8 mM.
55. The composition of item 54, wherein concentration of the surfactant is about 1 mM to about 5 mM.
56. The composition of any of items 7 to 9 or 50-52, wherein concentration of the alkyl ether-PEG is about 0.1 mM to about 10 mM.
57. The composition of item 56, wherein concentration of the alkyl ether-PEG is about 0.5 mM to about 8 mM.
58. The composition of item 57, wherein concentration of the alkyl ether-PEG is about 1 mM to about 5 mM.
59. The composition of any of items 1 to 58, wherein the pH is about 7.4 to about 7.9.
60. The composition of item 59, wherein the pH is about 7.5 to about 7.8.
61. The composition of any of items 1, 2, 4, 5, 7, 8, 10 to 19, 24 to 26, or 31 to 60, wherein the composition does not comprise any of phosphate, Tris, HEPES, glycylglycine, borate, acetate, and citrate.
62. The composition of any of items 1, 2, 4, 5, 7, 8, 10-19, 24-26, or 31-60, wherein the composition does not comprise an additional component having a pKa of about 7.0 to about 8.0.
63. The composition of item 21, wherein the composition further comprises a pH buffering component.
64. The composition of item 22, wherein the composition further consists essentially of a pH buffering component.
65. The composition of item 63 or 64, wherein the pH buffering component is phosphate, Tris, HEPES, glycylglycine, borate, acetate, citrate, or combination thereof.
66. The composition of item 65, wherein the pH buffering component is phosphate.
67. The composition of item 65, wherein the pH buffering component is Tris.
68. The composition of any of items 63-67, wherein concentration of the pH buffering component is about 100 mM to about 300 mM.
69. The composition of item 68, wherein concentration of the pH buffering component is about 150 mM to about 250 mM.
70. The composition of any one of items 1-3, 5, 6, 8-20, 22, 24, 26, 28, 30, or 31-69, wherein the ECL-labeled component comprises a detection reagent that comprises an ECL label; or wherein the ECL-labeled component comprises a binding partner of a detection reagent, wherein the binding partner comprises an ECL label.
71. The composition of item 70, wherein the ECL-labeled component is a detection reagent that comprises an ECL label.
72. The composition of item 70, wherein the ECL-labeled component and the detection reagent comprise complementary oligonucleotides.
73. The composition of any one of items 70-72, wherein the ECL label comprises an electrochemiluminescent organometallic complex.
74. The composition of item 73, wherein the electrochemiluminescent organometallic complex comprises ruthenium, osmium, iridium, or rhenium.
75. The composition of item 74, wherein the electrochemiluminescent organometallic complex comprises ruthenium.
76. The composition of item 75, wherein the electrochemiluminescent organometallic complex comprises a substituted or unsubstituted bipyridine or a substituted or unsubstituted phenanthroline.
77. The composition of item 76, wherein the electrochemiluminescent organometallic complex comprises at least one substituted bipyridine ligand, wherein the substituted bipyridine ligand comprises at least one sulfonate group.
78. The composition of item 77, wherein the electrochemiluminescent organometallic complex comprises at least two substituted bipyridine ligands, wherein each substituted bipyridine ligand comprises at least one sulfonate group.
79. The composition of item 77 or 78, wherein the substituted bipyridine ligand is a compound of Formula I:

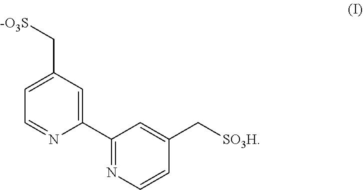

80. The composition of any one of items 70-79, wherein the ECL label is a compound of Formula II:

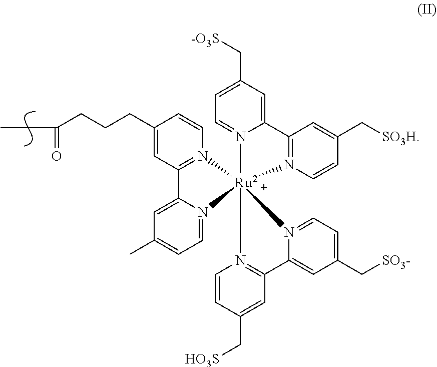

81. The composition of any one of items 1 to 80, wherein the composition is in dry form.
82. A method for generating electrochemiluminescence (ECL), comprising:
   (a) contacting an electrode with:
      (i) a TEA composition comprising TEA; an ionic component; and optionally a surfactant, or the composition of any of items 1-81; and
      (ii) an ECL label; and
   (b) applying a voltage to the electrode, thereby generating ECL.
83. The method of item 82, further comprising detecting the generated ECL.
84. The method of item 82 or 83, wherein the electrode is present on a surface.
85. The method of any of items 82-84, wherein the ECL label is present on an ECL-labeled component.
86. The method of any of items 82-84, wherein the ECL label is present in a sample.
87. The method of item 85, wherein step (a) further comprises contacting the electrode with a sample that comprises a binding partner of the ECL-labeled component, wherein the ECL-labeled component and the binding partner form a binding complex, and wherein the method comprises detecting the binding complex by detecting the generated ECL.
88. The method of item 85, wherein the ECL-labeled component is present in a binding complex, and wherein the method comprises detecting the binding complex by detecting the generated ECL.
89. The method of item 88, wherein the ECL-labeled component in the binding complex is a first copy of a detection reagent comprising the ECL label, and the binding complex comprises a binding reagent immobilized on the surface and the first copy of the detection reagent.
90. The method of item 88 or 89, further comprising forming the binding complex.
91. The method of item 90, wherein the binding complex is formed prior to or during step (a) of the method.
92. The method of any of items 89-91, wherein the binding complex is formed by incubating an assay mixture comprising the binding reagent, the first copy of the detection reagent, and a second copy of the detection reagent that comprises an ECL label, under conditions wherein
   the binding complex is formed on the surface, and the second copy of the detection reagent remains in solution.
93. The method of any of items 89-91, wherein the binding complex is formed by incubating an assay mixture comprising the binding reagent, the first copy of the detection reagent, a second copy of the detection reagent that comprises an ECL label, and the TEA composition or the composition of any of items 1-81, under conditions wherein
   the binding complex is formed on the surface, and the second copy of the detection reagent remains in solution.
94. The method of any of items 89-91, wherein the binding complex is formed by
   combining a sample with the first copy of the detection reagent, a second copy of the detection reagent that comprises an ECL label, and the TEA composition or the composition of any of items 1-81, thereby forming an assay mixture; and
   contacting the assay mixture with the binding reagent, under conditions wherein the binding complex is formed on the surface, and the second detection reagent remains in solution.
95. The method of item 85-94, wherein each of the sample, the TEA composition or the composition of any of items 1-81, and the ECL-labeled component is dry;
   wherein each of the sample, the TEA composition or the composition of any of items 1-81, and the ECL-labeled component is liquid; or
   wherein one or more of the sample, the TEA composition or the composition of any of items 1-81, and the ECL-labeled component is dry, and the remaining component(s) is liquid.
96. The method of item 95, wherein the TEA composition or the composition of any of items 1-81 is dry and present on the surface.
97. The method of any of items 88-96, wherein the binding complex further comprises an analyte, and wherein the method comprises detecting the analyte.
98. The method of any of items 82-97, further comprising measuring the generated ECL, thereby quantifying the amount of the ECL label, the ECL-labeled component, the binding complex, or the analyte.
99. A method of detecting a binding complex, comprising:
   (a) contacting a liquid sample with a surface comprising
      a TEA composition, wherein the TEA composition comprises TEA, an ionic component, and optionally a surfactant; or
      the composition of any one of items 1-81,
   wherein the liquid sample comprises an ECL-labeled component; or wherein the liquid sample comprises a binding partner of an ECL-labeled component, and the method further comprises contacting the surface with the ECL-labeled component,
   thereby forming a binding complex on the surface that comprises the ECL-labeled component;
   (b) applying a voltage to the surface to generate ECL; and
   (c) detecting the generated ECL, thereby detecting the binding complex.
100. A method of detecting a binding complex, comprising:
   (a) contacting a liquid sample with a surface comprising an ECL-labeled component and
      a TEA composition, wherein the TEA composition comprises TEA, an ionic component, and optionally a surfactant; or
      the composition of any one of items 1-81,
   wherein the liquid sample comprises a binding partner of an ECL-labeled component,
   thereby forming a binding complex on the surface that comprises the ECL-labeled component;
   (b) applying a voltage to the surface to generate ECL; and
   (c) detecting the generated ECL, thereby detecting the binding complex.
101. A method for detecting a binding complex, comprising:
   (a) forming a binding complex on a surface, wherein the surface optionally comprises an electrode, and wherein the binding complex comprises an ECL-labeled component;
   (b) contacting the binding complex with:
      a TEA composition comprising TEA, an ionic component, and optionally a surfactant; or
      the composition of any one of items 1-81;
   (c) applying a voltage to the surface to generate ECL; and
   (d) detecting the generated ECL, thereby detecting the binding complex.
102. A method for detecting an analyte of interest in a sample, comprising:
   (a) contacting the sample with: (i) a surface comprising a binding reagent, wherein the binding reagent specifically binds to the analyte; and (ii) a detection reagent that specifically binds to the analyte, wherein the detection reagent comprises an ECL label, thereby forming a binding complex on the surface comprising the binding reagent, the analyte, and the detection reagent;
   (b) contacting the binding complex on the surface with:
      a TEA composition comprising TEA, an ionic component, and optionally a surfactant; or the composition of any of items 1-81;
   (c) applying a voltage to the surface to generate ECL; and
   (d) detecting the generated ECL, thereby detecting the analyte.
103. The method of any one of items 99-102, wherein the method does not comprise a wash step.
104. The method of any one of items 99-102, wherein step (a) does not comprise a wash step.
105. The method of any one of items 99-101, wherein the ECL-labeled component comprises a detection reagent that comprises an ECL label, or wherein the ECL-labeled component comprises a binding partner of a detection reagent, wherein the binding partner comprises an ECL label.
106. The method of item 105, wherein the ECL-labeled component comprises a detection reagent, and wherein the binding complex comprises a binding reagent and the detection reagent.
107. The method of item 105, wherein the ECL-labeled component comprises a binding partner of a detection reagent, and wherein the binding complex comprises a binding reagent, the detection reagent, and the binding partner.
108. The method of item 107, wherein the detection reagent and the ECL-labeled component comprise complementary oligonucleotides.
109. A method for detecting a binding complex, comprising:
   (a) forming an assay mixture by combining a sample with:
      i. a TEA composition comprising TEA, an ionic component, and optionally a surfactant, or
         the composition of any of items 1-81; and
      ii. a detection mixture comprising at least two copies of a detection reagent, wherein each copy of the detection reagent comprises an ECL label;
   (b) contacting the assay mixture with a binding reagent immobilized on a surface, wherein the surface optionally comprises an electrode, under conditions wherein
      i. a binding complex is formed on the surface, the binding complex comprising the binding reagent and a first copy of the detection reagent; and
      ii. a second copy of the detection reagent remains in solution;
   (c) applying a voltage to the surface to generate ECL; and
   (d) detecting the generated ECL, thereby detecting the binding complex.
110. A method for detecting a binding complex, comprising:
   (a) incubating an assay mixture comprising:
      i. a binding reagent immobilized on a surface, wherein the surface optionally comprises an electrode; and
      ii. a detection mixture comprising at least two copies of a detection reagent,
         wherein each copy of the detection reagent comprises an ECL label; under conditions wherein
      i. a binding complex is formed on the surface, the binding complex comprising the binding reagent and a first copy of the detection reagent; and
      ii. a second copy of the detection reagent remains in solution;
   (b) contacting the binding complex with:
      a TEA composition comprising TEA, an ionic component, and optionally a surfactant; or
      the composition of any of items 1-81;
   (c) applying a voltage to the surface to generate ECL; and
   (d) detecting the generated ECL, thereby detecting the binding complex.
111. The method of item 109 or 110, wherein the second copy of the detection reagent is not removed prior to any of steps (b) to (d).
112. The method of item 109 or 110, wherein the second copy of the detection reagent is not removed prior to step (d).
113. A method for detecting a binding complex, comprising:
   (a) incubating an assay mixture comprising:
      i. a binding reagent immobilized on a surface, wherein the surface optionally comprises an electrode;
      ii. a detection mixture comprising at least two copies of a detection reagent, wherein each copy of the detection reagent comprises an ECL label; and
      iii. a TEA composition comprising TEA, an ionic component, and optionally a surfactant; or
         the composition of any of items 1-81;
   under conditions wherein
      i. a binding complex is formed on the surface, the binding complex comprising the binding reagent and a first copy of the detection reagent; and
      ii. a second copy of the detection reagent remains in solution;
   (b) applying a voltage to the surface to generate ECL; and
   (c) detecting the generated ECL, thereby detecting the binding complex.
114. The method of item 113, wherein the second copy of the detection reagent is not removed prior to any of steps (b) or (c).
115. The method of item 113, wherein the second copy of the detection reagent is not removed prior to step (c).
116. The method of any of items 106-115, wherein the binding complex further comprises an analyte, and the binding reagent and the first copy of the detection reagent each specifically binds to the analyte.
117. The method of any of items 109-116, wherein at least two copies of the binding reagent are immobilized on the surface, and wherein a first copy of the binding reagent forms a complex with the first copy of the detection reagent, and a second copy of the binding reagent binds to a competitor such that the second copy of the binding reagent does not form a complex with the second copy of the detection reagent.
118. The method of any of items 109-116, wherein at least two copies of the binding reagent are immobilized on the surface, and wherein a first copy of the binding reagent forms a complex with the first copy of the detection reagent, and the second copy of the detection reagent binds to a competitor such that the second copy of the binding reagent does not form a complex with the second copy of detection reagent.
119. The method of any of items 109-115, wherein the binding reagent binds to the first copy of the detection reagent to form the binding complex.
120. The method of any of items 99-119, wherein the method is a multiplexed method capable of simultaneously detecting one or more binding complexes.
121. The method of any of items 99-119, wherein the method is a multiplexed method capable of simultaneously detecting one or more analytes.
122. The method of any one of items 99-121, wherein the TEA composition or the composition of any of items 1-81 is in dry form.
123. The method of any one of items 109-122, wherein the detection mixture is in dry form.
124. The method of any one of items 109-123, wherein the TEA composition or the composition of any of items 1-81 and the detection mixture are in dry form.
125. The method of any of items 102-124, wherein the binding reagent and the detection reagent each comprises an antibody or antigen-binding fragment thereof, antigen, ligand, receptor, oligonucleotide, hapten, epitope, mimotope, or aptamer.
126. A method for quantifying the amount of an ECL label in a sample, comprising:
   (a) contacting an electrode with
      (i) a TEA composition comprising TEA, an ionic component, and optionally a surfactant, or
         the composition of any of items 1-81; and
      (ii) the sample comprising the ECL label;
   (b) applying a voltage to the electrode;
   (c) generating ECL;
   (d) measuring the ECL; and
   (e) quantifying the amount of the ECL label from the measured ECL.
127. A method for producing a composition, comprising combining:
   (a) triethanolamine (TEA);
   (b) an ionic component;
   (c) a surfactant; and
   (d) an ECL-labeled component,
   wherein the method does not comprise adding an additional pH buffering component.
128. The method of any of items 82-127, wherein the ECL label comprises an ECL-active organometallic complex.
129. The method of item 128, wherein the ECL-active organometallic complex comprises ruthenium.
130. The method of item 128 or 129, wherein the ECL-active organometallic complex comprises at least one substituted bipyridine ligand, wherein the substituted bipyridine ligand comprises at least one sulfonate group.
131. The method of item 130, wherein the ECL label is a compound of Formula II.
132. The method of any of items 84-131, wherein the surface comprises a well of a multi-well plate.
133. The method of any of items 84-131, wherein the surface comprises an assay cartridge.
134. The method of any of items 84-131, wherein the surface comprises a particle.
135. The method of any of items 84-134, wherein the surface comprises an electrode.
136. The method of item 134, further comprising collecting the particle on an electrode, and applying the voltage to the particle on the electrode.
137. The method of any of items 82-98, 135, or 136, wherein the electrode comprises a carbon electrode, a platinum electrode, a gold electrode, or a silver electrode.
138. The method of item 137, wherein the electrode is a carbon ink electrode.
139. An assay module comprising a TEA composition in dry form, wherein the TEA composition comprises TEA, an ionic component, and optionally a surfactant.
140. The assay module of item 139, wherein the assay module is a multi-well plate or an assay cartridge.
141. The assay module of item 139 or 140, wherein the assay module further comprises a binding reagent and/or a detection reagent in dry form.
142. The assay module of item 141, wherein the assay module further comprises the detection reagent.
143. The assay module of item 141 or 142, wherein the detection reagent comprises an ECL label.
144. The assay module of item 143, wherein the ECL label comprises an ECL-active organometallic complex.
145. The assay module of item 144, wherein the ECL-active organometallic complex comprises ruthenium.
146. The assay module of item 144 or 145, wherein the ECL-active organometallic complex comprises at least one substituted bipyridine ligand, wherein the substituted bipyridine ligand comprises at least one sulfonate group.
147. The assay module of item 146, wherein the ECL label is a compound of Formula II.
148. A kit comprising, in one or more containers, vials, or compartments:
   (a) a TEA composition comprising TEA, an ionic component, and optionally a surfactant; or
      the composition of any of items 1-81; and
   (b) optionally a surface comprising an electrode, wherein the TEA composition does not comprise an additional pH buffering component.
149. The kit of item 148, further comprising an assay instrument, an assay reagent, a calibration reagent, an ECL label, or combination thereof.
150. A kit comprising the composition of any of items 1 to 81 and:
   an assay instrument, an assay reagent, a calibration reagent, a surface, an ECL label, or combination thereof.
151. The kit of any of items 148-150, wherein one or more components of the kit is provided in dry form.
152. The kit of item 148, wherein the kit comprises a surface, and wherein the TEA composition is provided in dry form.
153. The kit of item 148, wherein the kit comprises a surface, and wherein the TEA composition is provided on the surface.
154. The kit of any of items 149-153, wherein the assay reagent comprises a binding reagent, a detection reagent, or both.
155. The kit of item 154, wherein the binding reagent and the detection reagent each comprises an antibody or antigen-binding fragment thereof, antigen, ligand, receptor, oligonucleotide, hapten, epitope, mimotope, or aptamer.
156. The kit of item 154 or 155, wherein the kit comprises a surface, and wherein the binding reagent and/or the detection reagent is provided on the surface.
157. The kit of item 154 or 155, wherein the kit comprises a surface and a reagent for immobilizing the binding reagent to the surface.
158. The kit of any of items 154-157, wherein the kit comprises a detection reagent that comprises an ECL label.
159. The kit of any of items 154-157, wherein the kit comprises a detection reagent and a reagent for conjugating the detection reagent to an ECL label.
160. The kit of any of items 150-159, wherein the ECL label comprises an ECL-active organometallic complex.
161. The kit of item 160, wherein the ECL-active organometallic complex comprises ruthenium.
162. The kit of item 160 or 161, wherein the ECL-active organometallic complex comprises at least one substituted bipyridine ligand, wherein the substituted bipyridine ligand comprises at least one sulfonate group.
163. The assay module of item 162, wherein the ECL label is a compound of Formula II.
164. The kit of any of items 148-163, wherein the kit comprises a surface and the surface comprises a well of a multi-well plate.
165. The kit of any of items 148-163, wherein the kit comprises a surface and the surface comprises an assay cartridge.
166. The kit of any of items 148-165, wherein the kit comprises a surface and the surface comprises a particle.
167. The composition, method or kit of any of the preceding items, wherein the presence of the binding complex is detected using an ECL-labeled oligonucleotide probe comprising an ECL label and a quenching moiety, wherein said quenching moiety is in proximity to said ECL label and quenches said ECL label at least when the ECL-labeled oligonucleotide probe is not hybridized to a complementary oligonucleitde, for example wherein the ECL-labeled oligonucleotide probe comprises a stem-loop or hairpin structure, wherein said quenching moiety is in proximity to said ECL label and quenches said ECL label when the oligonucleotide probe is in a stem-loop or hairpin configuration, but does not quench the ECL label when the stem-loop or hairpin structure is in an open configuration.
168. The composition, method or kit of item 167, wherein the ECL-labeled oligonucleotide probe comprises an oligonucleotide sequence that is complementary to an oligonucleotide sequence of a binding partner and/or binding complex.
169. The composition, method or kit of item 167, wherein the binding partner and/or binding complex comprises an analyte.
170. The composition, method or kit of item 169, wherein the analyte comprises a peptide.
171. The composition, method or kit of item 169, wherein the analyte comprises an oligonucleotide.
172. The composition, method or kit of item 171, wherein the analyte is the oligonucleotide of the binding partner.
173. The composition, method or kit of any one of items 169-171, wherein the analyte is labeled with the oligonucleotide.
174. The composition, method or kit of item 173, wherein the analyte is labeled with the oligonucleotide by binding the analyte with a detection reagent comprising the oligonucleotide.
175. The composition, method or kit of any one of items 168-174, wherein the oligonucleotide of the binding partner and/or binding complex comprises multiple copies of the sequence complementary to the oligonucleotide sequence of the ECL-labeled oligonucleotide probe.
176. The composition, method or kit of item 175, wherein the multiple copies of the sequence complementary to the oligonucleotide sequence of the ECL-labeled oligonucleotide probe are the product of an amplification reaction.
177. The composition, method or kit of item 173, wherein the analyte is labeled with the oligonucleotide by binding the analyte with a detection reagent comprising an oligonucleotide primer, and wherein the oligonucleotide primer is extended by a polymerase to generate the oligonucleotide that comprises multiple copies of the sequence complementary to the oligonucleotide sequence of the ECL-labeled oligonucleotide probe.
178. The composition, method or kit of items 176 or 177, wherein the amplification reaction is a rolling circle amplification reaction.
179. The composition, method or kit of any one of items 167-178, wherein the ECL-labeled oligonucleotide probe is the ECL-labeled component, the binding reagent and/or the detection reagent.
180. The composition, method or kit of any one of items 167-179, wherein the ECL-labeled oligonucleotide probe is selectively dequenched when hybridized to a complementary oligonucleotide.
181. The composition, method or kit of item 180, wherein the selective dequenching comprises hybridizing an ECL-labeled oligonucleotide having a molecular beacon-like design to a complementary oligonucleotide, such that the quenching moiety is no longer in proximity to the ECL label, or cleaving the quenching moiety only from probes which are hybridized to a complementary oligonucleotide thereby releasing the quenching moiety into solution, and leaving the ECL-labeled probe hybridized to the complementary oligonucleotide.
182. The composition, method or kit of item 181, wherein cleaving the quenching moiety is performed by an enzyme, for example, a restriction endonuclease, e.g. a nicking endonuclease, an RNaseH2, or a polymerase having 5′ exonuclease activity.

### EXAMPLES

### Example 1. Assessment of Zwitterion and Hydroxyethyl Amine ECL Coreactants

The following list of ECL coreactants were tested for their ECL generation and ability to discriminate between surface-bound and free (in solution) ECL labels in a solid-surface ECL assay: tributylamine (TBA), (dibutyl)aminoethanol (DBAE), (diethyl)aminoethanol (DEAE), triethanolamine (TEA), butyldiethanolamine (BDEA), propyldiethanolamine (PDEA), ethyldiethanolamine (EDEA), methyldiethanolamine (MDEA), tert-butyldiethanolamine (tBDEA), dibutylamine (DBA), butylethanolamine (BEA), diethanolamine (DEA), dibutylamine propylsulfonate (DBA-PS), dibutylamine butylsulfonate (DBA-BS), butylethanolamine propylsulfonate (BEA-PS), butylethanolamine butylsulfonate (BEA-BS), diethanolamine propylsulfonate (DEA-PS), and diethanolamine butylsulfonate (DEA-BS). Each ECL read buffer composition was prepared with 150 mM of a specified ECL coreactant, 200 mM phosphate, 850 mM NaCl, and either TRITON™ X-100 (“TX100”) or PEG(18) tridecyl ether (“PEG18 TDE”) and adjusted to pH 7.5.

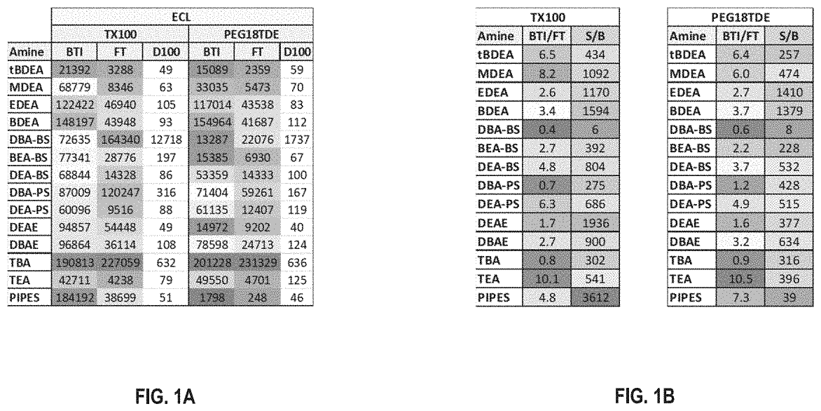

2 nM of IgG conjugated with biotin and an ECL label (“BTI”) was used as the control for bound label and contacted with an electrode surface coated with streptavidin. 500 mM of free ECL label (“FT”) was used as the control for free label. Results are shown in FIGS. 1A and 1B. FIG. 1A shows the ECL signal measured with BTI, FT, and background signal (“D100”) with ECL read buffer only (no label). FIG. 1B shows the ratio of ECL signal from bound label to ECL signal from free label (“BTI/FT”), and the signal-to-background ratio (“S/B”).

The raw values and ratios in FIGS. 1A and 1B present information regarding radical lifetimes, excited state formation efficiency for both oxidizing and reducing pathways, and ECL label-excited state reductive/oxidative quenching efficiency. The ECL signal sensitivity in TRITON™ X-100 supports a short-lived amine radical cation or low electron transfer efficiency to the ECL label in −1 oxidation state (Label-1).

From the results, it can be concluded that DBA-BS is sensitive to TRITON™ X-100, producing significantly more signal from free label than from bound label, which suggests a long-lived reducing radical was being produced leading to efficient reduction of the free ECL label. Moreover, the BTI/FT signal ratio of MDEA was higher than PIPES, an ECL coreactant known to have a short radical cation lifetime, and MDEA has a reasonable signal-to-background ratio but only in the presence of TRITON™ X-100, suggesting that MDEA also has a short radical cation lifetime. BDEA showed a strong signal from BTI and intermediate amount of signal from FT, suggesting a radical cation and reducing radical lifetimes in between those of TBA and TEA. Notably, TEA displayed a very low FT signal and reasonably high BTI signal, was insensitive to the presence or absence of TRITON™ X-100, and had the highest BTI/FT ratio of all the tested ECL coreactants.

### Example 2. Bound/Free Label Signal Ratios Vs. TEA Concentration

TEA has the ability to serve as both pH buffer and ECL coreactant due to its pKa of 7.7. Varying concentrations of TEA between 50 mM and 1600 mM were tested for their ECL generation properties. Each ECL read buffer composition tested contained TEA at a specified concentration and 850 mM NaCl, pH 7.8. The compositions were tested with BTI and FT labels in the same manner as for Example 1.

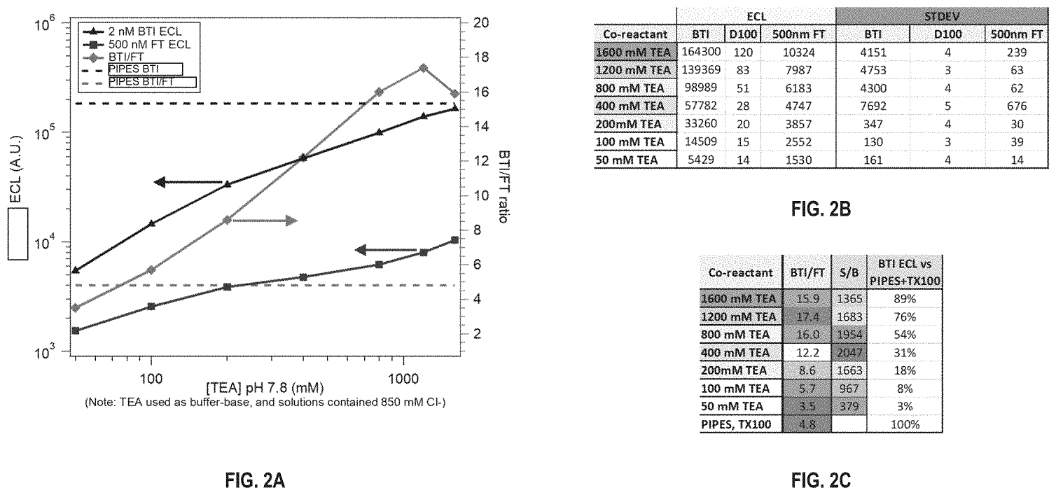

Results are shown in FIGS. 2A-2C. FIG. 2A shows a plot of the ECL generated from BTI and FT, and the BTI/FT ratio with varying concentration of TEA. The dashed lines at the top and bottom of the plot indicate the BTI signal and BTI/FT ratio, respectively, generated with a PIPES ECL read buffer. Thus, TEA shows a peak BTI/FT ratio at around 1200 mM concentration, and at TEA concentrations greater than 1200 mM, BTI signal was within 10 to 25% of PIPES ECL read buffer. The TEA radical cation and reducing radical lifetimes, and changes in buffer viscosity may have contributed to the general BTI/FT behavior and apparent decline at 1600 mM TEA. FIG. 2B shows the measured ECL signals from BTI, FT, and background (D100) with varying concentrations of TEA, and FIG. 2C shows the BTI/FT ratio, S/B ratio, and percent ECL generation compared with PIPES ECL read buffer.

### Example 3. ECL Signal Vs. Coreactant Concentration

The results of Example 2 suggested that increasing concentration of TEA produces higher ECL signal, which were contrary to the predictions based on the known behaviors of other ECL coreactants, such as PIPES. The ECL signal generated with a PIPES ECL read buffer and TEA ECL read buffer were measured at varying concentrations of the coreactants. The PIPES compositions contained 20 mM, 40 mM, or 80 mM PIPES, >0.1% TRITON™ X-100, and 80 to 320 mM potassium phosphate buffer pH 7.5. The TEA compositions contained 50 mM, 100 mM, or 200 mM TEA, 850 mM NaCl, and 1 mM PEG18 TDE. The compositions were tested with BTI as described for the previous Examples.

Results are shown in FIGS. 3A (ECL signal vs. PIPES concentration) and 3B (relative ECL signal vs. PIPES concentration and TEA concentration plotted together). FIG. 3A confirms that for PIPES ECL read buffer increasing PIPES concentration decreased the ECL signal. FIG. 3B shows the unexpected contrasting behavior of TEA, which showed a strong increase in ECL signal with increasing TEA concentration despite having short radical lifetimes.

### Example 4. Different Assay Formats

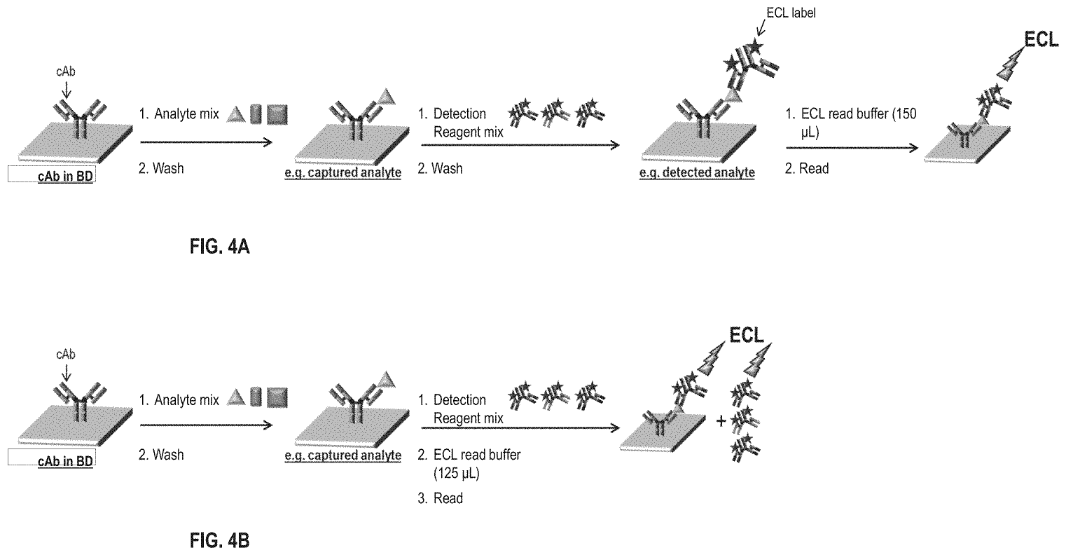

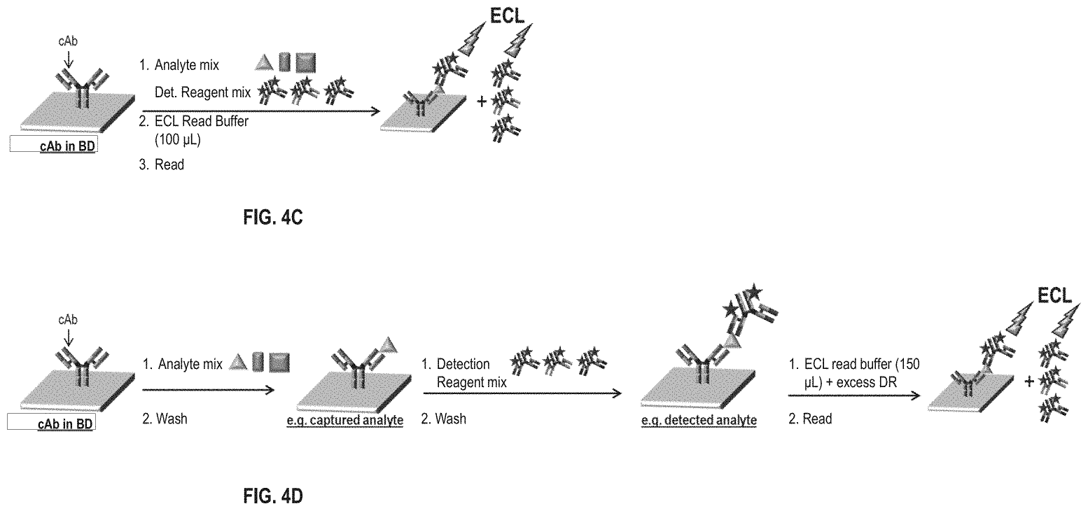

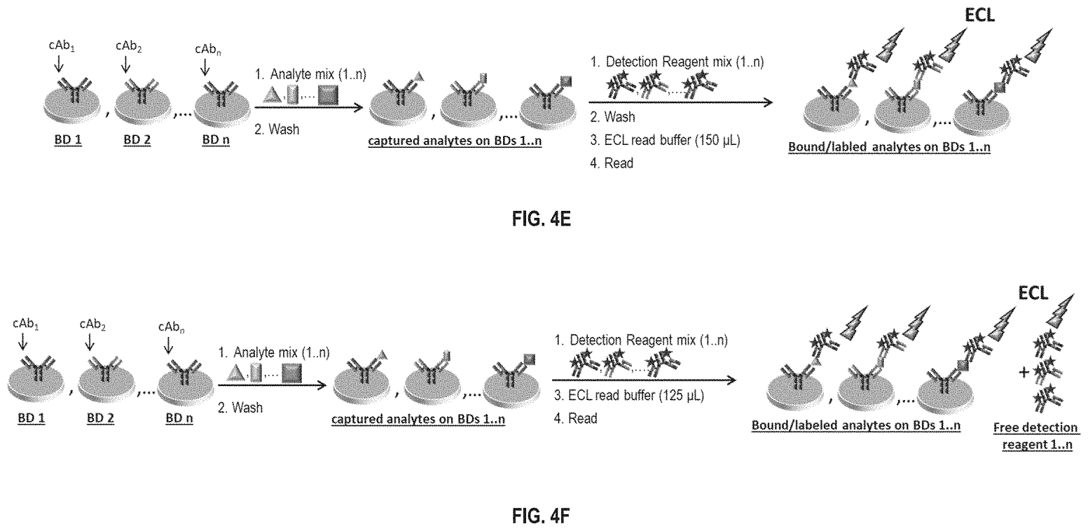

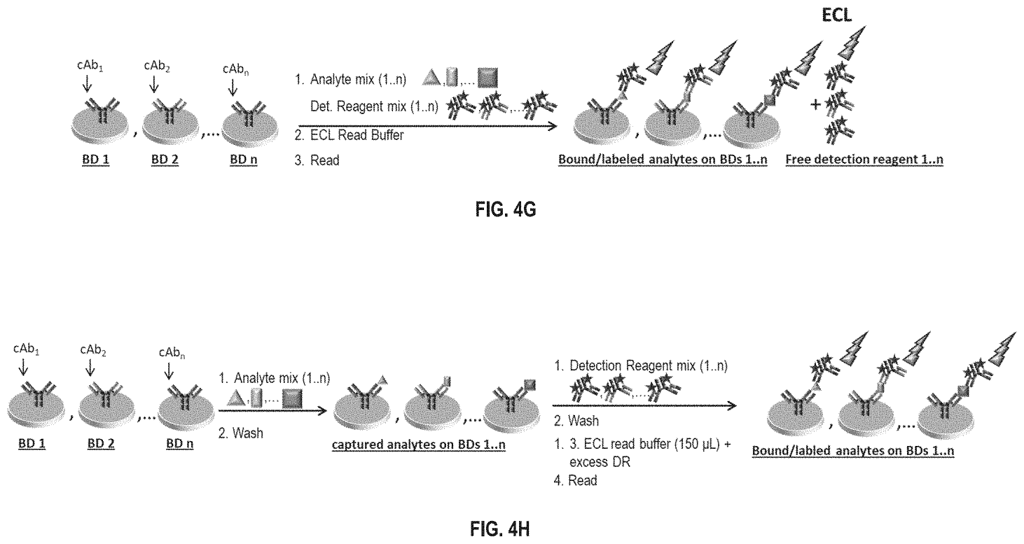

FIGS. 4A-4D illustrate four different assay formats, tested with ECL read buffers containing different ECL coreactants: TPA, BDEA, PIPES, and 1.2 M TEA. The assays were assessed with a panel of analytes. FIGS. 4E-4H illustrate multiplexed versions of the assays in FIGS. 4A-4D.

FIG. 4A illustrates a “standard” 2-step washed assay, wherein a capture antibody (“cAb”; binding reagent) immobilized on a binding domain (“BD”) on a surface is contacted with a mixture of analytes, one of which binds specifically to the capture antibody, and the surface is then washed, resulting in the analyte captured on the surface. A mixture of detection antibodies (“dAb”; detection reagent), each containing an ECL label and one of which binds specifically to the analyte, is then added to the surface, and the surface is then washed, resulting in a binding complex comprising the cAb, analyte, and dAb. ECL read buffer is then added to the surface, and the generated ECL is then read by an ECL reader instrument. FIG. 4E illustrates a multiplexed version of the “standard” 2-step washed assay, wherein one or more surfaces comprises a plurality of binding domains, each binding domain comprising a capture antibody that can bind to an analyte in the analyte mix. The surface(s) comprising the binding domains is washed after adding the analyte mix, resulting in a plurality of analytes captured on the binding domains. A mixture of detection antibodies, each containing an ECL label and can bind to an analyte in the analyte mix, is then added to the surface(s), and the surface is then washed, resulting in a plurality of binding complexes, each binding complex comprising a cAb, analyte, and dAb. ECL read buffer is then added to the surface, and the generated ECL is read by an ECL instrument.

In the Examples herein using the standard 2-step assay format, 50 μL of an analyte mix was added to plates, shaken for 2 hours at 705 rpm and room temperature. The plates were washed once with wash buffer, and 25 μL of the detection antibody mix was added to the plates, shaken for 1.5 hours at 705 rpm and room temperature. The plates were washed once with wash buffer, and 150 μL of ECL read buffer was added to the plates. The plates were then read with an ECL reader instrument.

FIG. 4B illustrates a “1-step” assay, wherein a capture antibody on a binding domain on a surface is contacted with an analyte mix, and the surface is then washed as in FIG. 4A. The detection antibody mix is then added, followed by the ECL read buffer without washing in-between adding the detection antibody mix and the ECL read buffer. The generated ECL is then read by an ECL reader instrument. FIG. 4F illustrates a multiplexed version of the “1-step” assay, wherein one or more surfaces comprises a plurality of binding domains, each binding domain comprising a capture antibody that can bind to an analyte in the analyte mix. The surface(s) comprising the binding domains is washed after adding the analyte mix as in FIG. 4E. The detection antibody mix is added to form a plurality of binding complexes, and ECL read buffer is then added without washing in between adding the detection antibody mix and the ECL read buffer. The generated ECL is then read by an ECL reader instrument.

In the Examples herein using the 1-step assay format, 50 μL of an analyte mix was added to plates, shaken for 2 hours at 705 rpm and room temperature. The plates were washed once with wash buffer, and 25 μL of the detection antibody mix was added to the plates, shaken for 1.5 hours at 705 rpm and room temperature. 125 μL of ECL read buffer was added to the plates. The plates were then read with an ECL reader instrument.

FIG. 4C illustrates a “1-step non-wash” assay, wherein a capture antibody on a binding domain on a surface is contacted with: an analyte mix and detection antibody mix, followed by the ECL read buffer without washing in between any of the steps. The generated ECL is then read by an ECL reader instrument. FIG. 4G illustrates a multiplexed version of the “1-step non-wash” assay, wherein one or more surfaces comprises a plurality of binding domains, each binding domain comprising a capture antibody that can bind to an analyte in the analyte mix. The surface(s) comprising the binding domains is contacted with an analyte mix and detection antibody mix to form a plurality of binding complexes, then ECL read buffer is added without washing in between any of the steps. The generated ECL is then read by an ECL reader instrument.

In the Examples herein using the 1-step non-wash assay format, 25 μL of an analyte mix was added to plates, followed by 25 μL of the detection antibody mix, then shaken for 2 hours at 705 rpm and room temperature. 100 μL of ECL read buffer was added to the plates. The plates were then read with an ECL reader instrument.

FIG. 4D illustrates a “mock ECL label” assay, wherein a capture antibody on a surface is contacted with an analyte mix, the surface is washed, a detection antibody mix is added, and the surface is optionally washed again, resulting in a binding complex as in FIG. 4A. The ECL read buffer is then added to the surface along with a detection antibody that comprises an ECL label and that does not bind to any component of the binding complex on the surface, which serves as a proxy for “free” ECL label in solution. The generated ECL is then read by an ECL reader instrument. FIG. 4H illustrates a multiplexed version of the “mock ECL label” assay, wherein one or more surfaces comprises a plurality of binding domains, each binding domain comprising a capture antibody that can bind to an analyte in the analyte mix. The surface(s) comprising the binding reagents is contacted with an analyte mix, the surface is washed, a detection antibody mix is added, and the surface is optionally washed again, resulting in a plurality of binding complexes as in FIG. 4E. The ECL read buffer is then added to the surface along with a detection antibody that comprises an ECL label and that does not bind to any component of the binding complex on the surface, which serves as a proxy for “free” ECL label in solution. The generated ECL is then read by an ECL reader instrument.

In the Examples herein using the mock ECL label assay format, 50 μL of an analyte mix was added to plates, shaken for 2 hours at 705 rpm and room temperature. The plates were washed once with wash buffer, and 25 μL of the detection antibody mix was added to the plates, shaken for 1.5 hours at 705 rpm and room temperature. The plates were washed once with wash buffer, and 150 μL of ECL read buffer containing excess detection reagent was added to the plates. The plates were then read with an ECL reader instrument.

### Example 5A. Assessment of ECL Read Buffers in Different Assay Formats

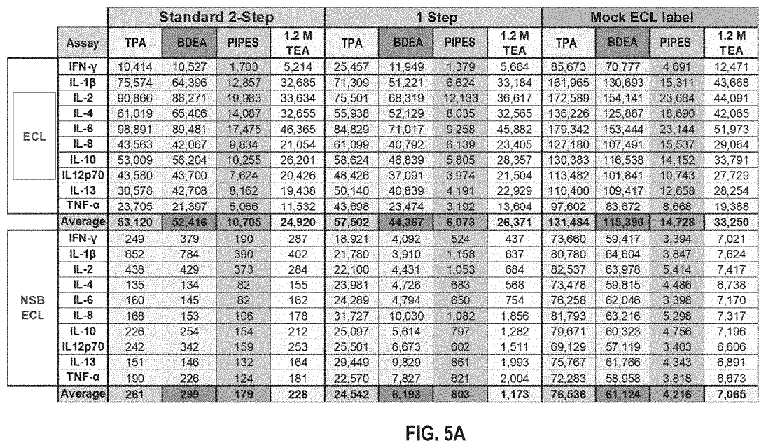

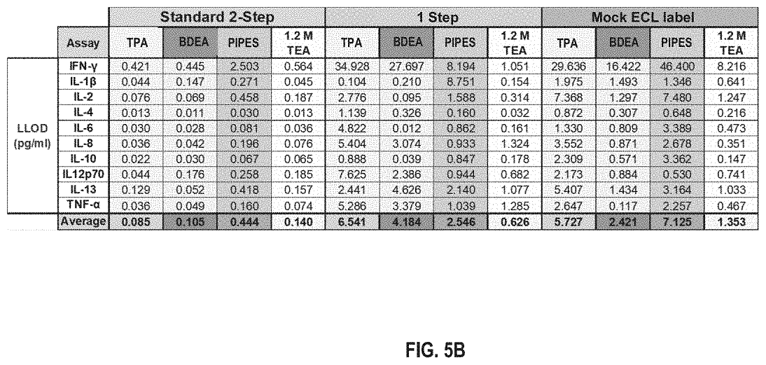

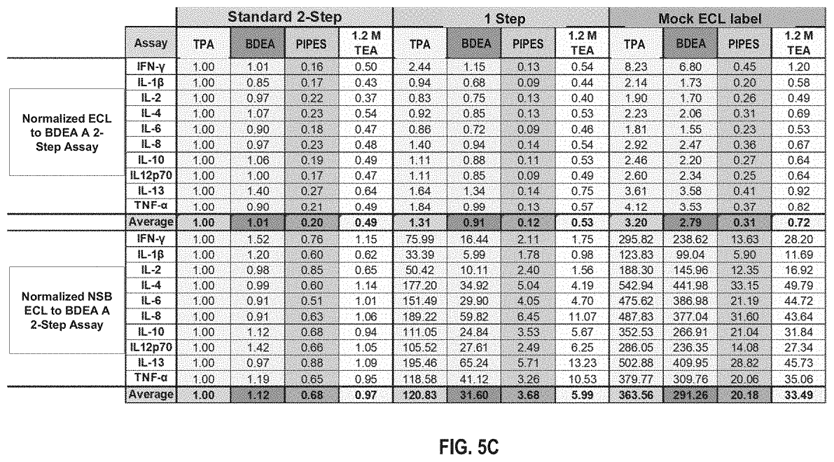

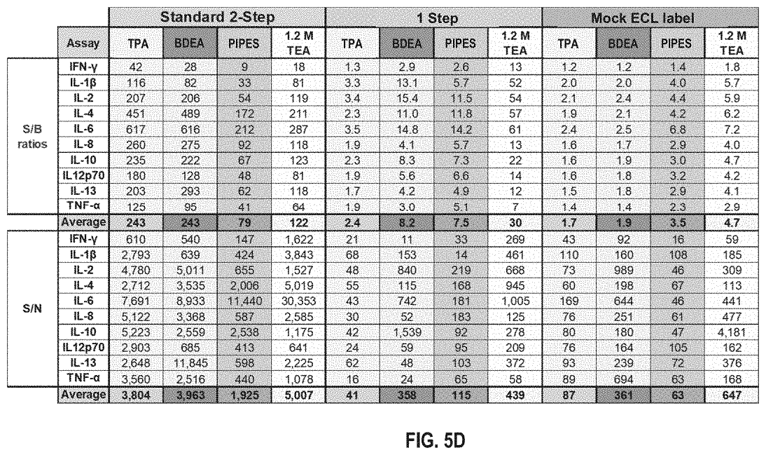

In Example 5A, the standard 2-step, 1-step, and mock ECL label multiplexed assay formats (shown in FIGS. 4E, 4F, and 4H) were tested. Read buffer was diluted ⅚^{th} the 1-step assay. Results of the specific ECL signals and non-specific binding (NSB) for these assays are shown in FIG. 5A, and the lowest limits of detection (LLOD) are shown in FIG. 5B. The specific ECL signal from TEA read buffer did not change significantly in a 1-step assay as compared with the standard 2-step assay. TEA had significantly improved performance over PIPES read buffer in the 1-step assay, due to increased specific signal and decreased NSB. Some elevation in background was observed across all ECL read buffers in the “mock ECL-label” assay, due to the high concentration of free detection antibody-ECL label in solution. TEA read buffer showed the best performance among the buffers in this assay format because of its excellent discrimination of bound vs. free label. Specific ECL signal for bound label from TEA read buffer was generally within 2× of the BDEA read buffer formulations, and thus the improved surface selectivity came with only minimal cost in overall signal generation.

In the standard 2-step assay, the LLOD of TEA read buffer was within 2- to 3-fold of the LLOD of a commercially available tripropylamine (TPA) read buffer. The ordering of average LLOD for the 1-step assay across different read buffers was as follows: TPA>BDEA>PIPES>TEA, with TEA providing the best (lowest) LLOD.

FIG. 5C shows a relative comparison of the signal in the presence of analyte (ECL) and in the absence of analyte (NSB) presented in FIG. 5A, with all ECL read buffers and assay formats normalized to the results obtained with a commercial TPA formulation in the standard 2-step assay format. In general, PIPES read buffer showed lower ECL signal than TEA read buffer across all assay formats, which is the inverse of the ECL generation efficiency data, suggesting that PIPES performance is possibly negatively affected by the immobilization of antibodies on the electrode surface or by exposure of the electrode to the sample matrices or diluents used during the assay. TEA and PIPES showed the lowest relative change in NSB signal between the standard 2-step and 1-step assay formats.

FIG. 5D shows the comparison of signal to background (S/B) and signal to noise (S/N) ratio across all ECL read buffers and assay formats. On average, TEA showed the smallest change in S/B between standard 2-step and 1-step assay formats. The average S/N ratios changed the least for TEA read buffer between all three assay formats. Thus, TEA read buffer demonstrated significant potential for use in non-wash assay formats.

### Example 5B. Assessment of ECL Read Buffers in Different Assay Formats

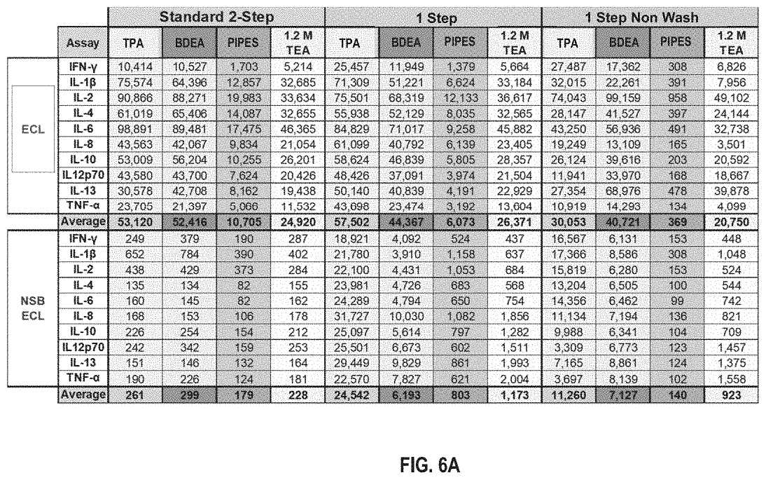

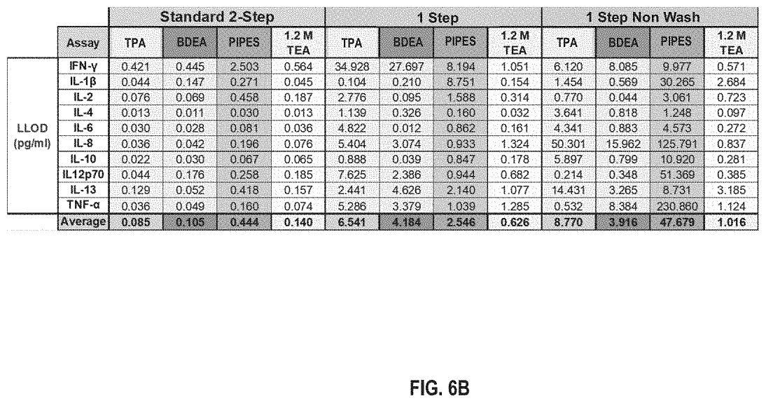

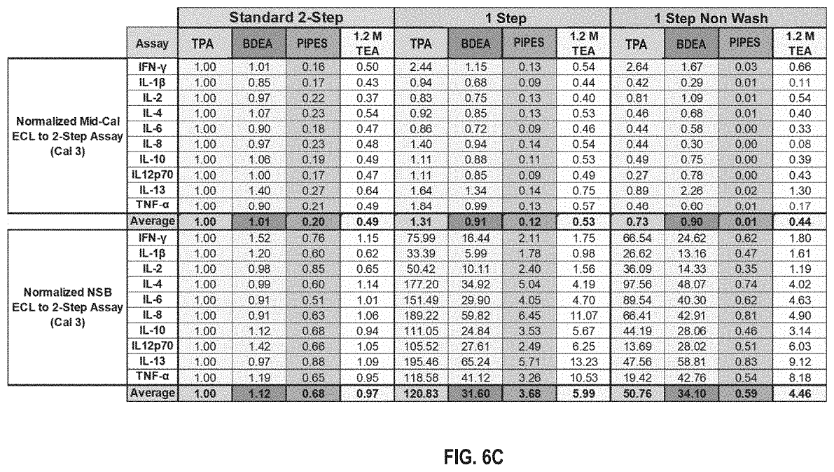

In Example 5B, the standard 2-step, 1-step, and 1-step non-wash multiplexed assay formats (shown in FIGS. 4E, 4F, and 4G) were tested. Read buffer was diluted ⅚^{th} in the 1-step assay, and ⅔^{rd} in the 1-step non-wash assay. Results of the specific ECL and NSB for these assays are shown in FIG. 6A, and the LLOD are shown in FIG. 6B. The specific ECL signal from TEA read buffer did not change significantly in the 1-step non-wash assay across most analytes, despite the ⅔^{rd} buffer dilution in the 1-step non-wash assay. There was also little to no NSB change for TEA read buffer in the 1-step non-wash assay vs. 1-step assay, likely due to the ⅔^{rd} dilution of the TEA read buffer, which causes a ˜30% decrease in ECL generation efficiency. PIPES read buffer performed significantly worse than TEA read buffer in the 1-step assay (as also observed in Example 4A) due to decreased specific ECL signal and increased NSB signal. In general, TEA read buffer LLOD was within 5× of TPA read buffer and BDEA read buffers in the standard 2-step assay format.

FIG. 6C shows a relative comparison of ECL and NSB results from FIG. 6A, with all ECL read buffers and assay formats normalized to the results obtained with TPA formulation in the standard 2-step assay format. On average, TEA read buffer showed little change in specific ECL and NSB between the 1-step assay and 1-step non-wash assay (with the exceptions being the analytes IL-1β, IL-8, and TNF-α, which showed a similar signal loss with the other ECL read buffers, suggesting that the issue is not related to the ECL coreactant but possibly with the 1-step analyte capture and/or binding complex formation steps). The poor performance of PIPES read buffer in the 1-step and 1-step non-wash assay formats is likely due to dilution of TRITON™ X-100 and possibly sensitivity of ECL generation in the presence of PIPES to effects of the assay conditions and the condition of the electrode surface.

### Example 6. Evaluation of TEA Read Buffer with Common Sample and Diluent Matrices

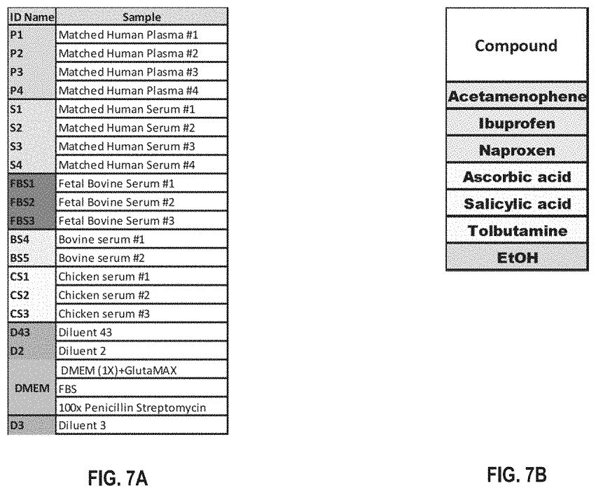

TEA read buffer ECL generation and background were tested with different sample matrices and metabolite and/or drug interferents. The TEA read buffer composition included 1.2 M TEA and 850 mM NaCl at pH 7.8. Surfaces were contacted with 2 nM BTI. The assays were conducted as shown in FIG. 4C (1-step non-wash assay), with the sample matrices (with or without interferents) added just prior to adding the ECL read buffer. FIG. 7A shows the sample matrices, and FIG. 7B shows the interferents tested.

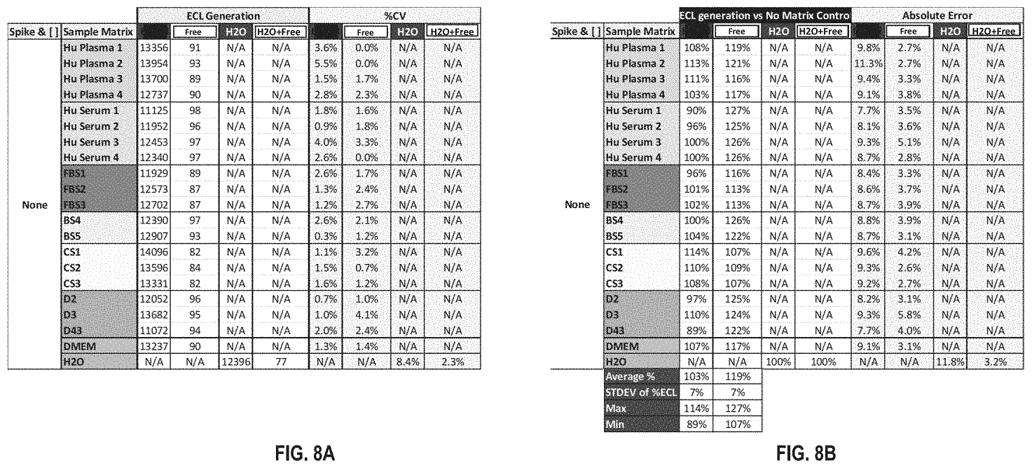

FIG. 8A shows the results of ECL signal generated from TEA read buffer with bound (“Bound”) and free ECL label (“Free”), with different sample matrices. “H2O” indicates signal from a control with water instead of sample matrix added to a well before TEA read buffer. The column headers with “Free” indicates 6 nM of free ECL label in mock diluent. FIG. 8B shows the results of FIG. 8A normalized to ECL signal generated from an assay in which sample matrices were not added. The results indicate that all tested sample matrices (e.g., human, animal, and proteinaceous) minimally influenced ECL generation efficiency of TEA read buffer from bound ECL label when performed in 1-step non-wash assays, with an average signal change of less than 5%. The 6 nM of free ECL label in diluent was not detectable with the TEA read buffer, and the slight background signal increase appeared to be matrix dependent.

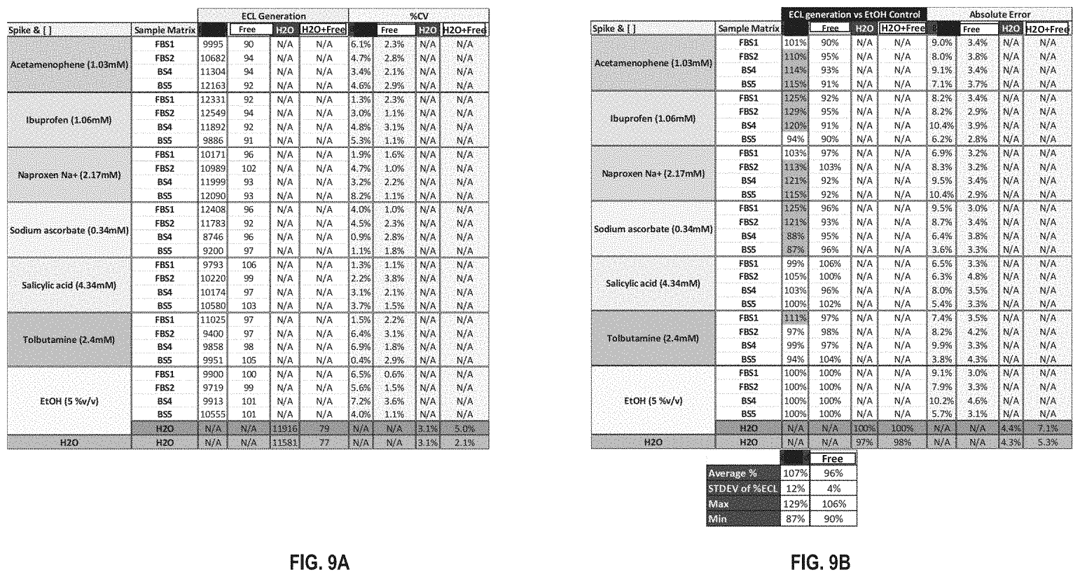

The sample matrices were then spiked with interferents (shown in FIG. 7B) at levels in excess of those commonly reported in human blood samples (see Lorenz et al., Diabetes Technology & Therapeutics 20(5):344-352 (2018)) and tested in the same manner as described above. FIG. 9A shows the results of ECL signal generated from TEA read buffer with bound and free ECL label with different interferents in different sample matrices. FIG. 9B shows the results of FIG. 9A normalized to ECL signal generated from an assay in which sample matrices and interferents were not added. The results indicate that the interferents in spiked FBS or BS showed minimal influence on the ECL generation efficiency of TEA read buffer from bound or free ECL label when performed in 1-step non-wash assays. The 6 nM free ECL label was barely detectable across all assays.

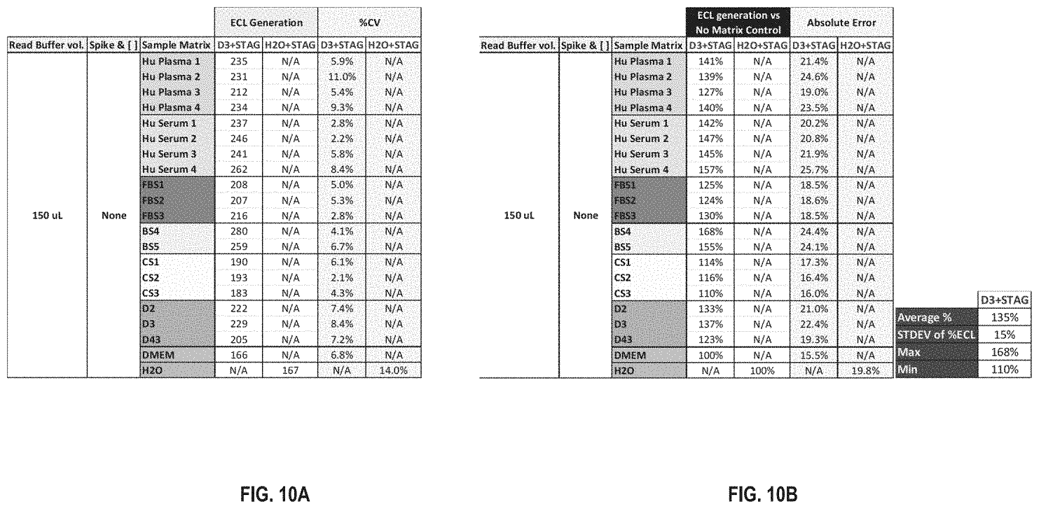

The sample matrix influence was tested on ECL generation from free ECL label at a higher concentration of 240 nM. FIG. 10A shows the results of ECL signal generated from TEA read buffer with free ECL label (“D3+STAG”) in different sample matrices. FIG. 10B shows the results of FIG. 10A normalized to ECL signal generated from an assay in which sample matrices were not added. The sample matrices showed low influence on ECL generation efficiency of TEA read buffer from free ECL label when performed in 1-step non-wash assays, with an average change of less than 35%. The ECL signals from DMEM culture media and control free ECL label were lower than the human/animal serum or plasmas, which generated ˜15 extra background counts. The higher signal for the proteinaceous human/animal serum or plasmas compared to the control signal was possibly due electrostatic attraction of ECL label to electrode adsorped proteins. The lower ECL signal from DMEM could have been possibly due to phenol red dye interference. The results further confirm that human, animal, diluent, and culture matrices minimally influence TEA read buffer ECL generation efficiency in 1-step non-wash assays.

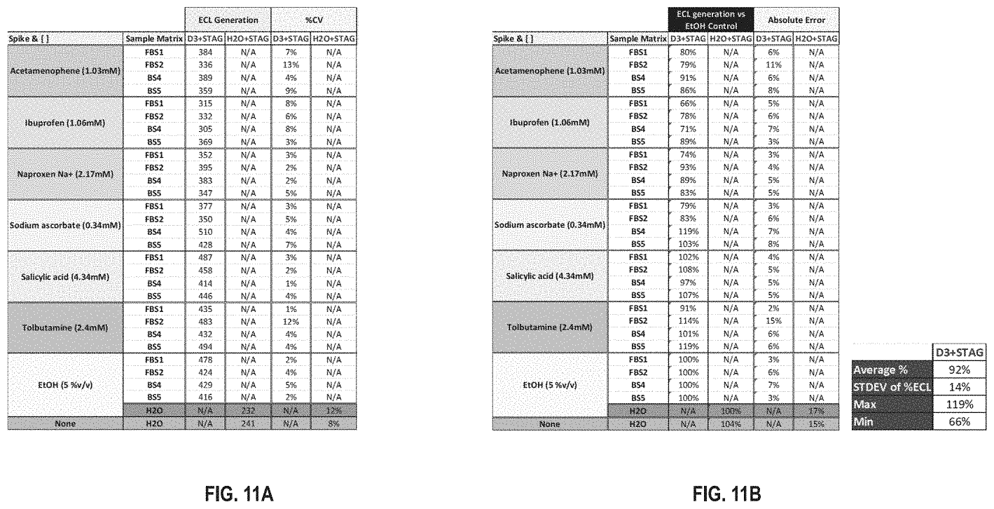

The higher concentration of free ECL label (240 nM) was tested with interferent-spiked sample matrices. FIG. 11A shows results of ECL signal generated from TEA read buffer with free ECL label with different interferents in different sample matrices. FIG. 11B shows the results of FIG. 11A normalized to ECL signal generated from an assay in which sample matrices and interferents were not added. The signal generation from free label using TEA read buffer remained low and consistent in the presence of the different interferents, when performed in 1-step non-wash assays. A small elevation in signal was observed in the interferent conditions relative to the control condition (“H2O” condition with no matrix and no interferent spike), which was an effect of the ethanol which was added to the matrix as the solvent for the interferents and not due to the interferents themselves.

### Example 7. Combinatorial ECL Coreactant Measurements

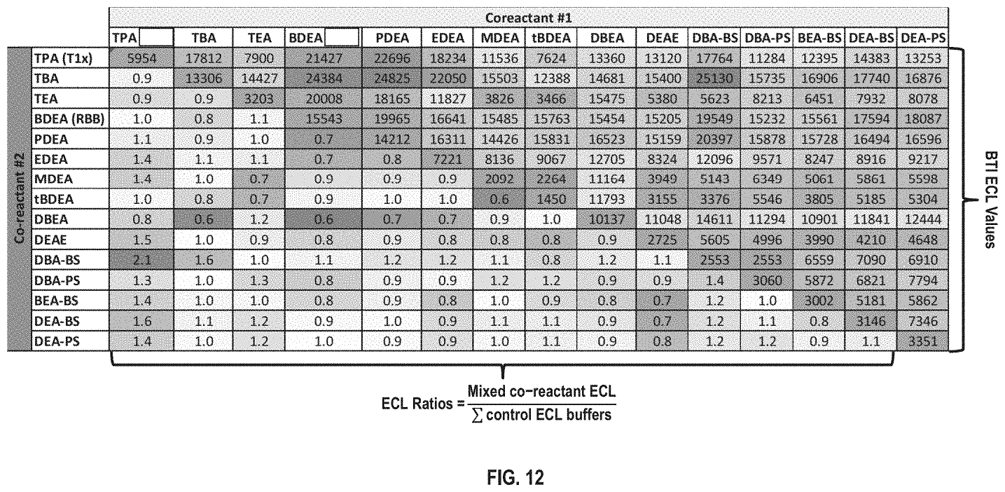

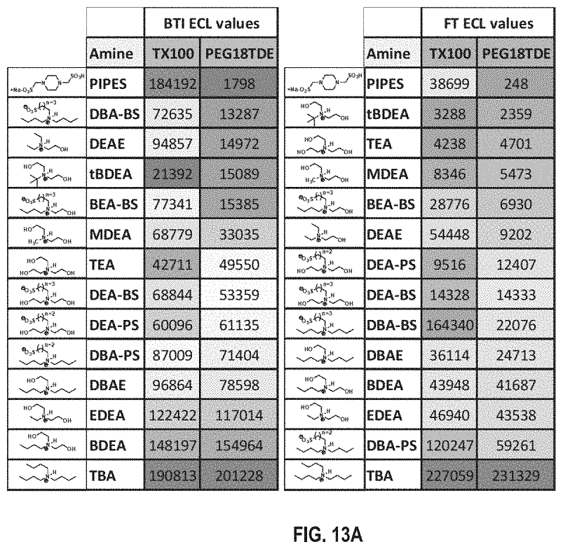

Combinations of ECL coreactants described in Example 1 were tested with a total concentration of 150 mM coreactant in 200 mM Tris, 50 mM KCl, 850 mM NaCl, 0.1% TRITON™ X-100, pH 7.8. Assays were performed with bound ECL label (BTI) as described in Example 1. Results are shown in FIG. 12. The top-right side of the chart in FIG. 12 shows the ECL signal generated from BTI, while the bottom-left side of the chart in FIG. 12 shows the ECL signal ratio of the mixed ECL coreactants to the sum of signal generated by the individual ECL coreactants. As shown in FIG. 12, combinations of TPA with other ECL coreactants showed possible non-linear effects.

### Example 8. Sensitivity of ECL Coreactants to TRITON™ X-100 Presence

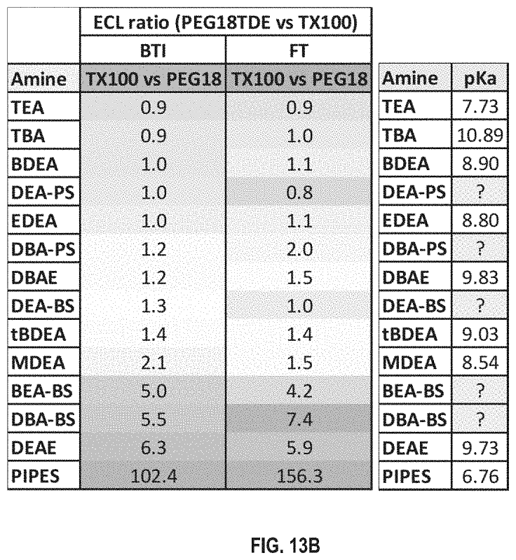

The ECL coreactants described in Example 1 were tested for sensitivity to the presence of TRITON™ X-100, which is required by the commonly-used ECL coreactant tripropylamine (TPA). Assays were performed with bound (BTI) and free (FT) ECL labels as described in Example 1. Results are shown in FIGS. 13A and 13B. FIG. 13A shows the ECL signal from BTI and FT for each ECL reactant in TRITON™ X-100 (TX100) and PEG(18) tridecyl ether (PEG18TDE), a non-electroactive surfactant. FIG. 13B shows the ratio of ECL generated in TRITON™ X-100 vs. PEG(18) tridecyl ether.

The compounds that were highly sensitive to TRITON™ X-100 were believed to have short radical cation lifetimes, and lower ECL signals were possibly due to poor electron transfer between the ECL label and coreactant, and/or rapid side reactions of ECL label/coreactant intermediates. Based on the results in FIGS. 13A and 13B, the ECL coreactants most sensitive to TRITON™ X-100 were: PIPES>>DEAE˜=DBA-BS˜=BEA-BS.

### Examples 9. Preparation of ECL-Labeled Oligonucleotide Molecular-Beacon-Like Probes

Exemplary ECL-labeled oligonucleotide molecular-beacon-like probes (MB) were prepared as follows. A general MB was designed based on an example from the literature (Mhlanga & Malmberg, Methods, 25(4):463-712001). A target sequence complementary to the loop of the MB was designed and inserted in a randomly generated oligo to form a 60-mer. The random 60-mer was selected from a pool of randomly generated sequences as the one with the lowest self-complementarity and therefore the least likely for form secondary structures that could possibly interfere with binding of the MB. A target sequence longer than the sequence complementary to the MB was used as this would be a better representation of a real biological assay where the sequence of interest is likely a region of a larger oligo to which the MB hybridize.

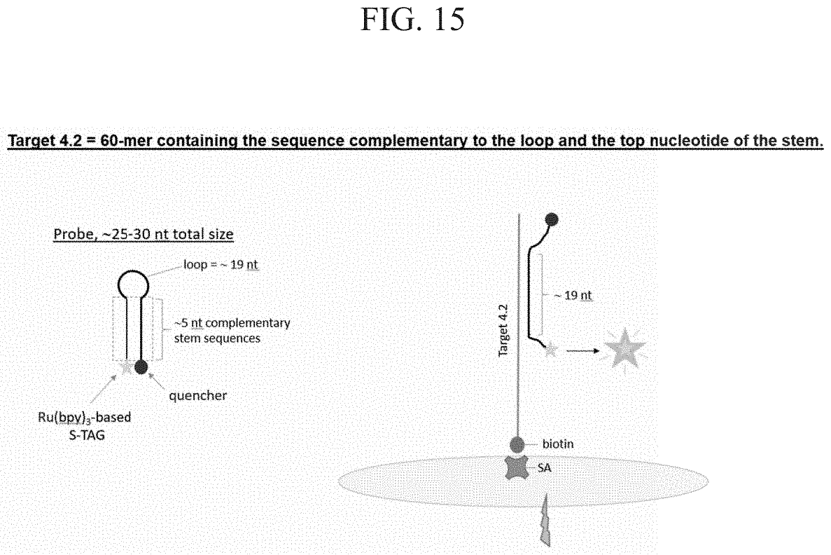

Two different Target sequences were designed, one 60-mer containing the sequence complementary to the loop of the molecular beacon (T4.1), and one 60-mer containing the sequence complementary to the loop and the top nucleotide of the stem (T4.2; FIG. 15). The T4.2 sequence was expected to bind the MBs tighter and destabilize the stem. The target oligonucleotides were biotin labeled to allow for immobilization on streptavidin coated plates.

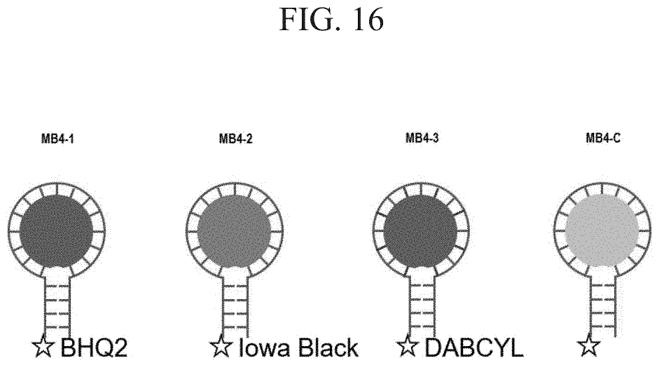

The quenching efficiency of three different quenchers on S-TAG ECL signal was evaluated. Four molecular beacons with 3′ quenchers and 5′ amines were ordered from IDT and labeled with S-TAG-NHS (Table 1, FIG. 16). The 3′ end was labeled with the following quenchers: Black Hole Quencher 2 (MB4-1), Iowa Black (MB4-2), Dabcyl (MB4-3). As a control MB4-C was not labeled with a quencher. The S-TAG labeled beacons were successfully purified from oligo production impurities, hydrolyzed S-TAG-NHS, and additional S-TAG species using anion exchange chromatography.

TABLE 1
Sequence of the ECL-labeled Oligonucleotide
Probes and Target Sequences

| | | | |
|---|---|---|---|
| ECL | MB4.1 | /5AmMC6/CCAAGCGAGCCCCCCA | SEQ ID |
| Probes | | TATTGTAGCTTGG/3BHQ_2/ | NO: 1 |
| | MB4.2 | /5AmMC6/CCAAGCGAGCCCCCCA | SEQ ID |
| | | TATTGTAGCTTGG/3IAbRQSp/ | NO: 2 |
| | MB4.3 | /5AmMC6/CCAAGCGAGCCCCCCA | SEQ ID |
| | | TATTGTAGCTTGG/3Dab/ | NO: 3 |
| | MB4.C | /5AmMC6/CCAAGCGAGCCCCCCA | SEQ ID |
| | | TATTGTAGCTTGG/ | NO: 4 |
| Target | T4.1 | /5Biosg/AAAGATGATAAGCTC | SEQ ID |
| Sequences | | CGGCAAGCAATATTGTACAATA | NO: 5 |
| | | TGGGGGGCTCCGATATAAACAGA | |
| | T4.2 | /5Biosg/AAAGATGATAAGCTC | SEQ ID |
| | | CGGCAAGCAATATTCTACAATA | NO: 6 |
| | | TOGGGGGCTCGGATATAAACAGA | |

FIG. 15 is an illustration of the experiments carried on in Examples 10-12. The target oligonucleotide (Target 4.2) is immobilized to the surface of an electrode (grey oval) by a biotin-streptavin interaction. In some of the experiments the surface of the electrode is uncoated, and the target oligonucleotide remains in solution. The ECL-labeled molecular beacon probes are permitted to hybridize to the target, separating the quencher (if present) from the ECL-label, dequenching the ECL label, permitting the detection of the ECL signal upon application of a voltage in the presence of an ECL co-reactant (not shown).

### Example 10. Performance of ECL-Labeled Oligonucleotide Molecular-Beacon-Like Probes

Initial experiments suggested that the MBs of Example 9 are more stable than anticipated, have higher Tm, and the best performance was observed for the T4.2 target sequence which was designed to slightly destabilize the stem. A temperature boost during the immobilization of the MB also increased the assay signal by melting the MB before allowing it to hybridize to the immobilized target sequence (data not shown).

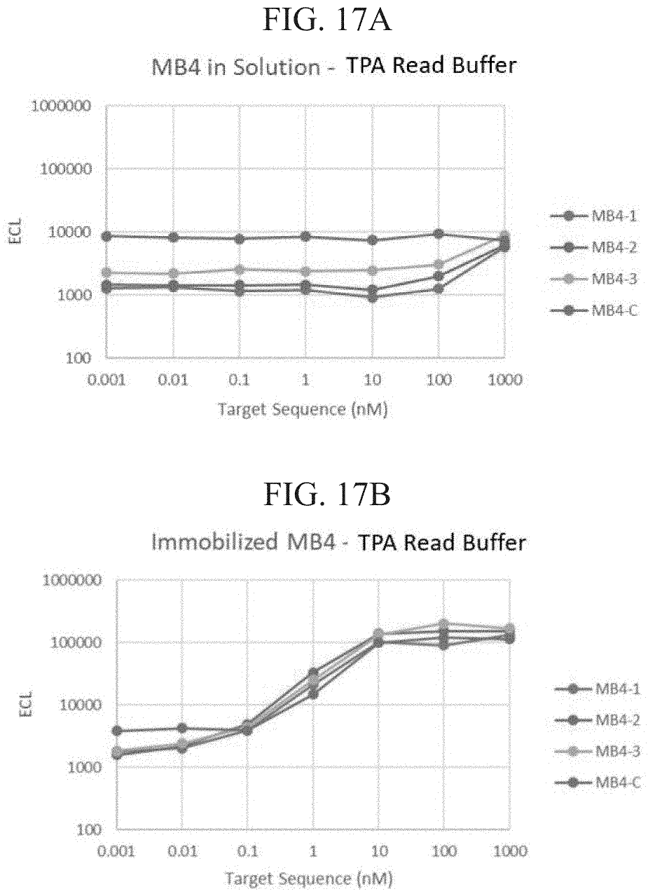

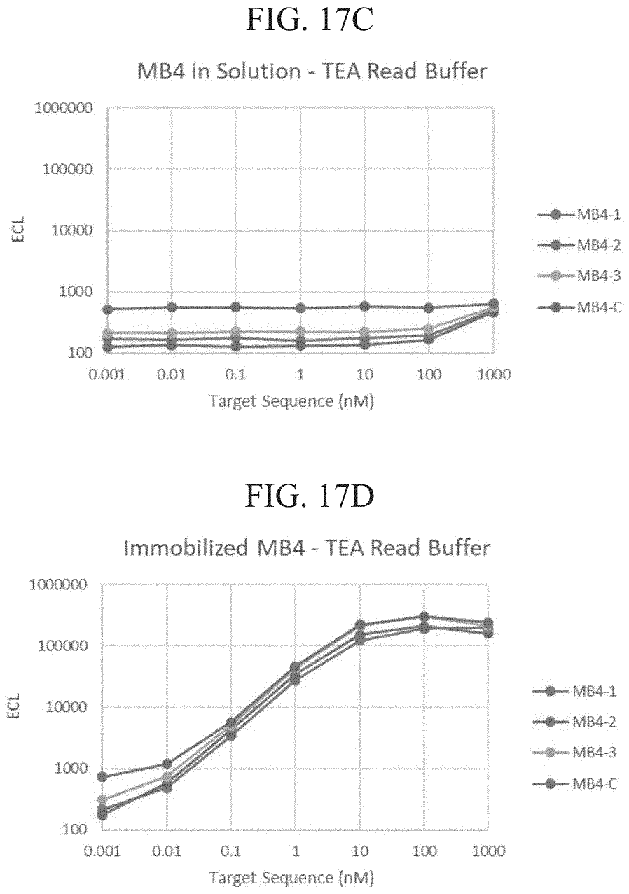

The performance of the MBs were tested in the presence of target oligonucleotide in solution on un-coated small-spot plates, and in the presence of target oligonucleotide immobilized on small-spot streptavidin plates (FIGS. 17A-D). In addition, two different ECL co-reactants were tested. First 50 uL of 0-3000 nM T4.2 biotinylated target oligonucleotide was incubated in the plates (either un-coated or streptavidin coated) at room temperature for 60 min at 705 RPM to immobilize the target oligonucleotide on the streptavidin coated plates. Second 25 uL of 300 nM MBs were added to the plate and incubated at 705 RPM for 30 mM at 55° C. followed by 30 mM at room temperature. Finally, 75 uL of 2×read buffer containing ECL co-reactant TPA (TPA Read Buffer) or 75 uL of 1.2M TEA (TEA Read Buffer) with 850 mM NaCL was added to the wells and the plates were read. The final concentration of T4.2 was 0-1000 nM, the final concentration of MB was 50 nM, and the final concentration of TEA was 600 mM.

The ECL signal from the quenched MBs are lower than that of the control (MB4-C) suggesting efficient quenching (in FIGS. 17A, 17B, 17C, and 17D, MB4-C is the top line at the lowest concentration of target oligonucleotide). BHQ2 and Iowa Black appears to be the most efficient quenchers of the S-TAG ECL label tested. The dequenching on the MBs, in solution or when immobilized, can be observed as the ECL signal recovers to the same level as the control at the highest concentration of target oligonucleotide (FIGS. 17A-D).

Immobilization of the MB to the surface by hybridization to the immobilized target oligonucleotide dramatically increase assay performance and dynamic range (FIGS. 17B and 17D). The dynamic range can be further increased by an order of magnitude in read buffer containing TEA (FIG. 17D) compared to read buffer containing TPA (FIG. 17B). Without being bound by any theory, it is believed that this is a result of decreasing the background form species in solution when using a read buffer with decreased radial lifetime.

### Example 11. Performance of ECL-Labeled Oligonucleotide Molecular-Beacon-Like Probes with TEA Co-Reactant in a Wash-Free Assay

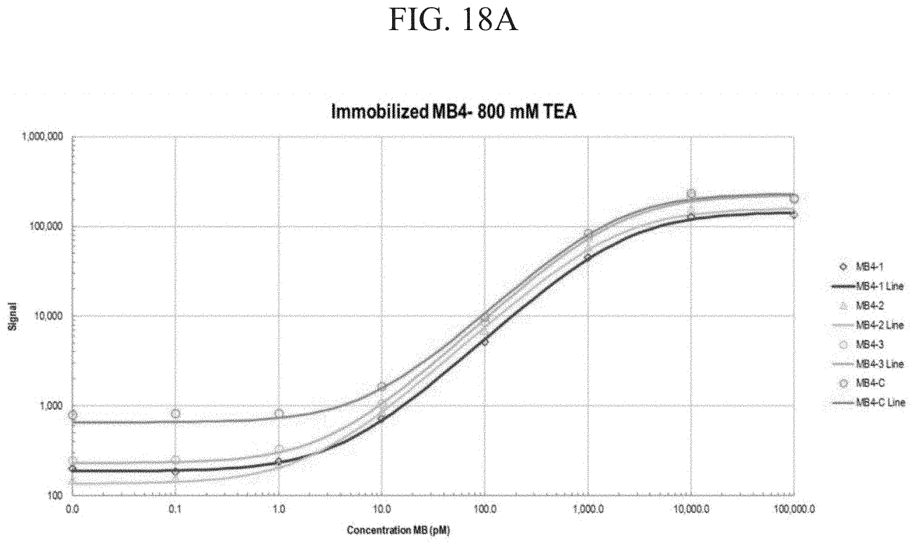

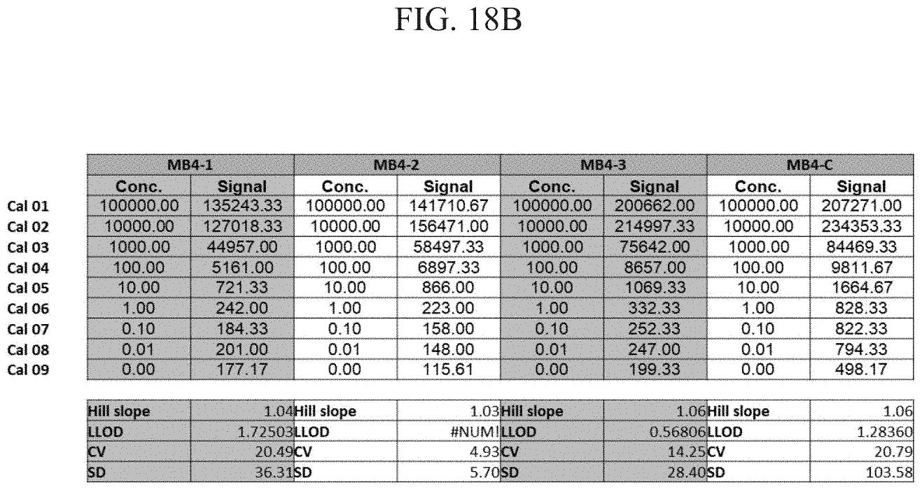

The sensitivity of a wash-free assay over a broad concentration range of target oligonucleotide was tested on small-spot streptavidin plates using the MB probes described in Example 9 (FIGS. 18A-B). Samples were run in triplicates with 18×non-specific binding signal (NSB) for more accurate measurements. First, 40 uL of 0-300 nM T4.2 biotinylated target oligonucleotide was incubated in the streptavidin coated plates at room temperature for 60 min at 705 RPM to immobilize the target oligonucleotide. Second, 10 uL of 750 nM MBs were added to the plate and incubated at 705 RPM for 30 mM at 55° C. followed by 30 mM at room temperature. Finally, 100 uL 1.2M TEA with 850 mM NaCL was added to the wells and the plates were read. The final concentration of T4.2 was 0-100 nM, the final concentration of MB was 50 nM, and the final concentration of TEA was 800 mM. The lower-limit of detection (LLOD) for MB4-2 did not compute due to low CV. The sensitivity of the assay without amplification is around 1 pM. The quenching of the MBs, and decrease in background ECL signal, can be observed compared to the control MB (MB4-C, top line at lowest concentration of target oligonucleotide in FIG. 18A).

### Example 12. Performance of ECL-Labeled Oligonucleotide Molecular-Beacon-Like Probes with TEA Co-Reactant in a Two-Step Wash Free Assay

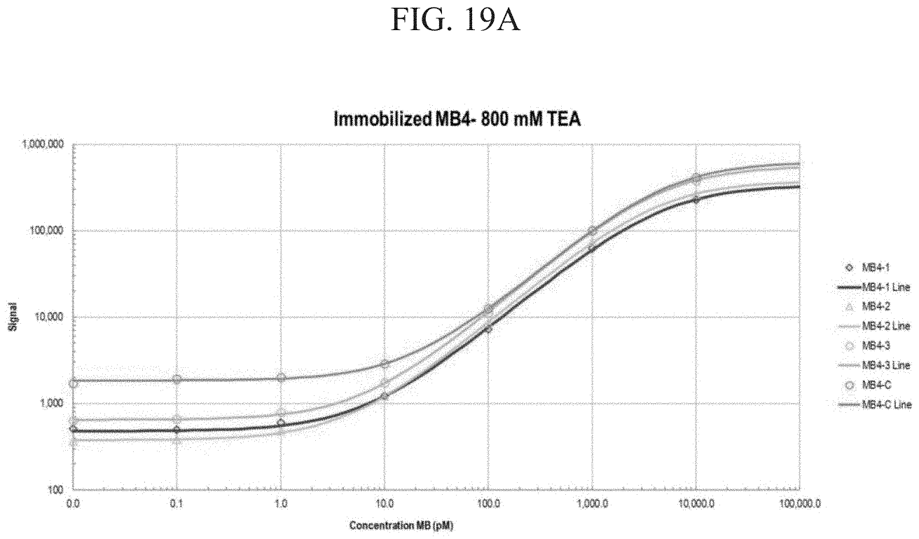

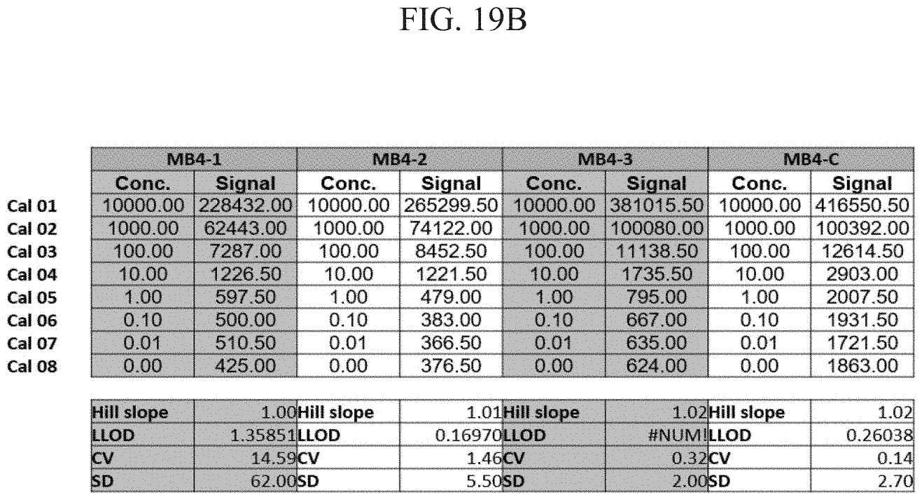

The performance of a 2-step wash free MB assay was tested on a small-spot streptavidin plate. First 40 uL of 0-300 nM T4.2 biotinylated target oligonucleotide was incubated in the the streptavidin coated plates at room temperature for 60 mM at 705 RPM to immobilize the target oligonucleotide. Second a mix of 10 uL of 750 nM MB and 100 uL 1.2M TEA with 850 mM NaCL was added to the wells and incubated at 55° C., 705 RPM for 15 mM followed by 15 mM at room temperature before the plate was read. The final concentration of T4.2 target oligonucleotide was 0-100 nM, the final concentration of MB was 50 nM, and the final concentration of TEA was 800 mM. The results are depicted in FIGS. 19A-B. The background signal increased slightly in the 2-step assay as compared to the assay where the MB was added and incubated prior to addition of the TEA read buffer (Example 11). This could be a result of prolonged incubation of the MBs in the TEA read buffer. In this assay the LLOD for MB4-3 did not compute due to low CV.

The present disclosure is not to be limited in scope by the specific embodiments described herein. Various modifications of the disclosure in addition to those described herein will become apparent to those skilled in the art from the foregoing description and accompanying figures. Such modifications are intended to fall within the scope of the claims. Various publications are cited herein, the disclosures of which are incorporated by reference in their entireties.

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. An electrochemiluminescence (ECL) detection method comprising:
a) providing a substrate comprising an electrode and having a binding reagent immobilized on a surface of the substrate;
b) contacting the substrate with a composition, the composition comprising:
i) a binding partner and/or binding complex comprising an oligonucleotide, wherein the binding reagent binds the binding partner and/or binding complex;
ii) a plurality of ECL-labeled oligonucleotide probes comprising an oligonucleotide sequence that is complementary to an oligonucleotide sequence of the oligonucleotide of the binding partner and/or binding complex; and
iii) an ECL co-reactant that is not TPA;
c) allowing a portion of the plurality of ECL-labeled oligonucleotide probes to hybridize to the oligonucleotide of the binding partner and/or binding complex, wherein the binding partner and/or binding complex is bound by the binding reagent, and wherein another portion of the plurality of ECL-labeled oligonucleotide probes is not hybridized to the oligonucleotide of the binding partner and/or binding complex bound by the binding reagent;
d) selectively dequenching the portion of the plurality of ECL-labeled probes hybridized to the oligonucleotide of the binding partner and/or binding complex;
e) applying a voltage to the electrode to generate ECL; and
f) measuring the ECL wherein the portion of the plurality of ECL-labeled oligonucleotide probes that is not hybridized to the oligonucleotide of the binding partner and/or binding complex is not removed from the composition prior to applying the voltage and measuring the ECL.

2. The method of claim 1, wherein b) contacting the substrate with the composition comprises:
b′) contacting the substrate with a composition comprising the binding partner and/or binding complex;
b″) contacting the substrate with a composition comprising the plurality of ECL-labeled oligonucleotide probes; and
b′″) contacting the substrate with a composition comprising the ECL co-reactant.

3. The method of claim 2, wherein each of steps b′), b″) and b′″) are carried out sequentially.

4. The method of claim 2, wherein at least two of steps b′), b″) and b′″) are carried out simultaneously.

5. The method of claim 1, wherein the method comprises:
b′) contacting the substrate with a first composition comprising the binding partner and/or binding complex, and allowing the binding partner and/or binding complex to immobilize on the surface by binding to the binding reagent; and
b″) contacting the substrate comprising the immobilized binding partner and/or binding complex with a second composition comprising the plurality of ECL-labeled oligonucleotide probes and the ECL co-reactant;
or
b′) contacting the substrate with a first composition comprising the binding partner and/or binding complex and the plurality of ECL-labeled oligonucleotide probes, wherein a portion of the plurality of ECL-labeled oligonucleotide probes are hybridized to the oligonucleotide of the binding partner and/or binding complex, and allowing the binding partner and/or binding complex to immobilize on the surface by binding to the binding reagent; and
b″) contacting the substrate comprising the immobilized binding partner and/or binding complex with a second composition comprising the ECL co-reactant;
or
b′) contacting the substrate with a first composition comprising the binding partner and/or binding complex and allowing the binding partner and/or binding complex to immobilize on the surface by binding to the binding reagent; and
b″) contacting the substrate comprising the immobilized binding partner and/or binding complex with a second composition comprising the plurality of ECL-labeled oligonucleotide probes and allowing a portion of the plurality of ECL-labeled oligonucleotide probes to hybridize to the oligonucleotide of the immobilized binding partner and/or binding complex; and
b′″) contacting the substrate with a third composition comprising the ECL co-reactant.

6. The method claim 1, further comprising washing the substrate following the contacting the substrate with the binding partner and/or binding complex to remove binding partner and/or binding complex not bound by the binding reagent, wherein the washing is prior to contacting the substrate with the composition comprising the plurality of ECL-labeled oligonucleotide probes.

7. An electrochemiluminescence (ECL) detection method comprising:
a) providing a substrate comprising an electrode and having a binding partner and/or binding complex comprising an oligonucleotide immobilized on a surface of the substrate;
b) contacting the substrate with a composition, the composition comprising:
i) a plurality of ECL-labeled oligonucleotide probes comprising an oligonucleotide sequence that is complementary to an oligonucleotide sequence of the oligonucleotide of the binding partner and/or binding complex; and
ii) an ECL co-reactant that is not TPA;
c) allowing a portion of the plurality of ECL-labeled oligonucleotide probes to hybridize to the oligonucleotide of the immobilized binding partner and/or binding complex, and wherein another portion of the plurality of ECL-labeled oligonucleotide probes is not hybridized to the oligonucleotide of the immobilized binding partner and/or binding complex;
d) selectively dequenching the portion of the plurality of ECL-labeled probes hybridized to the oligonucleotide of the binding partner and/or binding complex;
e) applying a voltage to the electrode to generate ECL; and
f) measuring the ECL wherein the portion of the plurality of ECL-labeled oligonucleotide probes that is not hybridized to the oligonucleotide of the binding partner and/or binding complex is not removed from the composition prior to applying the voltage and measuring the ECL.

8. (canceled)

9. (canceled)

10. (canceled)

11. The method of claim 1, wherein the binding partner and/or binding complex comprises an analyte.

12. The method of claim 11, wherein the analyte comprises a peptide or an oligonucleotide.

13. (canceled)

14. The method of claim 1312, wherein the analyte is the oligonucleotide of the binding partner and/or binding complex.

15. (canceled)

16. The method of claim 1411, wherein the analyte is labeled with the oligonucleotide by binding the analyte with a detection reagent comprising the oligonucleotide.

17. The method of claim 1, wherein the oligonucleotide of the binding partner and/or binding complex comprises multiple copies of the sequence complementary to the oligonucleotide sequence of the plurality of the ECL-labeled oligonucleotide probes.

18. The method of claim 17, further comprising, prior to contacting the substrate with the plurality of the ECL-labeled oligonucleotide probes, performing an amplification reaction to generate the multiple copies of the sequence complementary to the oligonucleotide sequence of the plurality of the ECL-labeled oligonucleotide probes.

19. The method of claim 16, wherein the analyte is labeled with the oligonucleotide by binding the analyte with a detection reagent comprising an oligonucleotide primer, and wherein the oligonucleotide primer is extended by a polymerase to generate the oligonucleotide that comprises the multiple copies of the sequence complementary to the oligonucleotide sequence of the ECL-labeled oligonucleotide probes.

20. The method of claim 18, wherein the amplification reaction or primer extension is a rolling circle amplification reaction.

21. The method of claim 1,
wherein the ECL-labeled oligonucleotide probes include a stem-loop or hairpin structure, an ECL label, and a quenching moiety, wherein the quenching moiety is in proximity to the ECL label and quenches the ECL label when the oligonucleotide probe is in a stem-loop or hairpin configuration, but does not quench the ECL label when the stem-loop or hairpin structure is in an open configuration, and
wherein the selectively dequenching comprises hybridizing the portion of the plurality of ECL-labeled oligonucleotide probes to the to the oligonucleotide of the binding partner and/or binding complex in the open configuration.

22. The method of claim 1,
wherein the ECL-labeled oligonucleotide probes comprise an ECL label and a quenching moiety, wherein the quenching moiety is in proximity to the ECL label and quenches the ECL label when the oligonucleotide probe is in a linear confirmation,
wherein the selectively dequenching comprises selectively cleaving the quenching moiety from only the portion of the plurality of ECL-labeled probes hybridized to the oligonucleotide of the binding partner and/or binding complex such that the quenching moiety is released into solution and is no longer in proximity to the ECL label of the hybridized ECL-labeled probe which remains hybridized to the oligonucleotide of the binding partner and/or binding complex after cleavage of the quenching moiety.

23. The method of claim 22, wherein the cleaving is performed by an enzyme selected from the group consisting of a nicking restriction endonuclease, an RNaseH2, and a polymerase having 5′ exonuclease activity.

24. (canceled)

25. (canceled)

26. (canceled)

27. The method of claim 23, wherein the enzyme is a polymerase having 5′ exonuclease activity, and wherein the method further comprises:
hybridizing a primer to the oligonucleotide of the binding partner and/or binding complex at a position 5′ of the hybridized ECL-labeled probe,
allowing the polymerase having 5′ exonuclease activity to extend the primer to the hybridized ECL-labeled probe, wherein the 5′ exonuclease activity cleaves the quenching moiety of the hybridized ECL-labeled probe, and
wherein the ECL-labeled probe comprises a portion that is resistant to the 5′ exonuclease activity.

28. The method of claim 1, wherein the ECL co-reactant is selected from the group consisting of 3-(di-n-propylamino)-propanesulfonic acid; 4-(di-n-propylamino)-butanesulfonic acid; 4-[bis-(2-hydroxyethane)-amino]-butanesulfonic acid; piperidine-N-(3-propanesulfonic acid); azepane-N-(3-propanesulfonic acid); piperidine-N-(3-propionic acid) (PPA); 3-morpholino-2-hydroxypropanesulfonic acid (MOPSO); 3-morpholinepropanesulfonic acid (MOPS); N-(2-hydroxyethyl)piperazine-N′-3-propanesulfonic acid (EPPS); N-(2-hydroxyethyl)piperazine-N′-3-ethanesulfonic acid (BES); piperazine-N,N′-bis(2-ethanesulfonic acid) (PIPES); triethanolamine (TEA); N-2-hydroxypiperazine-N-2-ethanesulfonic acid (HEPES); piperazine-N,N′-bis-4-butanesulfonic acid; homopiperidine-N-3-propanesulfonic acid; piperazine-N,N′-bis-3-propanesulfonic acid; piperidine-N-3-propanesulfonic acid; piperazine-N-2-hydroxyethane-N′-3-methylpropanoate; piperazine-N,N′-bis-3-methylpropanoate; 1,6-diaminohexane-N,N,N′,N′-tetraacetic acid; N,N-bis propyl-N-4-aminobutanesulfonic acid; N-tris(hydroxymethyl)methyl-2-aminoethane sulfonic acid (TES); 1,3-bis[tris(hydroxymethyl)methylamino]propane (bis-Tris propane); 3-dimethylamino-1-propanol; 3-dimethylamino-2-propanol; N,N,N′,N′-tetrapropylpropane-1,3-diamine (TPA dimer); piperazine-N,N′-bis(2-hydroxypropane)sulfonic acid (POPSO) and 2-hydroxy-3-[4-(2-hydroxyethyl)piperazin-1-yl]propane-1-sulfonic acid (HEPPSO), N-butyldiethanolamine (BDEA) 2-dibutylaminoethanol (DBAE), tert-butyldiethanolamine (tBDEA), methyldiethanolamine (MDEA), 3-[Bis-(2-hydroxy-ethyl)-amino]-propane-1-sulfonic acid (DEA-PS), and combinations thereof.

29-34. (canceled)


SEQUENCE LISTING (6 sequences)

SEQ ID NO: 1 - DNA, 29 bp, synthetic construct
  misc_feature 1..29: note = Synthetic Oligonucleotide
  source 1..29: mol_type = other DNA
ccaagcgagccccccatattgtagcttgg

SEQ ID NO: 2 - DNA, 29 bp, synthetic construct
  misc_feature 1..29: note = Synthetic Oligonucleotide
  source 1..29: mol_type = other DNA
ccaagcgagccccccatattgtagcttgg

SEQ ID NO: 3 - DNA, 29 bp, synthetic construct
  misc_feature 1..29: note = Synthetic Oligonucleotide
  source 1..29: mol_type = other DNA
ccaagcgagccccccatattgtagcttgg

SEQ ID NO: 4 - DNA, 29 bp, synthetic construct
  misc_feature 1..29: note = Synthetic Oligonucleotide
  source 1..29: mol_type = other DNA
ccaagcgagccccccatattgtagcttgg

SEQ ID NO: 5 - DNA, 60 bp, synthetic construct
  misc_feature 1..60: note = Synthetic Oligonucleotide
  source 1..60: mol_type = other DNA
aaagatgataagctccggcaagcaatattgtacaatatggggggctccgatataaacaga

SEQ ID NO: 6 - DNA, 60 bp, synthetic construct
  misc_feature 1..60: note = Synthetic Oligonucleotide
  source 1..60: mol_type = other DNA
aaagatgataagctccggcaagcaatattctacaatatggggggctcggatataaacaga